(19) United States
(12) Patent Application Publication
(10) Pub. No.: US 2012/0172339 A1
(43) Pub. Date: Jul. 5, 2012

(54) ANGIOGENIC RESORCINOL DERIVATIVES

(75) Inventors: Alexandros Makriyannis, Watertown, MA (US); Spyridon P. Nikas, Waltham, MA (US); Shariku O. Alapafuja, Willimantic, CT (US)

(73) Assignee: Northeastern University, Boston, MA (US)

(21) Appl. No.: 13/383,036

(22) PCT Filed: Jul. 9, 2010
(86) PCT No.: PCT/US10/41575

§ 371 Date: Mar. 20, 2012

Related U.S. Application Data
(60) Provisional application No. 61/224,695, filed on Jul. 10, 2009.

(51) Int. Cl. A61K 31/05; C07C 69/732; A61K 31/235; C07C 255/36; A61K 31/277; C07C 247/10; A61K 31/655; A61P 9/12; A61P 7/00; A61P 9/00; A61P 27/06; A61P 9/06; A61P 9/04; A61P 17/02; A61P 25/00; A61P 29/00; A61P 35/00; A61P 27/00; C07C 39/17

(52) U.S. Cl. 514/150; 568/743; 514/729; 568/733; 560/59; 514/532; 558/410; 514/520; 552/10

(57) ABSTRACT

Novel resorcinol derivatives and methods of preparation and use are presented. These compounds can stimulate angiogenesis as a biological function triggered by the activation of one cannabinoid receptor distinct from CB1 and CB2. Thus, these compounds are specific ligands for one cannabinoid receptor distinct from CB1 and CB2. The invented compounds, when administered in a therapeutically effective amount to an individual or animal, results in a sufficiently high level of that compound in the individual or animal to cause a physiological response. The physiological response may be useful to treat a number of physiological conditions.

### CROSS REFERENCE TO RELATED APPLICATIONS

This application claims the benefit of U.S. Provisional Application No. 61/224,695, filed Jul. 10, 2009, the entire contents of which are hereby incorporated by reference herein.

### FIELD OF THE INVENTION

The disclosed compounds and methods relate to the field of medicine. More specifically, the disclosed compounds and methods relate to the treatment of cardiovascular disorders.

### BACKGROUND

Presently two G_{i/o} protein coupled cannabinoid receptors, namely CB1 and CB2, have been characterized in mammals and other organisms. The CB1 receptor is very densely distributed through the central nervous system, and at lower levels in various peripheral tissues, including the myocardium, postgangliomic autonomic nerve terminals, and vascular endothelial and smooth muscle cells as well as the liver, skeletal muscle and adipose tissue (Pacher, et al., Pharmacol. Rev. (2006) 58:389-462; Batkai, et al., Circulation (2004) 110:1996-2002; Bonz, et al., J. Cardiovasc. Pharmacol. (2005) 41:657-664; Mukhopadhyay, et al., J. Am. Coll. Cardiol. (2007) 50:528-536; Rajesh, et al., Am. J. Physiol. Heart Circ. Physiol. (2007) 293:H2210-H2218; Rajesh, et al., Br. J. Pharmacol. (2008) 153:347-357; Mallat, et al. Am. J. Physiol. Gastrointest. Liver Physiol. (2008) 294:9-12; Osei-Hyiaman, et al., J. Clin. Invest. (2005) 115:1298-1305; Engeli, et al., Diabetes (2005) 54:2838-2843; Jeong, et al., Cell. Metab. (2008) 7:227-235; Pagotto, et al., Endocr. Rev. (2006) 27:73-100; Cota, et al., J. Clin. Invest. (2003) 112:423-431).

The CB2 receptor is present in immune and hematopoietic cells and recently has also been identified in the brain, myocardium, liver, and human coronary endothelial and smooth muscle cells (Van Sickle, et al., Science (2005) 310:329-332; Gong, et al., Brain Res. (2006) 1071:10-23; Mukhopadhyay,et al., J. Am. Coll. Cardiol. (2007) 50:528-536; Mallat, et al., Am. J. Physiol. Gastrointest. Liver Physiol. (2008) 294:9-12; Rajesh, et al. Am. J. Physiol. Heart Circ. Physiol. (2007) 293:H2210-2218; Rajesh, et al., Br. J. Pharmacol. (2008) 153:347-357).

Some compounds (cannabinergic ligands) can bind to the CB1 and/or CB2 receptors in an individual or animal. In vitro methods for assaying the ability of a compound to bind to CB1 and/or CB2 receptors are known. Results from the in vitro assay correlate with and predict the in vivo ability of that compound to bind to CB1 and/or CB2 receptors and modulate their function(s). When introduced in an individual or animal some of these cannabinergic ligands can bind to and modulate (activate or deactivate) the CB1 and/or CB2 receptors. Examples of some cannabinergic ligands include N-arachidonoyl ethanolamine (anandamide, AEA) and 2-arachidonoylglycerol (2-AG) (both endogenous ligands for the cannabinoid CB1 and CB2 receptors), (−)-Δ⁹-tetrahydrocannabinol (Δ⁹-THC, the principal bioactive constituent of cannabis preparations and exogenous ligand for the cannabinoid CB1 and CB2 receptors) and other synthetic cannabinergic analogs.

Many physiological effects have been associated with modulation of the CB1 and/or CB2 receptors in an individual or animal However, resent findings indicate that some cannabinoid effects are not mediated by either CB1 or CB2 receptors, and in some cases there is compelling evidence to implicate additional receptors in these actions. These include, a transient receptor potential vanilloid 1 (TRPV 1), the orphan G protein-coupled receptor GPR55, atypical cannabinoid receptors in the central nervous system, and an as-yet-unidentified cannabinoid receptor (namely “non-CB1/non-CB2 endothelial cannabinoid receptor” or “non-CB1/non-CB2 anandamide receptor” or “abnormal cannabidiol sensitive receptor”) implicated in the endothelium-dependent vasodilator effect of certain cannabinoids.

Classical exogenous cannabinergic ligands such as Δ⁹-THC as well as the endogenous anandamide and 2-AG are known to have complex cardiovascular effects, a prominent component of which is hypotension (Vollmer, et al., J. Pharm. Pharmacol. (1974) 26:186-198; Varga, et al. FASEB J. (1998) 12:1035-1044; Varga, et al., Eur. J. Pharmacol. (1995) 278:279-283; Stein, et al., Br. J. Pharmacol. (1996) 119:107-114; Varga, et al., Hypertention (1996) 28:682-688; Lake, et al., Hypertention (1997) 29:1204-1210; Calignano, et al., Eur. J. Pharmacol. (1997) 337:R1-R2).

A number of these effects are mediated by the CB1 receptor as shown by the use of the selective CB1 antagonist SR141716 (Varga, et al., Eur. J. Pharmacol. (1995) 278:279-283) or CB1 receptor-deficient mice (Ledent, et al., Science (1999) 283:401-404; Jarai, et al., Proc. Natl. Acad. Sci. U.S.A. (1999) 96:14136-14141).

The cannabinoid compounds abnormal cannabidiol (Abn-CBD, compound 4.2, Table 2) and cannabidiol (CBD, compound 3.2, Table 1) can bind to, and modulate (activate or deactivate), the “non-CB1/non-CB2 endothelial cannabinoid receptor”. More specifically, Abn-CBD (a structural analog of the behaviorally inactive marijuana constituent CBD) is a selective agonist, while CBD is a selective antagonist of the “non-CB1/non-CB2 endothelial cannabinoid receptor”.

Ligands for the “non-CB1/non-CB2 endothelial cannabinoid receptor”, such as Abn-CBD and CBD, can bind to and modulate (activate or deactivate) the “non-CB1/non-CB2 endothelial cannabinoid receptor” and thereby provide a physiological effect in an individual or animal that is useful to treat a condition in that individual or animal. Conditions that may be treated by modulation of the “non-CB1/non-CB2 endothelial cannabinoid receptor” include for example: high blood pressure disease or hypertension; peripheral vascular disease; coronary artery disease; abnormal heart rate; pulmonary hypertension; ocular hypertension or glaucoma; diseases where hypotension is the result of the action of endogenous cannabinoids and drug-induced vasoconstriction is desirable, for example in hypotensive states, such as shock; vasodilatory shock (caused by vascular dilation, as seen for example in cerebral trauma, drug intoxication, heat exposure or septic shock accompanying a gram negative bacterial infection); cardiogenic shock (for example from arrhythmia or heart failure); to achieve selective hemostasis to stop bleeding induced by trauma or surgery; to treat angiogenesis-dependent events involved both in physiological and pathological conditions such as wound healing, placental development, stroke related blockage of blood capillaries, rheumatoid arthritis, diabetic retinopathy and tumor growth.

Classical exogenous cannabinergic ligands such as Δ⁹-THC, in addition to bind at CB1/CB2 cannabinoid receptors, also affect cellular membranes, thereby producing undesirable side effects such as drowsiness, impairment of monoamide oxidase function and impairment of non-receptor mediated brain function. The addictive and psychotropic properties of some cannabinergic ligands also limit their therapeutic value. The cannabinoid compounds Abn-CBD and CBD do not bind to the classical CB1 cannabinoid receptors and they do not have psychotropic activity. Thus, compounds that bind to and modulate the putative “Abn-CBD sensitive receptor” and they do not bind to the CB1 receptor, may provide desirable pharmacological properties without the addictive and psychotropic properties as well as other undesirable properties associated with increased concentrations of classical cannabinoids.

### SUMMARY

Novel resorcinol derivatives represented by the general formulas I, II and III, and methods of preparation and use are presented. These compounds can stimulate angiogenesis as a biological function triggered by the activation of one cannabinoid receptor distinct from CB1 and CB2. Thus, these compounds are specific ligands for one cannabinoid receptor distinct from CB1 and CB2. The invented compounds, when administered in a therapeutically effective amount to an individual or animal, results in a sufficiently high level of that compound in the individual or animal to cause a physiological response. The physiological response may be useful to treat a number of physiological conditions.

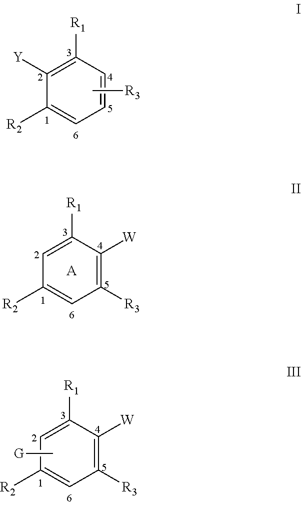

One aspect of the disclosure provides compounds represented by the general formula I and pharmaceutically acceptable (i.e. non-toxic, physiologically acceptable) salts thereof. The general formula I includes all stereoisomers (geometric isomers, diastereomers and enantiomers).

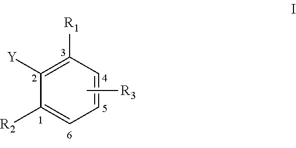

wherein:

R₁ and R₂ are each independently selected from —H, —F, —Cl, —Br, —I, —OH, —SH, —NH₂, —NO₂, —CN, —CF₃, —OC(O)CH₃, —C(O)CH₃, —C(O)CF₃, —C(O)CH═CHCOOH, —O-alkyl, —S-alkyl, —NH-alkyl, —NH(alkyl)₂, —O—P(O)(OR)₂ or —O—P(O)(OH)(OR) (where R is selected from H or alkyl), —P(O)(OR)₂ or —P(O)(OH)(OR) (where R is selected from H or alkyl), —O-alkyl-COOR (where R is selected from H or alkyl), —O-alkyl-NR₄R₅, —O-alkyl-CONR₄R₅, —OC(O)—CH(NH₂)-R₆ (where R₆ is selected from H, CH(OH)CH₃ or alkyl-X₁and X₁ is selected from: H, —NH—C(═NH)NH₂, C(O)NH₂, COOH, SH, SCH₃, OH, NH₂, a substituted or unsubstituted aromatic ring, a substituted or unsubstituted heteroaromatic ring, a substituted or unsubstituted heterocyclic ring).

R₄ and R₅ are each independently selected from H, alkyl, hydroxyalkyl or R₄ and R₅ together comprise part of a 3 to 7 membered saturated heterocyclic ring containing up to one additional heteroatom selected from N, O and S.

R₃ is selected from —H, —F, —Cl, —Br, —I, —OH, —SH, —NH₂, —CN, —N₃, —NCS, —NCO, —SO₂Cl, —SO₂F, —CONH₂, —SO₂NH₂, —COOH, —NO₂, —CHO, —CF₃, —SO₃H, —O—P(O)(OH)₂, —Sn(alkyl)₃, —Si(alkyl)₃, —C≡CH, —CH₂—C≡CH, —CH═CH₂, -fluoroalkyl, -alkyl-R₆, —Z-alkyl-R₆, -alkyl-Z-alkyl-R₆, and R₃ can occupy any position selected from 4, 5 and 6 in formula I.

R₆ is selected from —H, —F, —Cl, —Br, —I, —OH, —SH, —NH₂, —CN, —N₃, —NCS, —NCO, —SO₂Cl, —SO₂F, —CONH₂, —SO₂NH₂, —COOH, —NO₂, —CHO, —CF₃, —SO₃H, —O—P(O)(OH)₂, —Sn(alkyl)₃, —Si(alkyl)₃, C≡CH, —CH═CH₂.

Z is selected from —C≡C—, —CH═CH—, —O—, —S—, —NH—, —C(O)—, —C(O)O—, —OC(O)—, —C(O)NH—, —NHC(O)—, —S(O)—, —SO₂—, —SO₂NH—, —NHSO₂—, —SO₂O— and —OSO₂—.

Y is selected from the following structures:

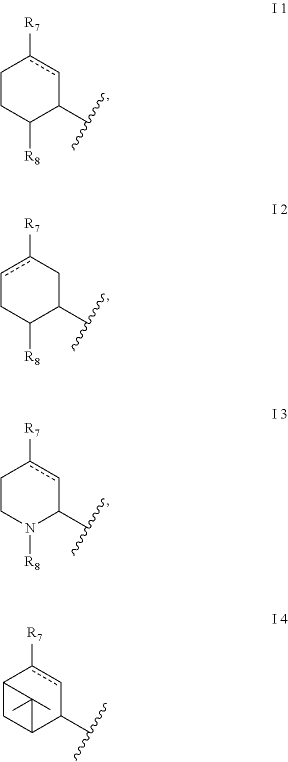

wherein:

The dashed lines independently represent either a single or a double bond.

R₇ is selected from —H, -alkyl, -alkyl-R₉, -alkyl-O-alkyl, -alkyl-O-alkyl-R₉, —C(O)O-alkyl.

R₈ is selected from —H, -alkyl, -alkyl-R₉.

R₉ is selected from —F, —Cl, —Br, —I, —OH, —SH, —NH₂, —NH-alkyl, —N(alkyl)₂, —CN, —N₃, —NCS, —CONH₂, —OC(O)CH₃, —C(O)OCH₃, —SO₂NH₂, —COOH, —NO₂, —CHO, —CF₃, —Sn(alkyl)₃, —Si(alkyl)₃, C≡CH, —CH═CH₂.

The following provisos can apply to some compounds represented by the general formula I.

If Y is I 1 where the dashed line represents a double bond, R₇ is -Me and R₈ is isopropenyl, R₁ is —O—C₁₋₅ alkyl or —O—C₁₋₅alkyl-NR₄R₅, and R₂ is —0—C₁₋₅alkyl or —O—C₁₋₅alkyl-NR₄R₅; then R₃ can not be —H, —F, —Cl, —Br, —I, —C₁₋₃alkyl and —C₁₋₃alkyl-R₆.

If Y is I 1 where the dashed line represents a double bond, R₇ is -Me and R₈ is isopropenyl, R₁ is —OH, and R₂ is —OH; then R₃ can not be —H, and —(CH₂)nCH₃ where n=0-9.

Another aspect of the disclosure provides compounds represented by the general formula II and pharmaceutically acceptable (i.e. non-toxic, physiologically acceptable) salts thereof. The general formula II includes all stereoisomers (geometric isomers, diastereomers and enantiomers).

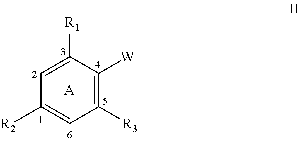

wherein:

R₁, R₂ and R₃ as defined above.

W is selected from the following structures:

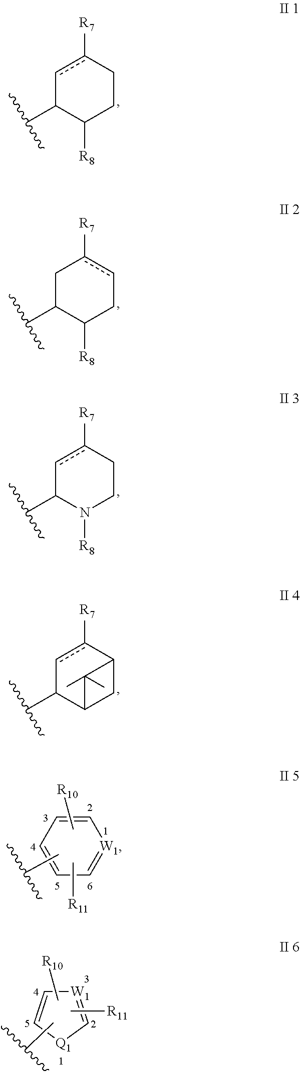

wherein:

The dashed lines independently represent either a single or a double bond.

R₇ and R₈ as defined above.

W₁ is selected from CH and N if W₁ is not bonded to ring A or R₁₀ or R₁₁, or W₁ is C if W₁ is bonded to ring A or R₁₀ or R₁₁. If W₁ is N then it can occupy any position selected from 1, 2, 3, 4, 5 and 6 in II 5, and 2, 3, 4 and 5 in II 6.

Q₁ is selected from CH₂, O, S and NH if Q₁ is not bonded to ring A or R₁₀ or R₁₁, or Q₁ is selected from CH and N if Q₁ is bonded to ring A or R₁₀ or R₁₁.

R₁₀ and R₁₁ are each independently selected from —H, —F, —Cl, —Br, —I, —OH, —OMe, —OEt, —SH, —SMe, -Set, —NH₂, —CN, —N₃, —NCS, —NCO, —SO₂Cl, —SO₂F, —CONH₂, —SO₂NH₂, —COOH, —COOMe, —O—C(O)Me, —NO₂, —CHO, —C(O)CH₃, —C(O)CF₃, —CF₃, —SO₃H, —O—P(O)(OH)₂, —Sn(alkyl)₃, —Si(alkyl)₃, C≡CH, —CH₂—C≡CH, —CH═CH₂.

The following provisos can apply to some compounds represented by the general formula II.

If R₁ and R₂ are both —OH, W is II 1 where the dashed line represents a double bond, R₇ is -Me and R₈ is isopropenyl; then R₃ can not be —H, and —(CH₂)nCH₃ where n=0-9.

If R₁═—OH or —O-alkyl and R₂═—OH, W is II 1 where the dashed line represents a double bond, R₇═—H or -alkyl and R₈ is isopropenyl; then R₃ cannot be —H, —alkyl, —O-alkyl.

If R₁ and R₂ are both —OH, W is 2-isopropenyl-5-methyl-phenyl- or alkyl-substituted 2-isopropenyl-5-methyl-phenyl-; then R₃ can not be —H, and —(CH₂)nCH₃ where n=0-9.

If R₁ and R₂ are both —OH, W is II 1, II 2, II 3 or II 4 where R₇═—H, —C₁₋₅alkyl, —C₁₋₅alkyl—OH, —C₁₋₅alkyl-O—C₁₋₅alkyl, —C₁₋₅alkyl—NH₂, —C₁₋₅alkyl-NH-alkyl, —C₁₋₅alkyl-N(alkyl)₂, and R₈═—H, —C₁₋₅alkyl, —C₁₋₅alkyl—OH, —C₁₋₅alkyl—NH₂, —C₁₋₅alkyl-NH-alkyl, —C₁₋₅alkyl-N(alkyl)₂; then R₃ can not be H, F, Cl, Br, I and —C₁₋₅alkyl.

If R₁ and R₂ are both —OH, W is II 5, where W₁ is CH, R₁₀═—H and R₁₁═—H, —F, —Cl, —Br or I; then R₃ can not be H, F, Cl, Br, I and —C₁₋₅alkyl.

Another aspect of the disclosure provides compounds represented by the general formula III and pharmaceutically acceptable (i.e. non-toxic, physiologically acceptable) salts thereof The general formula III includes all stereoisomers (geometric isomers, diastereomers and enantiomers).

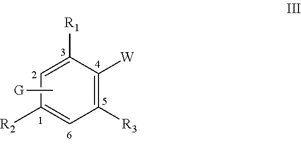

wherein:

R₁, R₂, R₃ and W as defined above.

G can occupy any position selected from 2 and 6 in formula III and G is selected from the following structures:

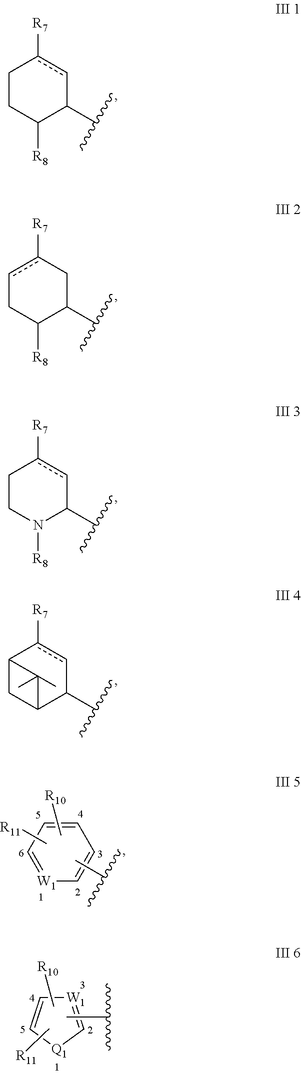

wherein:

The dashed lines independently represent either a single or a double bond.

R₇, R₈, R₁₀, R₁₁, W₁ and Q₁ as defined above.

The following figures are presented for the purpose of illustration only, and are not intended to be limiting.

### BRIEF DESCRIPTION OF THE FIGURES

FIG. 1 is a graphic representation showing ¹H NMR spectrum (500 MHz, CDCl₃, 25° C.) of a mixture of verbenyl etianates 32 and 41 in 2:1 ratio. The expanded scale A shows the C18-methyl protons for the two diastereomers.

FIG. 2 is a graphic representation showing ¹H NMR spectrum (500 MHz, CDCl₃, 25° C.) of verbenyl etianate 32 in which whole picture A and expanded scale B show the C18-methyl protons (δ:0.71). No contamination by the diastereomer41 is observed (δ:0.69 for the C18-methyl protons).

FIG. 3 is a graphic representation showing an expansion of the ¹H NMR spectrum (500 MHz, CDCl₃, 25° C.) of verbenyl etianate 41 showing the C18-methyl protons (δ: 0.69). No contamination by the diastereomer 32 is observed (δ: 0.71 for the C18-methyl protons).

FIG. 4 is a graphic representation showing ¹H NMR spectrum (500 MHz, CDCl₃, 25° C.) of a mixture of enantiomers 7 and 10 (5.7:1 ratio respectively) and Eu(hfc)₃ (Europium tris[3-heptafluoropropylhydroxymethylene)-(+)-camphorate]). Arrows indicate signals due to the —(CH₃)₂— groups and the expanded scale A shows the 3-H protons for the two enantiomers. The ¹H NMR sample was prepared by dissolving ˜8.5 mg of 7, ˜1.5 mg of 10 and ˜31.42 mg of Eu(hfc)₃ in 0.6 ml of CDCl₃.

FIG. 5 is a graphic representation showing expansion of the ¹H NMR spectrum (500 MHz, CDCl₃, 25° C.) of a mixture of 28 and Eu(hfc)₃. Blue arrows indicate presence (˜4%) of the enantiomer 10. The ¹H NMR sample was prepared by dissolving ˜10 mg of 28 and ˜31.42 mg of Eu(hfc)₃ in 0.6 ml of CDCl₃.

FIG. 6 is a graphic representation showing expansion of the ¹H NMR spectrum (500 MHz, CDCl₃, 25° C.) of a mixture of 7 and Eu(hfc)₃. No contamination by the enantiomer 10 is observed. The ¹H NMR sample was prepared by dissolving ˜10 mg of 7 and ˜31.42 mg of Eu(hfc)₃ in 0.6 ml of CDCl₃.

FIG. 7 is a graphic representation showing MMP activity of compounds 5.3, 3.6, 3.15 and (R)-Methanandamide in CB1-KO-HUVEC cells.

FIG. 8 is a graphic representation showing MMP activity of compounds 4.11, 8.2, 3.8 and (R)-Methanandamide in CB1-KO-HUVEC cells.

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein, including GenBank database sequences, are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### A. Definitions

Unless otherwise specifically defined, “acyl” refers to the general formula —C(O)alkyl.

Unless otherwise specifically defined, “alcohol” refers to the general formula alkyl-OH and includes primary, secondary and tertiary variations.

Unless otherwise specifically defined, “alkyl” refers to a linear or branched hydrocarbon radical which may be fully saturated, mono- or polyunsaturated and can include divalent radicals, having from 1 to about 15 carbon atoms. Examples for saturated hydrocarbon radicals include, but are not limited to, groups such as methyl (Me), ethyl (Et), n-propyl, isopropyl, n-butyl, t-butyl, isobutyl, sec-butyl, homologs and isomers of, for example, n-pentyl, n-hexyl, n-heptyl, n-octyl, 1,1-dimethyl-heptyl, 1,2-dimethyl-heptyl, and the like. An unsaturated alkyl group includes one or more double bonds, triple bonds or combinations thereof. Examples of unsaturated alkyl groups include but are not limited to, vinyl, propenyl, isopropenyl, crotyl, 2-isopentenyl, allenyl, butenyl, butadienyl, pentenyl, pentadienyl, 3-(1,4-pentadienyl), hexenyl, hexadienyl, ethynyl, propynyl, butynyl, and higher homologs and isomers. The term “divalent alkyl radicals” unless otherwise specifically defined refers to the general formula: -alkyl-. The term “C₁₋ₘ-alkyl” refers to an alkyl having from 1 to about m carbon atoms.

Unless otherwise specifically defined, “alkoxy” refers to the general formula —O-alkyl.

Unless otherwise specifically defined, “alkylamino” refers to the general formula —(NH)-alkyl.

Unless otherwise specifically defined, “di-alkylamino” refers to the general formula —N-(alkyl)₂. Unless otherwise specifically limited di-alkylamino includes cyclic amine compounds such as piperidine and morpholine.

Unless otherwise specifically defined, “aroyl” refers to the general formula —C(O)-aryl.

Unless otherwise specifically defined, “aryl” or “aromatic ring” refers to a polyunsaturated, aromatic hydrocarbon, which can be a single ring or multiple rings (from 1 to 3 rings) which are fused together or linked covalently and can include “divalent radicals”. The term “divalent aryl radicals” unless otherwise specifically defined refers to the general formula: -aryl-. Examples of aryl groups include but are not limited to, phenyl, biphenyl, and naphthyl.

Unless otherwise specifically defined, “cycloalkyl” or “cycloalkyl ring” refers to a saturated or partially saturated ring structure having about 3 to about 8 ring members that has only carbon atoms as ring atoms and can include divalent radicals. The term “divalent cycloalkyl radicals” unless otherwise specifically defined refers to the general formula: -cycloalkyl-. Examples of cycloalkyl groups include but are not limited to, cyclopropyl, cyclobutyl, cyclopentyl, cyclohexyl, cyclohexene.

Unless otherwise specifically defined, “halogen” refers to an atom selected from fluorine, chlorine, bromine and iodine.

Unless otherwise specifically defined, “heterocyclic” or “heterocyclic ring” refers to a saturated ring structure having about 3 to about 8 ring members that has carbon atoms and one or more heteroatoms, including oxygen, nitrogen and/or sulfur, as ring atoms. The term “heterocyclic” or “heterocyclic ring” can include “divalent radicals”. The term “divalent heterocyclic radicals” unless otherwise specifically defined refers to the general formula: -heterocyclic-. Examples of heterocyclic groups include but are not limited to, oxetane, thietane, azetidine, diazetidine, tetrahydrofuran, thiolane, pyrrolidine, dioxolane, oxathiolane, imidazolidine, dioxane, piperidine, morpholine, piperazine, and their derivatives.

Unless otherwise specifically defined, “heteroaryl” or “heteroaromatic ring” refers to aryl groups (or rings) that contain one or more heteroatoms selected from oxygen, nitrogen and/or sulfur as ring atoms. Heteroaryl groups (or rings) also include fused polycyclic systems in which one or more monocyclic aryl or monocyclic heteroaryl group is fused to another heteroaryl group. “Heteroaryl” can include “divalent radicals”, the term “divalent heteroaryl radicals” unless otherwise specifically defined refers to the general formula: -heteroaryl-. Examples of heteroaryl groups include but are not limited to, furanyl, thienyl, pyrrolyl, oxazolyl, thiazolyl, isoxazolyl, pyrazolyl, imidazolyl, oxadiazolyl, pyridinyl, pyrimidinyl, purinyl, benzoxazolyl, benzothiazolyl, benzimibazolyl, benzofuranyl, indolyl, quinolinyl, quinoxalinyl.

Unless otherwise specifically limited the term substituted means substituted by a below-described substituent group in any possible position. Substituent groups for the above moieties useful in this disclosure are those groups that do not significantly diminish the biological activity of the disclosed compound. Substituent groups that do not significantly diminish the biological activity of the disclosed compound include, for example, H, halogen, N₃, NCS, CN, NO₂, NX₁X₂, OX₃, C(X₃)₃, OAc, O-acyl, O-aroyl, NH-acyl, NH-aroyl, NHCOalkyl, CHO, C(halogen)₃, COOX₃, SO₃H, PO₃H₂, SO₂NX₁X₂, CONX₁X₂, alkyl, alcohol, alkoxy, alkylmercapto, alkylamino, di-alkylamino, sulfonamide, thioalkoxy or methylene dioxy when the substituted structure has two adjacent carbon atoms, wherein X₁ and X₂ each independently comprise H or alkyl, or X₁ and X₂ together comprise part of a heterocyclic ring having about 4 to about 7 ring members and optionally one additional heteroatom selected from O, N or S, or X₁ and X₂ together comprise part of an imide ring having about 5 to about 6 members and X₃ comprises H, alkyl, hydroxyloweralkyl, or alkyl-NX₁X₂. Unless otherwise specifically limited a substituent group may be in any possible position.

The disclosed compounds may be isolated from a naturally occurring or synthetic material. The isolated compound may be contemporaneously or subsequently “purified” or “substantially purified”. As used herein a “purified” or “substantially purified” compound means a compound that has been processed to a desired purity. A person of ordinary skill can establish the desired purity for a use and method to achieve that purity without undue effort. The purified compound may be used in any disclosed embodiment.

As used herein a “therapeutically effective amount” of a compound, is the quantity of a compound which, when administered to an individual or animal, results in a discernible physiological effect in the individual or animal. The compounds disclosed herein, and pharmaceutically acceptable salts thereof, have pharmacological properties when administered in therapeutically effective amounts for providing a physiological effect useful to treat a number of physiological conditions. Typically, a “therapeutically effective amount” of a compound is believed to range from about 5 mg/day to about 1,000 mg/day. As used herein, an “individual” refers to a human. An “animal” refers to, for example, veterinary animals, such as dogs, cats, horses and the like, and farm animals, such as cows, pigs and the like.

### B. Embodiments

The present disclosure relates generally to chemical compounds with angiogenic activity. These chemical compounds are specific ligands for one cannabinoid receptor distinct from CB1 and CB2. The disclosure is more particularly concerned with new resorcinol derivatives that activate angiogenic responses in endothelial cells by acting on non CB1 and non CB2 receptor subgroups, and use of these derivatives to treat pathological conditions arising from blood pressure, vasoconstriction, heart rate, and angiogenesis dependent events.

The present disclosure provides compounds (for examples see Tables 1, 2, and 3). The compounds activate angiogenic responses. In some embodiments the compounds can be new and structurally improved ligands for an as-yet-unidentified cannabinoid receptor distinct from CB1 and CB2. In some embodiments the compounds comprise bis-substituted resorcinol derivatives as novel ligands for an as-yet-unidentified cannabinoid receptor distinct from CB1 and CB2.

In one aspect, the disclosure provides compounds represented by the general formula I and pharmaceutically acceptable (i.e. non-toxic, physiologically acceptable) salts thereof. The general formula I includes all stereoisomers (geometric isomers, diastereomers and enantiomers).

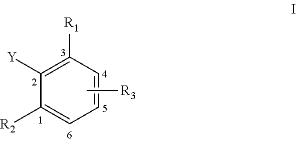

wherein:

R₁ and R₂ are each independently selected from —H, —F, —Cl, —Br, —I, —OH, —SH, —NH₂, —NO₂, —CN, —CF₃, —OC(O)CH₃, —C(O)CH₃, —C(O)CF₃, —C(O)CH═CHCOOH, —O-alkyl, —S-alkyl, —NH-alkyl, —NH(alkyl)₂, —O—P(O)(OR)₂ or —O—P(O)(OH)(OR) (where R is selected from H or alkyl), —P(O)(OR)₂ or —P(O)(OH)(OR) (where R is selected from H or alkyl), —O-alkyl-COOR (where R is selected from H or alkyl), —O-alkyl-NR₄R₅, —O-alkyl-CONR₄R₅, —OC(O)—CH(NH₂)—R₆ (where R₆ is selected from H, CH(OH)CH₃ or alkyl-Xᵢ and X₁ is selected from: H, —NH—C(═NH)NH₂, C(O)NH₂, COOH, SH, SCH₃, OH, NH₂, a substituted or unsubstituted aromatic ring, a substituted or unsubstituted heteroaromatic ring, a substituted or unsubstituted heterocyclic ring).

R₄ and R₅ are each independently selected from H, alkyl, hydroxyalkyl or R₄ and R₅ together comprise part of a 3 to 7 membered saturated heterocyclic ring containing up to one additional heteroatom selected from N, O and S.

R₃ is selected from —H, —F, —Cl, —Br, —I, —OH, —SH, —NH₂, —CN, —N₃, —NCS, —NCO, —SO₂Cl, —SO₂F, —CONH₂, —SO₂NH₂, —COOH, —NO₂, —CHO, —CF₃, —SO₃H, —O—P(O)(OH)₂, —Sn(alkyl)₃, —Si(alkyl)₃, —C≡CH, —CH₂—C≡CH, —CH═CH₂, -fluoroalkyl, -alkyl-R₆, —Z-alkyl-R₆, -alkyl-Z-alkyl-R₆, and R₃ can occupy any position selected from 4, 5 and 6 in formula I.

R₆ is selected from —H, —F, —Cl, —Br, —I, —OH, —SH, —NH₂, —CN, —N₃, —NCS, —NCO, —SO₂Cl, —SO₂F, —CONH₂, —SO₂NH₂, —COOH, —NO₂, —CHO, —CF₃, —SO₃H, —O—P(O)(OH)₂, —Sn(alkyl)₃, —Si(alkyl)₃, —C≡CH, —CH═CH₂.

Z is selected from —C≡C—, —CH═CH—, —O—, —S—, —NH—, —C(O)—, —C(O)O—, —OC(O)—, —C(O)NH—, —NHC(O)—, —S(O)—, —SO₂—, —SO₂NH—, —NHSO₂—, —SO₂O— and —OSO₂—.

Y is selected from the following structures:

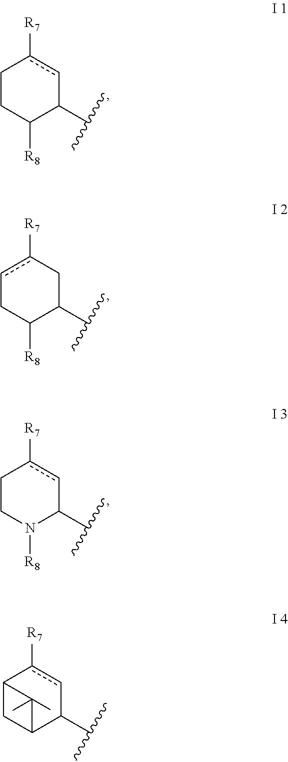

wherein:

The dashed lines independently represent either a single or a double bond.

R₇ is selected from —H, -alkyl, -alkyl-R₉, -alkyl-O-alkyl, -alkyl-O-alkyl-R₉, —C(O)O-alkyl.

R₈ is selected from —H, -alkyl, -alkyl-R₉.

R₉ is selected from —F, —Cl, —Br, —I, —OH, —SH, —NH₂, —NH-alkyl, —N(alkyl)₂, —CN, —N₃, —NCS, —CONH₂, —OC(O)CH₃, —C(O)OCH₃, —SO₂NH₂, —COOH, —NO₂, —CHO, —CF₃, —Sn(alkyl)₃, —Si(alkyl)₃, —C≡CH, —CH═CH₂.

The following provisos can apply to some compounds represented by the general formula I.

If Y is I 1 where the dashed line represents a double bond, R₇ is -Me and R₈ is isopropenyl, R₁ is —O—C₁₋₅ alkyl or —O—C₁₋₅ alkyl-NR₄R₅, and R₂ is ^{—O—C}₁₋₅ alkyl or —O—C₁₋₅alkyl-NR₄R₅; then R₃ can not be —H, —F, —Cl, —Br, —I, —C₁₋₃alkyl and —C₁₋₃alkyl-R₆.

If Y is I 1 where the dashed line represents a double bond, R₇ is -Me and R₈ is isopropenyl, R₁ is —OH, and R₂ is —OH; then R₃ can not be —H, and —(CH₂)nCH₃ where n=0-9.

In some embodiments of the compound of Formula I, R₁ and R₂ are each independently selected from —OH and —SH. Furthermore, R₁ and R₂ are each independently selected from —O-alkyl and -S-alkyl in other embodiments. In additional embodiments, R₃ is selected from —H. In certain exemplary embodiments, R₃ is selected from —F, —Cl, —Br, and —I. R₃ can also be —OH, —SH, and —NH₂ or alternatively R₃ is selected from —CN, —N₃, —NCS, and —NCO. R₃ can also be anyone of —SO₂Cl, —SO₂F, —CONH₂, —SO₂NH₂, and —COOH.

In particular cases, R₃ is selected from —NO₂, —CHO, —CF₃, and —SO₃H. In alternative embodiments, R₃ is selected from —C≡CH, —CH₂—C≡CH, and —CH═CH₂. Furthermore, R₃ can be selected from -alkyl-R₆.

In some embodiments, R₃ is at position 5 in formula I.

In certain embodiments, R₆ is selected from —H, —F, —Cl, —Br, and —I. In other embodiments, R₆ is selected from —OH, —SH, —NH₂, —CN, —N₃, —NCS, and —NCO. In still other exemplary embodiments, R₆ is selected from —SO₂Cl, —SO₂F, —CONH₂, —SO₂NH₂, —COOH, —NO₂, —CHO, —CF₃, and —SO₃H. In more embodiments, R7 is selected from -alkyl. In further embodiments, R7 is selected from -alkyl-R9. In certain examples, R₉ is selected from —F, —Cl, —Br, and —I. In other instances, R₉ is selected from —OH, —SH, —NH₂, and —NH-alkyl. In some cases, R₉ is selected from —N(alkyl)₂, —CN, —N₃, —NCS, and —CONH₂.

R₉ can also be selected from —OC(O)CH₃, —C(O)OCH₃, —SO₂NH₂, —COOH, —NO₂, —CHO, and —CF₃. In some embodiments, R8 is selected from -alkyl.

In another aspect, compounds represented by the general formula II and pharmaceutically acceptable (i.e. non-toxic, physiologically acceptable) salts thereof are provided. The general formula II includes all stereoisomers (geometric isomers, diastereomers and enantiomers).

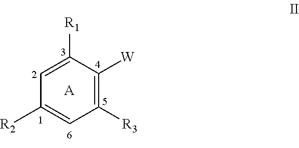

wherein:

R₁, R₂ and R₃ as defined above.

W is selected from the following structures:

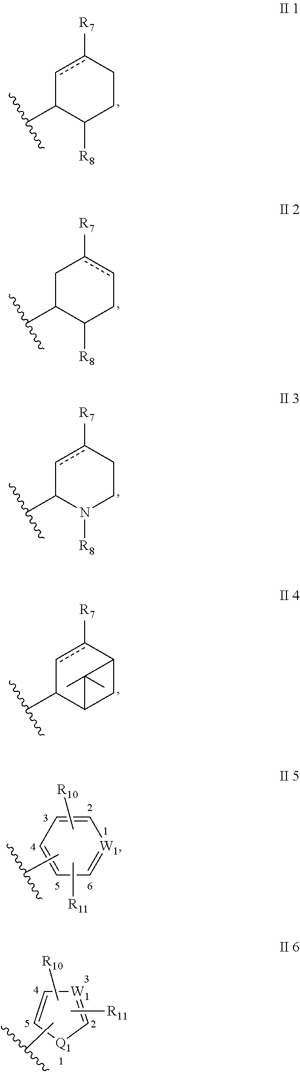

wherein:

The dashed lines independently represent either a single or a double bond.

R₇ is selected from —H, -alkyl, -alkyl-R₉, -alkyl-O-alkyl, -alkyl-O-alkyl-R₉, —C(O)O-alkyl.

R₈ is selected from —H, -alkyl, -alkyl-R₉.

W₁ is selected from CH and N if W₁ is not bonded to ring A or R₁₀ or R₁₁, or W₁ is C if W₁ is bonded to ring A or R₁₀ or R₁₁. If W₁ is N then it can occupy any position selected from 1, 2, 3, 4, 5 and 6 in II 5, and 2, 3, 4 and 5 in II 6.

Q₁ is selected from CH₂, O, S and NH if Q₁ is not bonded to ring A or R₁₀ or R₁₁, or Q₁ is selected from CH and N if Q₁ is bonded to ring A or R₁₀ or R₁₁.

R₁₀ and R₁₁ are each independently selected from —H, —F, —Cl, —Br, —I, —OH, —OMe, —OEt, —SH, —SMe, -Set, —NH₂, —CN, —N₃, —NCS, —NCO, —SO₂Cl, —SO₂F, —CONH₂, —SO₂NH₂, —COOH, —COOMe, —O—C(O)Me, —NO₂, —CHO, —C(O)CH₃, —C(O)CF₃, —CF₃, —SO₃H, —O—P(O)(OH)₂, —Sn(alkyl)₃, —Si(alkyl)₃, —C≡CH, —CH₂—C≡CH, —CH═CH₂.

The following provisos can apply to some compounds represented by the general formula II.

If R₁ and R₂ are both —OH, W is II 1 where the dashed line represents a double bond, R₇ is -Me and R₈ is isopropenyl,; then R₃ can not be —H, and —(CH₂)nCH₃ where n=0-9.

If R₁═—OH or —O-alkyl and R₂═—OH, W is II 1 where the dashed line represents a double bond, R₇═—H or -alkyl and R₈ is isopropenyl; then R₃ cannot be —H, —alkyl, —O-alkyl.

If R₁ and R₂ are both —OH, W is 2-isopropenyl-5-methyl-phenyl- or alkyl-substituted 2-isopropenyl-5-methyl-phenyl-; then R₃ can not be —H, and —(CH₂)nCH₃ where n=0-9.

If R₁ and R₂ are both —OH, W is II 1, II 2, II 3 or II 4 where R₇═—H, —C₁₋₅alkyl, —C₁₋₅alkyl—OH, —C₁₋₅alkyl-O—C₁₋₅alkyl, —C₁₋₅alkyl—NH₂, —C₁₋₅alkyl-NH-alkyl, —C₁₋₅alkyl-N(alkyl)₂, and R₈═—H, —C₁₋₅alkyl, —C₁₋₅alkyl—OH, —C₁₋₅alkyl—NH₂, —C₁₋₅alkyl-NH-alkyl, —C₁₋₅alkyl-N(alkyl)₂; then R₃ can not be H, F, Cl, Br, I and —C₁₋₅alkyl.

If R₁ and R₂ are both —OH, W is II 5, where W₁ is CH, R₁₀═—H and R₁₁═—H, —F, —Cl, —Br or I; then R₃ can not be H, F, Cl, Br, I and —C₁₋₅alkyl.

In some embodiments of this aspect, R₁ and R₂ are each independently selected from —OH and —SH. Furthermore, R₁ and R₂ are each independently selected from —O-alkyl and —S-alkyl in other embodiments. In additional embodiments, R₃ is selected from —H. In certain exemplary embodiments, R₃ is selected from —F, —Cl, —Br, and —I. R₃ can also be —OH, —SH, and —NH₂ or alternatively R₃ is selected from —CN, —N₃, —NCS, and —NCO. R₃ can also be anyone of —SO₂Cl, —SO₂F, —CONH₂, —SO₂NH₂, and —COOH.

In particular cases, R₃ is selected from —NO₂, —CHO, —CF₃, and —SO₃H. In alternative embodiments, R₃ is selected from —C≡CH, —CH₂—C≡CH, and —CH═CH₂. Furthermore, R₃ can be selected from -alkyl-R₆.

In some embodiments, R₃ is at position 5 in formula II.

In certain embodiments, R₆ is selected from —H, —F, —Cl, —Br, and —I. In other embodiments, R₆ is selected from —OH, —SH, —NH₂, —CN, —N₃, —NCS, and —NCO. In still other exemplary embodiments, R₆ is selected from —SO₂Cl, —SO₂F, —CONH₂, —SO₂NH₂, —COOH, —NO₂, —CHO, —CF₃, and —SO₃H. In more embodiments, R7 is selected from -alkyl. In further embodiments, R7 is selected from -alkyl-R9. In certain examples, R₉ is selected from —F, —Cl, —Br, and —I. In other instances, R₉ is selected from —OH, —SH, —NH₂, and —NH-alkyl. In some cases, R₉ is selected from —N(alkyl)₂, —CN, —N₃, —NCS, and —CONH₂.

R₉ can also be selected from —OC(O)CH₃, —C(O)OCH₃, —SO₂NH₂, —COOH, —NO₂, —CHO, and —CF₃. In some embodiments, R8 is selected from -alkyl.

Another aspect of the disclosure provides compounds represented by the general formula III and pharmaceutically acceptable (i.e. non-toxic, physiologically acceptable) salts thereof. The general formula III includes all stereoisomers (geometric isomers, diastereomers and enantiomers).

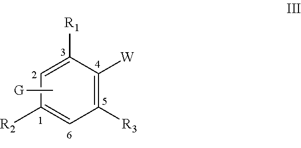

wherein:

R₁, R₂, R₃ and W as defined above.

G can occupy any position selected from 2 and 6 in formula III and G is selected from the following structures:

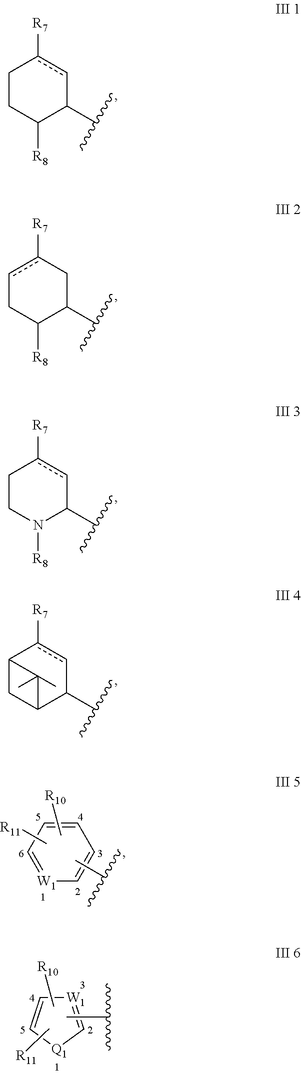

wherein:

The dashed lines independently represent either a single or a double bond.

R₇ is selected from —H, -alkyl, -alkyl-R₉, -alkyl-O-alkyl, -alkyl-O-alkyl-R₉, —C(O)O-alkyl.

R₈ is selected from —H, -alkyl, -alkyl-R₉.

W₁ is selected from CH and N if W₁ is not bonded to ring A or R₁₀ or R₁₁, or W₁ is C if W₁ is bonded to ring A or R₁₀ or R₁₁. If W₁₁is N then it can occupy any position selected from 1, 2, 3, 4, 5 and 6 in II 5, and 2, 3, 4 and 5 in II 6.

Q₁ is selected from CH₂, O, S and NH if Q₁ is not bonded to ring A or R₁₀ or R₁₁, or Q₁ is selected from CH and N if Q₁ is bonded to ring A or R₁₀ or R₁₁.

In some embodiments of this aspect, R₁ and R₂ are each independently selected from —OH and —SH. Furthermore, R₁ and R₂ are each independently selected from —O-alkyl and —S-alkyl in other embodiments. In additional embodiments, R₃ is selected from —H. In certain exemplary embodiments, R₃ is selected from —F, —Cl, —Br, and —I. R₃ can also be —OH, —SH, and —NH₂ or alternatively R₃ is selected from —CN, —N₃, —NCS, and —NCO. R₃ can also be anyone of —SO₂Cl, —SO₂F, —CONH₂, —SO₂NH₂, and —COOH.

In particular cases, R₃ is selected from —NO₂, —CHO, —CF₃, and —SO₃H. In alternative embodiments, R₃ is selected from —C≡CH, —CH₂—C≡CH, and —CH═CH₂. Furthermore, R₃ can be selected from -alkyl-R₆.

In some embodiments, R₃ is at position 5 in formula III.

In certain embodiments, R₆ is selected from —H, —F, —Cl, —Br, and —I. In other embodiments, R₆ is selected from —OH, —SH, —NH₂, —CN, —N₃, —NCS, and —NCO. In still other exemplary embodiments, R₆ is selected from —SO₂Cl, —SO₂F, —CONH₂, —SO₂NH₂, —COOH, —NO₂, —CHO, —CF₃, and —SO₃H. In more embodiments, R7 is selected from -alkyl. In further embodiments, R7 is selected from -alkyl-R9. In certain examples, R₉ is selected from —F, —Cl, —Br, and —I. In other instances, R₉ is selected from —OH, —SH, —NH₂, and —NH-alkyl. In some cases, R₉ is selected from —N(alkyl)₂, —CN, —N₃, —NCS, and —CONH₂.

R₉ can also be selected from —OC(O)CH₃, —C(O)OCH₃, —SO₂NH₂, —COOH, —NO₂, —CHO, and —CF₃. In some embodiments, R8 is selected from -alkyl.

For aspects disclosed herein, promotion of angiogenic responses under in vitro and ex vivo conditions as well as increasing in MMP (matrix metalloproteinase) activity, correlates well with the ability of anandamide and its synthetic analog (R)-methanandamide to bind to and modulate the putative “non CB1/non CB2 anandamide receptor”. Thus, the compounds described herein where tested using in vitro angiogenesis assays (cord formation by endothelial cells in extracellular matrix mimicking matrigel), ex vivo angiogenesis assay (sprouting or blood capillary formation under ex vivo conditions) as well as for MMP activity in CB1 receptor knock out human umbilical vein endothelial cells (HUVEC) (CB1-KO-HUVEC).

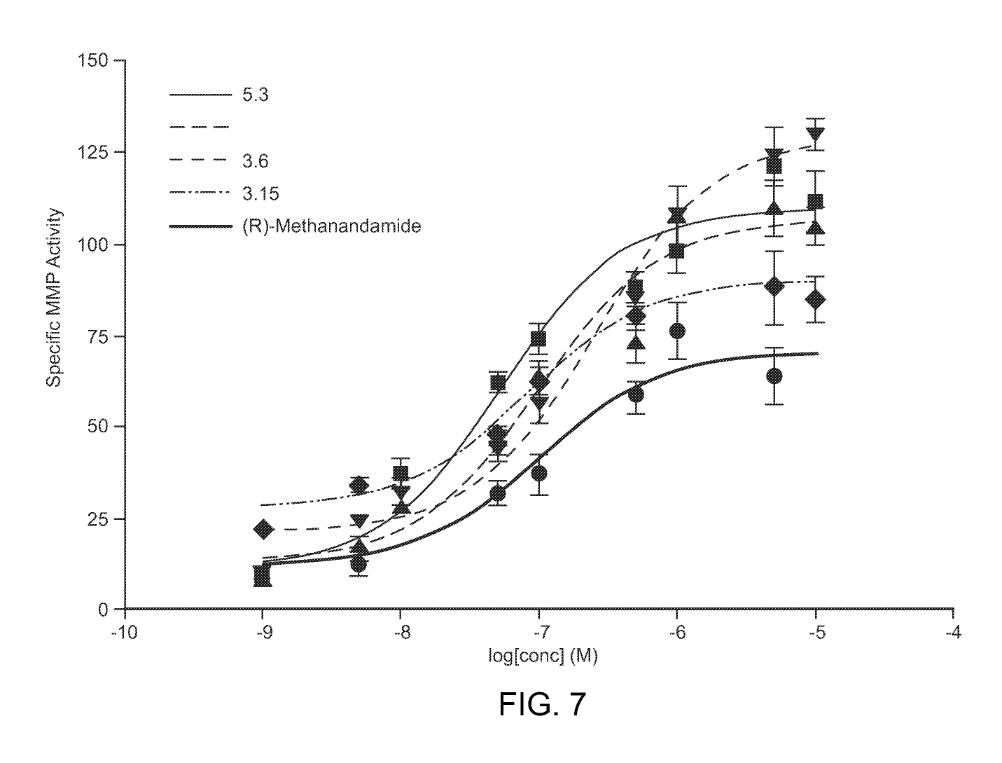

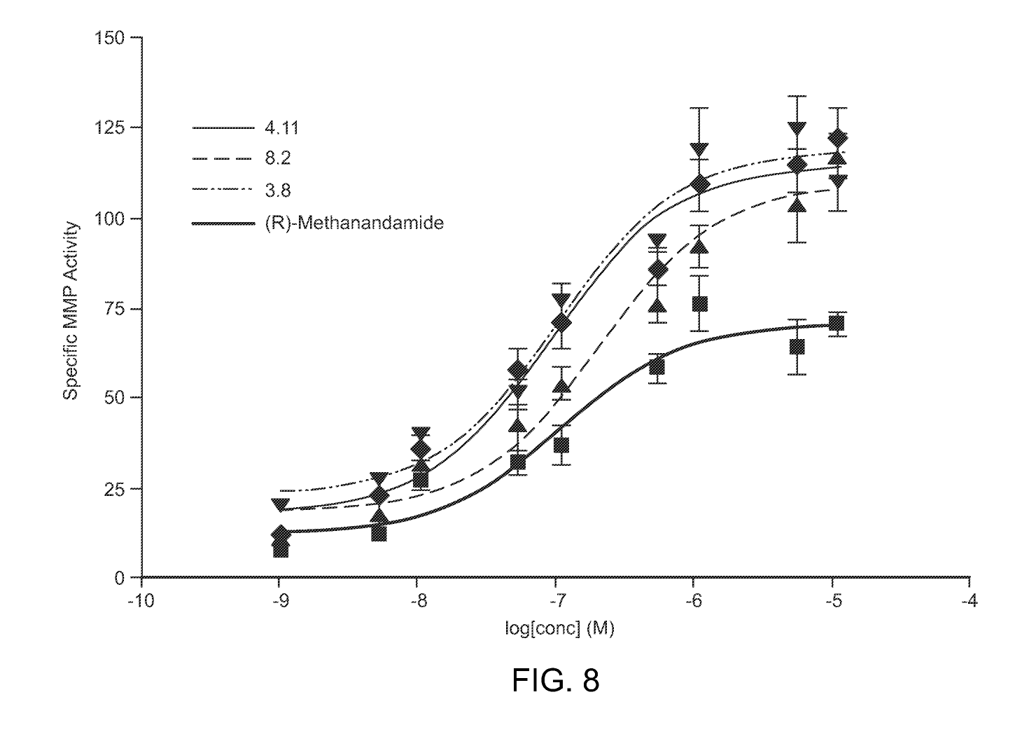

Biological test results of some synthesized compounds shown enhanced ability to bind to the putative “non CB1/non CB2 anandamide receptor” when compared to Abn-CBD, CBD and (R)-methanandamide. This can be seen by comparison of the “angiogenic score” (Table 4) of compounds 4.8, 5.3, 3.15 for example, with Abn-CBD (4.2) and CBD (3.2); and by comparison of the “specific MMP activity” (FIGS. 7 and 8) of compounds 5.3, 3.6, 3.15, 4.11, 8.2 and 3.8 with (R)-methanandamide. Biological test results of some synthesized compounds shown reduced ability to bind to the central (CB1) and the peripheral (CB2) cannabinoid receptors. This can be seen in the binding affinity (Ki) values given in Table 5.

This was a surprising discovery that allowed the invertors to obtain novel compounds with angiogenic activity that are ligands for the putative “non CB1/non CB2 anandamide receptor” without having addictive and psychotropic properties. It is believed that these compounds are substantially pure agonists and antagonists of an as-yet-unidentified cannabinoid receptor distinct from CB1 and CB2.

The disclosed compounds in any formula, embodiment or variation include any and all possible isomers and stereoisomers. The content of any publication cited herein is incorporated by reference.

In general, the compositions of the disclosure may be alternately formulated to comprise, consist of, or consist essentially of, any appropriate components herein disclosed. The compositions of the disclosure may additionally, or alternatively, be formulated so as to be devoid, or substantially free, of any components, materials, ingredients, adjuvants or species used in the prior art compositions or that are otherwise not necessary to the achievement of the function and/or objectives of the present disclosure.

The compounds of the present disclosure may have unnatural ratios of atomic isotopes at one or more of their atoms. For example, the compounds may be labeled with isotopes, such as deuterium (see for example compounds 3.4, 3.5, 4.4, and 4.5), tritium carbon-11, carbon-14, iodine-123, iodine-125 or fluorine-18. The present disclosure encompasses all isotopic variations of the described compounds, whether radioactive or not.

Testing of some compounds disclosed herein showed ability to activate angiogenic responses. Thus another aspect of the invention is use of at least one compound, and pharmaceutically acceptable salts thereof, to activate angiogenic responses.

Testing of some compounds disclosed herein showed ability to bind to, and modulate, (activate or deactivate) the putative “non-CB1/non-CB2 cannabinoid receptor”. Thus, another aspect of the invention is use of at least one compound, and pharmaceutically acceptable salts thereof, to bind to and modulate the putative “non-CB1/non-CB2 cannabinoid receptor”.

Testing of some compounds disclosed herein for their binding affinities for the central (CB1) and the peripheral (CB2) cannabinoid receptor, showed very low affinity for the two cannabinoid receptors. Therefore, another aspect of the invention is use of at least one of the inventive compounds, and physiologically acceptable salts thereof, to modulate the non-CB1/non-CB2 cannabinoid receptor.

The disclosed compounds and, pharmaceutically acceptable salts thereof, have high potential to be used as research tools. For example, labeled and unlabelled ligands (for example agonists and antagonists) that are specific for an as-yet-unidentified cannabinoid receptor distinct from CB1 and CB2 may be used to characterize and isolate the as-yet-unidentified cannabinoid receptor distinct from CB1 and CB2 that for example, regulate mesenteric vasodilation in animals and/or promote angiogenic responses. The disclosed compounds can be used as in vivo imaging agents and also they can aid in drug design, for example as a control in assays for testing other compounds for their ability to bind to a non-CB1/non-CB2 cannabinoid receptor and to determine the structure activity of non-CB1/non-CB2 cannabinoid ligands.

Analogs described herein, and physiologically acceptable salts thereof, when administered in therapeutically effective amounts, have high potential to bind to and modulate (activate or deactivate) the “non-CB1/non-CB2 endothelial cannabinoid receptor” and thereby provide a physiological effect in an individual or animal that is useful to treat a condition in that individual or animal. Conditions that may be treated by modulation of the “non-CB1/non-CB2 endothelial cannabinoid receptor” include for example: high blood pressure disease or hypertension; peripheral vascular disease; coronary artery disease; abnormal heart rate; pulmonary hypertension; ocular hypertension or glaucoma; diseases where hypotension is the result of the action of endogenous cannabinoids and drug-induced vasoconstriction is desirable, for example in hypotensive states, such as shock; vasodilatory shock (caused by vascular dilation, as seen for example in cerebral trauma, drug intoxication, heat exposure or septic shock accompanying a gram negative bacterial infection); cardiogenic shock (for example from arrhythmia or heart failure); to achieve selective hemostasis to stop bleeding induced by trauma or surgery; to treat angiogenesis-dependent events involved both in physiological and pathological conditions such as wound healing, placental development, stroke related blockage of blood capillaries, rheumatoid arthritis, diabetic retinopathy and tumor growth. Thus, another aspect of the disclosure is the administration of a therapeutically effective amount of a described compound, or a pharmaceutically acceptable salt thereof, to an individual or animal to provide a physiological effect for treatment of a condition in that individual or animal.

The disclosed compounds, and pharmaceutically acceptable salts thereof may be used to prepare prodrugs. As used herein, the term “prodrug” refers to any derivative of the compounds of general formula I, II and III that are metabolized or otherwise converted into an active form upon introduction into the body of an individual or animal. Prodrugs are well known to those skilled in the art of pharmaceutical chemistry, and provide benefits such as increased adsorption and half-life. Those skilled in the art of drug delivery will readily appreciate that the pharmacokinetic properties of general formulas I, II and III may be controlled by an appropriate choice of moieties to produce prodrug derivatives.

One or more disclosed compounds, typically after purification, can be incorporated into a pharmaceutical composition or medicament. The disclosed compounds can be administered by a variety of known methods, including, for example, orally, rectally, or by parenteral routes (e.g., intramuscular, intravenous, subcutaneous, nasal or topical). The form in which the compounds are administered will be determined by the route of administration. Such forms include, but are not limited to, capsular and tablet formulations (for oral and rectal administration), liquid formulations (for oral, intravenous, intramuscular, subcutaneous, ocular, intranasal, inhalation-based and transdermal administration) and slow releasing microcarriers (for rectal, intramuscular or intravenous administration). The pharmaceutical composition or medicament can also contain a pharmaceutically acceptable vehicle, diluent, excipient or carrier and optional adjuvants, flavorings, colorants, wetting agents, emulsifying agents, pH buffering agents and preservatives. Some suitable pharmaceutically acceptable vehicles include, for example, saline, sterile water, Ringer's solution and isotonic sodium chloride solutions. The specific dosage level of active ingredient will depend upon a number of factors, including, for example, biological activity of the particular preparation, age, body weight, sex and general health of the individual being treated.

Some disclosed compounds were tested using in vitro angiogenesis assays (cord formation by endothelial cells in extracellular matrix mimicking matrigel, Table 4), ex vivo angiogenesis assay (sprouting or blood capillary formation under ex vivo conditions, Table 4) as well as for MMP (matrix metalloproteinase) activity in CB1 receptor knock out human umbilical vein endothelial cells (HUVEC) (CB1-KO-HUVEC, Figures A and B). Stimulation of angiogenic responses under in vitro and ex vivo conditions as well as increasing in MMP activity, correlates well with the ability of the compound to bind to and modulate the putative “non CB1/non CB2 anandamide receptor”. For example compound 3.2 with an angiogenic score of 0.5 (at 1×10⁻⁶M) (see Table 4) exhibits lower binding affinity for the putative “non CB1/non CB2 anandamide receptor”, when compared with compound 3.15 which has an angiogenic score of 2.0 (at 1x10⁻⁶M) (see Table 4); or for example compounds 5.3, 3.6, 3.15, 4.11, 8.2 and 3.8 (see FIGS. 7 and 8) induce MMP activity with a higher potency (EC50˜50-70 nM) than (R)-methanandamide (EC50˜42 nM) and thus, they have higher binding affinities for the putative “non CB1/non CB2 anandamide receptor” when compared with (R)-methanandamide. A detailed description of the angiogenesis and MMP activity assays is given in Mukhopadhyay (US 2007, 0244200, and Balas, et al., J. Med. Chem. (2009) ASAP the content of each of which is hereby incorporated by reference.

Some disclosed compounds were tested for CB1 and CB2 receptors binding affinity (Table 5). As used herein “binding affinity” is represented by the Ki value. The Ki value is the affinity constant and describes the affinity of the compound for the receptor. The lower the Ki value, the higher the affinity of the compound for the receptor. A detailed description of the methods used to test “binding affinity” of compounds is given in Makriyannis, et al., US 2007, 0135388 and in Papahatjis, et al. J. Med. Chem. (2007) 4048, the content of each of which is hereby incorporated by reference.

The disclosed compounds and pharmaceutically acceptable salts thereof, have high potential to be used as research tools. For example, labeled and unlabelled ligands (either agonists and antagonists) that are specific for an as-yet-unidentified cannabinoid receptor distinct from CB1 and CB2 may be used to characterize and isolate the as-yet-unidentified cannabinoid receptor distinct from CB1 and CB2 that for example, regulate mesenteric vasodilation in animals and/or promote angiogenic responses. The disclosed compounds can be used as in vivo imaging agents and also they can aid in drug design, for example as a control in assays for testing other compounds for their ability to bind to a non-CB1/non-CB2 cannabinoid receptor and to determine the structure activity of non-CB1/non-CB2 cannabinoid ligands.

The invented compounds and physiologically acceptable salts thereof, when administered in therapeutically effective amounts, have high potential to bind to and modulate the “non-CB1/non-CB2 endothelial cannabinoid receptor” and thereby provide a physiological effect in an individual or animal that is useful to treat a condition in that individual or animal. Conditions that may be treated by modulation of the “non-CB1/non-CB2 endothelial cannabinoid receptor” include for example: high blood pressure associated diseases or hypertension; peripheral vascular disease; coronary artery disease; abnormal heart rate; pulmonary hypertension; ocular hypertension or glaucoma; diseases where hypotension is the result of the action of endogenous cannabinoids and drug-induced vasoconstriction is desirable, for example in hypotensive states, such as shock; vasodilatory shock (caused by vascular dilation, as seen for example in cerebral trauma, drug intoxication, heat exposure or septic shock accompanying a gram negative bacterial infection); cardiogenic shock (for example from arrhythmia or heart failure); to achieve selective hemostasis to stop bleeding induced by trauma or surgery; to treat angiogenesis-dependent events involved both in physiological and pathological conditions such as wound healing, placental development, stroke related blockage of blood capillaries, rheumatoid arthritis, diabetic retinopathy and tumor growth.

The invention is further described in the following examples, which do not limit the scope of the invention described in the claims.

### EXAMPLES

### Synthesis of Compounds I, IL and III

Synthesized compounds represented by the general formula I, II and III are depicted in Tables 1, 2 and 3 respectively, on the following pages.

TABLE 1
Compounds of the general formula I.

| Compound | |
|---|---|
| Number | Structure |
| 3.1 | 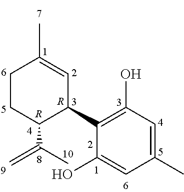 |
| 3.2 | 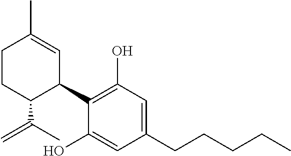 |
| 3.3 | 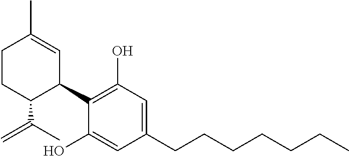 |
| 3.4 | 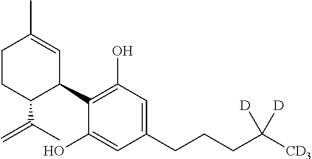 |
| 3.5 | 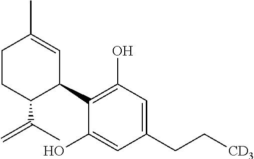 |
| 3.6 | 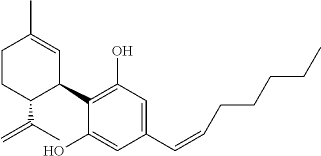 |
| 3.7 | 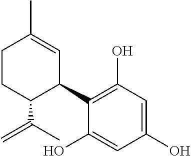 |
| 3.8 | 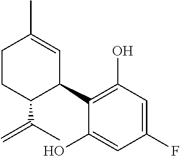 |
| 3.11 | 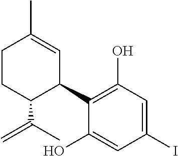 |
| 3.12 | 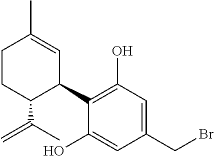 |
| 3.13 | 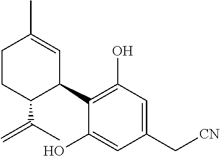 |
| 3.14 | 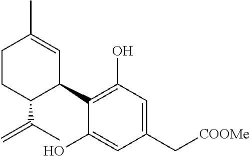 |
| 3.15 | 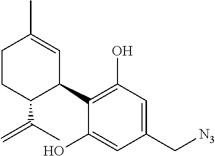 |
| 3.16 | 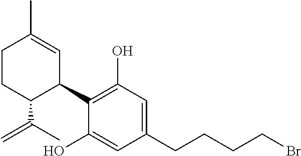 |
| 8.2 | 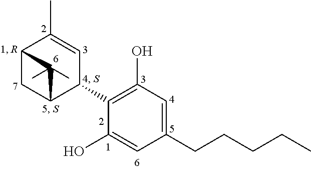 |
| 11.2 | 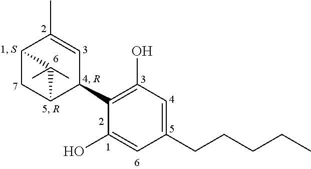 |

TABLE 2
Compounds of the general formula II.

| Compound | |
|---|---|
| Number | Structure |
| 4.1 | 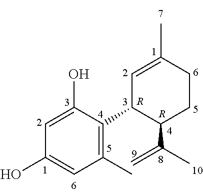 |
| 4.2 | 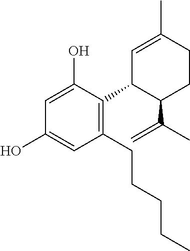 |
| 4.3 | 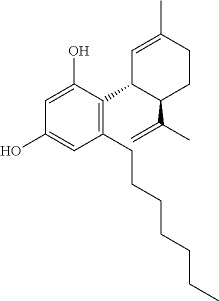 |
| 4.4 | 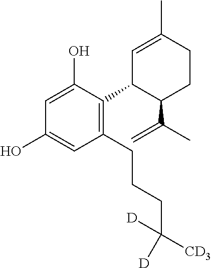 |
| 4.5 | 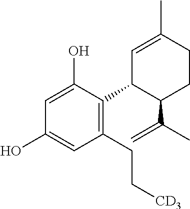 |
| 4.6 | 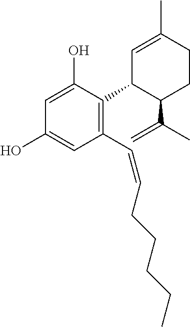 |
| 4.8 | 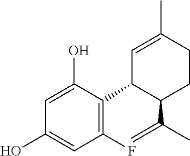 |
| 4.9 | 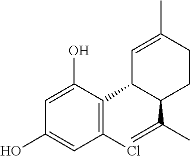 |
| 4.10 | 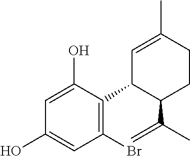 |
| 4.11 | 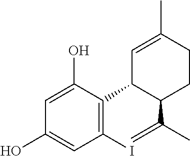 |
| 4.12 | 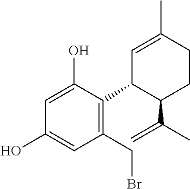 |
| 4.13 | 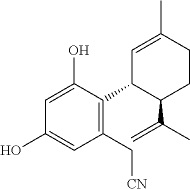 |
| 4.14 | 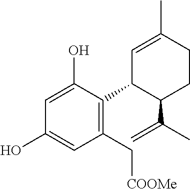 |
| 4.15 | 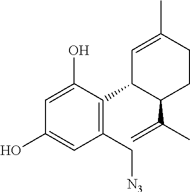 |
| 4.16 | 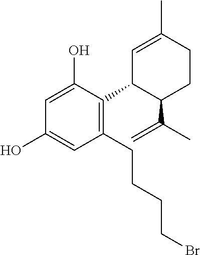 |
| 9.2 | 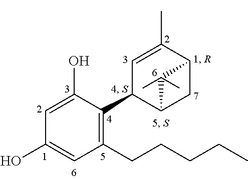 |
| 12.2 | 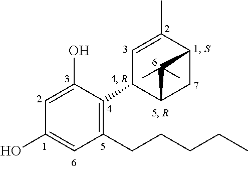 |
| 14.2 | 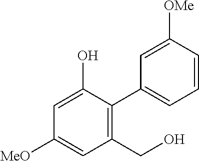 |

TABLE 3
Compounds of the general formula III.

| Compound | |
|---|---|
| Number | Structure |
| 5.3 | 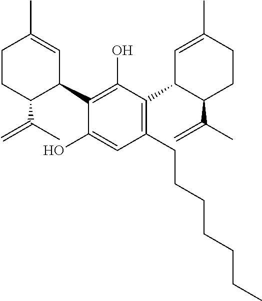 |
| 5.14 | 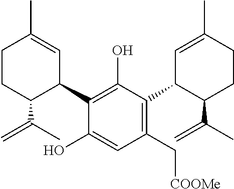 |
| 6.10 | 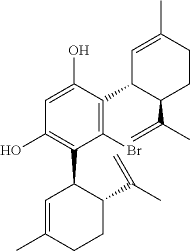 |
| 6.11 | 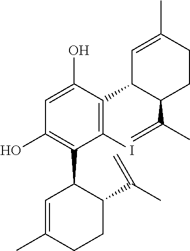 |

A. Resorcinol Synthesis

Resorcinol compounds 1.1, 1.2 and 1.7 (shown in Scheme 7) were commercially available.

Resorcinol compound 1.3 (shown in Scheme 7) was synthesized in three steps starting from commercially available 3,5-dimethoxybenzaldehyde, by a method disclosed in Papahatjis, et al. J. Med. Chem. (2007) 50:4048-4060, the content of which is hereby incorporated by reference.

Resorcinol compounds 1.4 and 1.5 (shown in Scheme 7) were synthesized in eight and three steps respectively, starting from commercially available 3,5-dimethoxybenzaldehyde and following methods disclosed in Nikas, et al. J. Chem. Soc., Perkin Trans, 1 (2002) 2544-2548, in Nikas, et al. Synth. Commun. (2002) 32:1751-1756 and in Nikas, et al., J. Labelled Compd. Radiopharm. (2002) 1065-1076, the content of which is hereby incorporated by reference. compound 1.16 (shown in Scheme 7) was synthesized in six steps starting from commercially available 3,5-dimethoxybenzaldehyde by a method disclosed in Nikas, S. P. AAPSJ, 6, e30 (2004), (http://www.aapsj.org), the content of which is hereby incorporated by reference.

Resorcinol compound 1.6 (shown in Scheme 1) was synthesized by a method depicted in Scheme 1 starting from a Z/E mixture of alkenes 16 which was in turn prepared by a method disclosed in Papahatjis, et al. J. Med. Chem. (2007) 50:4048-4060, the content of which is hereby incorporated by reference.

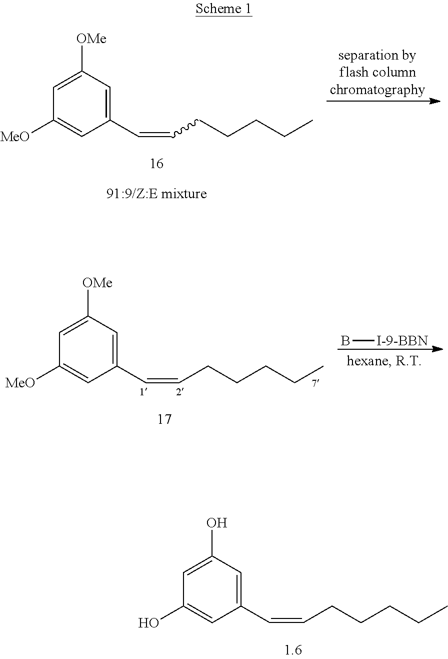

### Experimental Procedures:

### 1. 1-(1,2-cis-Hepten-1-yl)-3,5-dimethoxybenzene (Compound 17)

The title compound was separated from its mixture with the trans-isomer by flash column chromatography on silica gel. Yield: 77%; colorless liquid. ¹H NMR (500 MHz, CDCl₃) δ: 6.43 (d, J=2.2 Hz, 2H, ArH), 6.35 (t, J=2.2 Hz, 1H, ArH), 6.33 (d, J=11.5 Hz, 1H, 1′-H), 5.65 (dt, J=11.5, J=7.0 Hz, 1H, 2′-H), 3.79 (s, 6H, OMe), 2.32 (qd, J=7.3 Hz, J=1.7 Hz, 2H, 3′-CH₂—), 1.46 (quintet, J=7.3 Hz, 2H, 4′-CH₂—), 1.40-1.21 (m, 4H, 5′-CH₂—, 6′-CH₂—), 0.88 (t, J=7.1 Hz, 3H, 7′-CH₃).

### 2. 5-(1,2-cis-Hepten-1-yl)resorcinol (Compound 1.6)

To a solution of 17 (432 mg, 1.85 mmol) in anhydrous hexane (30 mL) at 0° C. under an argon atmosphere, was added B-1-9-borabicyclo[3.3.1]nonane (6.5 mL, 1M solution in hexanes). The mixture was warmed to room temperature and stirred until the reaction was completed (4 h). The reaction mixture was then concentrated and the residue was diluted with anhydrous diethyl ether (50 mL). A solution of ethanolamine (427 mg, 7 mmol) in anhydrous THF (12 mL) was added causing spontaneous precipitation of a white solid. The suspension was stirred for 1 h, the white solid was filtered off, and the filtrate was evaporated. Purification by flash column chromatography on silica gel (50% diethyl ether in hexane) gave 1.6 as a viscous oil in 89% yield (339 mg). ¹H NMR (500 MHz, CDCl₃) δ: 6.33 (d, J=2.1 Hz, 2H, ArH), 6.26 (d, J=11.7 Hz, 1H, 1′-H), 6.23 (t, J=2.1 Hz, 1H, ArH), 5.64 (dt, J=11.7, J=7.3 Hz, 1H, 2′-H), 4.78 (br s, 2H, OH), 2.31 (qd, J=7.2 Hz, J=1.7 Hz, 2H, 3′-CH₂—), 1.42 (quintet, J=7.2 Hz, 2H, 4′-CH₂—), 1.37-1.23 (m, 4H, 5′-CH₂—, 6′-CH₂—), 0.88 (t, J=7.0 Hz, 3H, 7′-CH₃).

Resorcinol compounds 1.8 and 1.9 (shown in Scheme 2) were synthesized by a method depicted in Scheme 2, starting from commercially available resorcinol dimethyl ethers 18.8 and 18.9.

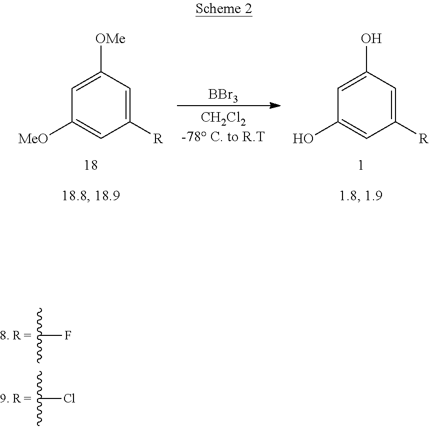

### Experimental Procedures:

To a stirred solution of resorcinol dimethyl ether 18 (1 equiv.) in dry methylene chloride (0.1 M) at −78° C. under an argon atmosphere was added boron tribromide (2.3 equiv., 1M solution in methylene chloride). Following the addition, the reaction temperature was gradually raised over a period of 1 h to room temperature and stirring was continued until completion of the reaction. Unreacted boron tribromide was destroyed by adding methanol and ice at 0° C. The mixture was warmed to room temperature, stirred for 40 min and concentrated in vacuo. The residue was diluted with ethyl acetate and washed with saturated sodium bicarbonate solution, water and brine. The organic layer was dried (MgSO₄) and concentrated under reduced pressure. Purification by flash column chromatography on silica gel afforded compound 1.

### 1. 5-Fluororesorcinol (Compound 1.8)

Yield: 92%; white solid; m p=133-135° C. ¹H NMR (200 MHz, CDCl₃+DMSO-d₆) δ: 8.80-6.52 (br s, 2H, OH), 6.25-6.05 (m, 3H, ArH).

### 5-Chlororesorcinol (Compound 1.9)

Yield: 96%; yellow solid; m p=113-115° C. ¹H NMR (200 MHz, CDCl₃+DMSO-d₆) δ: 8.40 (br s, 2H, OH), 6.36 (d, J=2.2 Hz, 2H), 6.26 (t, J=2.2 Hz, 1H).

Resorcinol compounds 1.10 and 1.11 (shown in Scheme 3) were synthesized by a method depicted in Scheme 3, starting from commercially available 3,5-dimethoxybenzoyl chloride (19).

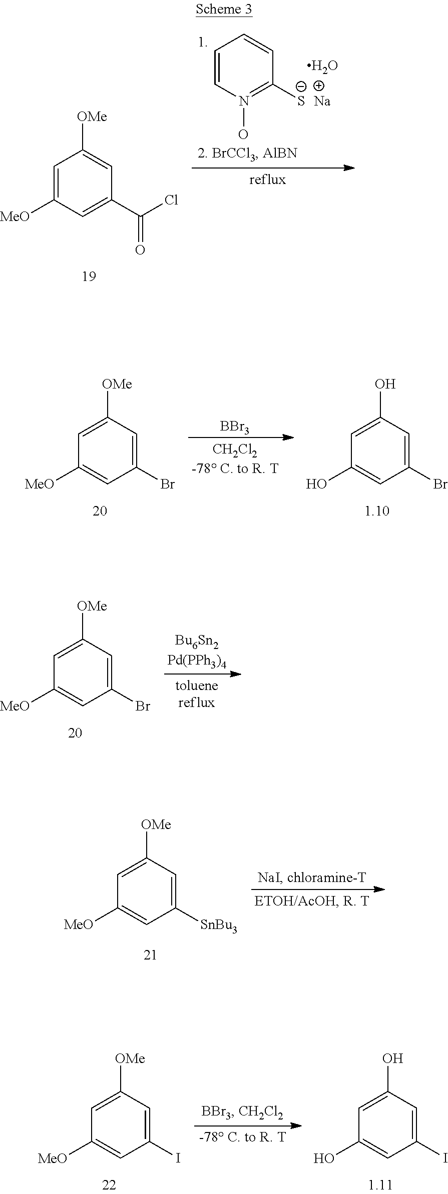

### Experimental Procedures:

### 1. 1-Bromo-3,5-dimethoxybenzene (Compound 20)

To a refluxing suspension of 2-mercaptopyridine N-oxide sodium salt (9.2 g, 55 mmol) in CBrCl₃ (105 ml), under an argon atmosphere was added a mixture of 3,5-dimethoxybenzoyl chloride (10 g, 50 mmol) and AIBN [2,2′-azobis(2-methylpropionitrile)] (1.23 g, 7.5 mmol) in CBrCl₃ (100 ml), over a period of 40 min. Reflux was continued for 15 min and then the reaction mixture was cooled to room temperature and filtered through a short pad of Celite. The filtrate was concentrated under reduced pressure and the residue was purified by flash column chromatography on silica gel (10% diethyl ether in hexane) to give the title compound (8.36 g, 77% yield) as a white solid. m p=65-66° C. ¹H NMR (200 MHz, CDCl₃) δ: 6.67 (d, J=1.9 Hz, 2H), 6.39 (t, J=1.9 Hz, 1H), 3.77 (s, 6H, OMe).

### 2. 5-Bromoresorcinol (Compound 1.10)

The synthesis was carried out analogous to the preparation of compounds 1.8 and 1.9 (Scheme 2). Yield: 79%; brown solid; m p=85-86° C. ¹H NMR (200 MHz, CDCl₃+DMSO-d₆) δ: 6.60 (d, J=1.6 Hz, 2H), 6.29 (t, J=1.6 Hz, 1H), 5.46 (br s, 2H, OH).

### 1- Tri-n-butylstannyl-3,5-dimethoxybenzene (Compound 21)

1-Bromo-3,5-dimethoxybenzene (5 g, 23 mmol) was dissolved in anhydrous toluene (115 ml) and the solution was degassed by bubbling argon. Hexabutylditin (33.6 g, 58 mmol) was added via syringe, followed by addition of tetrakis(triphenylphosphine)palladium (797 mg, 0.69 mmol). Following these additions the reaction mixture was refluxed for 4 h. After removal of the solvent under reduced pressure, the crude product was applied to a silica gel flash chromatographic column. The excess hexabutylditin was eluted first using 2% diethyl ether in hexane and the title compound was isolated from the column as a colorless oil (7.78 g, 79% yield) by elution with 5% diethyl ether in hexane. ¹H NMR (200 MHz, CDCl₃) δ: 6.61 (br s, 2H, 2-H, 6-H), 6.40 (br s, 1H, 4-H), 3.80 (s, 6H, OMe), 1.65-1.44 (m, 6H), 1.42-1.21 (m, 6H), 1.04 (t, J=7.9 Hz, 6H), 0.88 (t, J=7.1 Hz, 9H).

### 3. 1-Iodo-3,5-dimethoxybenzene (Compound 22)

To a solution of 1-tri-n-butylstannyl-3,5-dimethoxybenzene (5.5 g, 12.9 mmol) in ethanol (120 ml) and acetic acid (60 ml), was added sodium iodide (7.74 g, 51.6 mmol), followed by chloramine-T trihydrate (5.46 g, 19.4 mmol). The reaction mixture was stirred at room temperature for 45 min and then quenched by adding 5% sodium metabisulphite solution. The mixture was diluted with diethyl ether and water, the organic layer was separated and the aqueous phase extracted with diethyl ether. The combined organic layer was washed with saturated sodium bicarbonate solution and brine, dried (MgSO₄) and evaporated. The residue was purified by flash column chromatography on silica gel (5% diethyl ether in hexane) to give the title compound as white solid in 92% yield (3.13 g); m p=72-74° C. ¹H NMR (200 MHz, CDCl₃) δ: 6.86 (d, J=2.2 Hz, 2H), 6.40 (t, J=2.2 Hz, 1H), 3.76 (s, 6H, OMe).

### 4. 5-Iodoresorcinol (Compound 1.11)

The synthesis was carried out analogous to the preparation of compounds 1.8 and 1.9 (Scheme 2) starting from 3.0 g (11.36 mmol) of 1-iodo-3,5-dimethoxybenzene. Yield: 96% (2.57 g); white solid; m p=83-85° C. ¹H NMR (200 MHz, CDCl₃+DMSO-d₆) δ: 8.49 (br s, 2H, OH), 6.74 (d, J=2.3 Hz, 2H), 6.35 (t, J=2.3 Hz, 1H).

Resorcinol compounds 1.12, 1.13 and 1.14 (shown in Scheme 4) were synthesized by a method depicted in Scheme 4, starting from commercially available 3,5-dimethoxybenzylbromide (23.12) and 3,5-dimethoxybenzylcyanide (23.13).

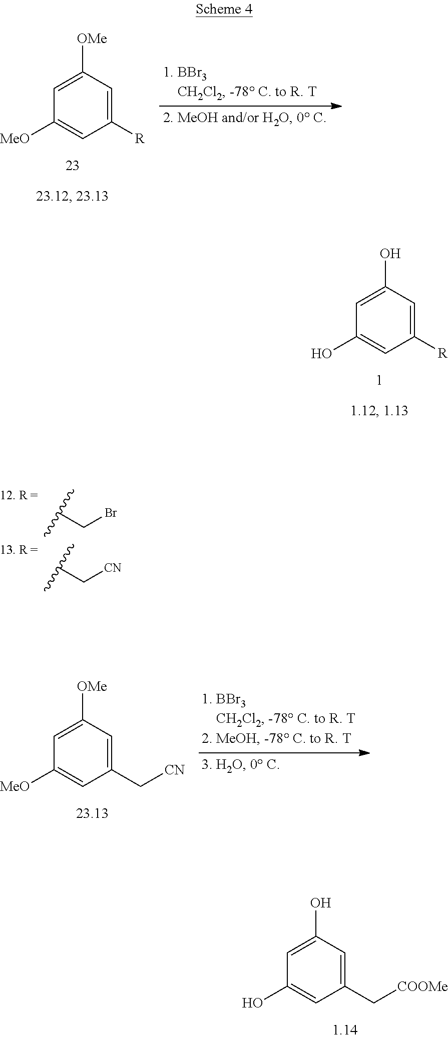

### Experimental Procedures:

### 1. 5-(Bromomethyl)resorcinol (Compound 1.12)

The synthesis was carried out analogous to the preparation of compounds 1.8 and 1.9 (Scheme 2). Yield 84%; ¹H NMR (500 MHz, CDCl₃+DMSO-d₆) δ: 7.95 (br s, 2H, OH), 6.42 (d, J=2.0 Hz, 2H), 6.34 (t, J=2.0 Hz, 1H), 4.35 (s, 2H, —CH₂Br).

### 2. 5-(Cyanomethyl)resorcinol (Compound 1.13)

To a stirred solution of 3,5-dimethoxybenzyl cyanide (1 g, 5.65 mmol) in dry methylene chloride (37 ml) at −78° C. under an argon atmosphere was added boron tribromide (19 ml, 1M solution in methylene chloride). The reaction temperature was gradually raised over a period of 20 min to room temperature and stirring was continued for 7 hours. The reaction was quenched by dropwise addition of water at 0° C. and volatiles were removed under reduced pressure. The residue was dissolved in diethyl ether and the solution was washed with saturated sodium bicarbonate solution, water and brine. The organic layer was dried (MgSO₄) and concentrated in vacuo. Purification by flash column chromatography on silica gel (50% ethyl acetate in hexane) gave compound 1.13 (530 mg, 63% yield). IR (neat) 3357, 3212, 2272 (w, —C≡N ), 1625, 1604, 1507, 1486, 1409, 1334, 1163, 1150, 1009, 986, 920, 844, 814 cm⁻¹; ¹H NMR (500 MHz, CDCl₃+DMSO-d₆) δ: 8.77 (br s, 2H, OH), 6.34 (t, J=2.0 Hz, 1H), 6.32 (d, J=2.0 Hz, 2H), 3.60 (s, 2H, —CH₂CN).

### 3. 5-(2-Methoxy-2-oxoethyl)resorcinol (Compound 1.14)

To a stirred solution of 3,5-dimethoxybenzyl cyanide (5 g, 28.2 mmol) in dry methylene chloride (188 ml) at −78° C. under an argon atmosphere was added boron tribromide (93 ml, 1M solution in methylene chloride). The reaction temperature was gradually raised over a period of 3 h to room temperature, and stirring was continued for 24 hours. The reaction mixture was cooled to −78° C. and methanol (20 ml) was added. Following this addition, the mixture was warmed to room temperature and quenched by dropwise addition of water at 0° C. The organic phase was separated and the aqueous phase was extracted with diethyl ether. The combined organic layer was washed with 2N NaOH solution, water and brine, and dried over MgSO₄. Solvent evaporation and purification by flash column chromatography on silica gel (50% ethyl acetate in hexane) gave compound 1.14 (2.3 g, 45% yield). Viscous oil; IR (neat) 3383, 3318, 2966, 2916, 1707 (s, >C═O), 1634, 1593, 1496, 1437, 1376, 1336, 1300, 1216, 1146, 1008, 965, 832 cm⁻¹; ¹H NMR (500 MHz, CDCl₃+DMSO-d₆) δ: 7.82 (s, 2H, OH), 6.31 (s, 3H, ArH), 3.67 (s, 3H, —CH₂COOCH₃), 3.47 (s, 2H, —CH₂COOCH₃).

B. Chiral Monoterpenoid Alcohol Synthesis

(+)-cis/trans-p-Mentha-2,8-dien-1-ol (compound 2, shown in Scheme 7) was commercially available (Firmenich Inc. Princeton, N.J.) while optically pure (1R, 4R, 5R)-2-pinene-4-ol ((+)-cis-verbenol, compound 7) was synthesized by a reaction sequence shown in Scheme 5 starting from commercially available (1R)-(+)-α-pinene (24, 96-97% ee).

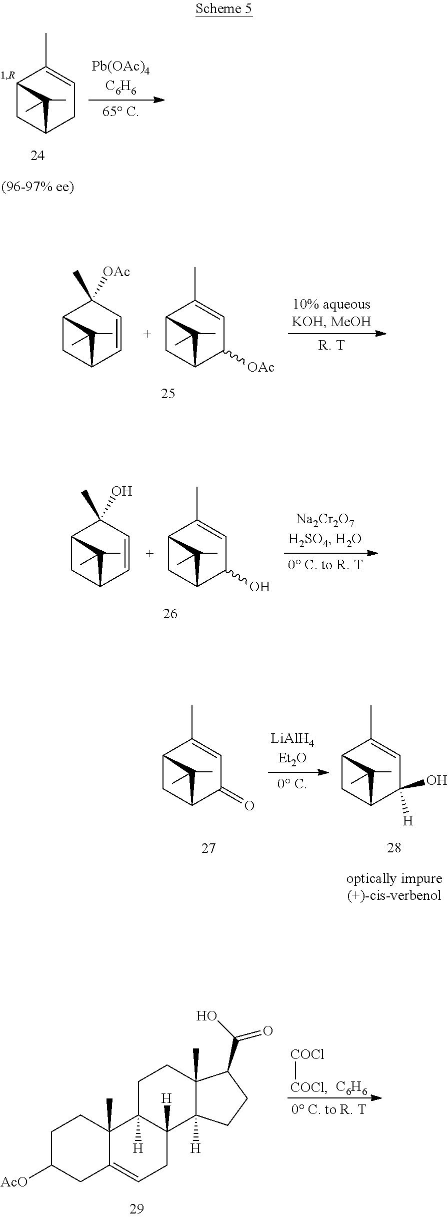

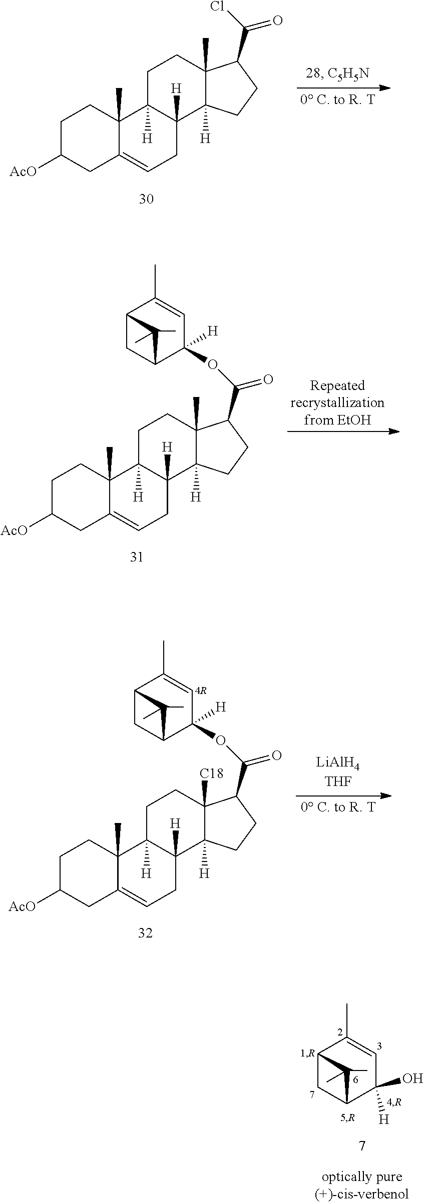

### Experimental Procedures:

### 1. Optically Impure (1R, 5R)-(+)-Verbenone (Compound 27)

To a heated (65° C.) and stirred solution of (1R)-(+)-α-pinene (96-97% ee, 52 g, 382 mmol) in anhydrous benzene (640 ml) under an argon atmosphere, was added lead(IV)acetate (163 g, 367 mmol, previously dried in vacuo) over a period of 15 min. Stirring and heating at 65° C. were continued for 2 hours and then the reaction mixture was cooled to room temperature and filtered through a short pad of Celite. The Celite pad was washed with several portions of diethyl ether. To the filtrate was added water (400 ml, formation of brown-black lead oxide), the mixture was stirred for 20 min and filtered through a pad of Celite. The organic phase was separated, the aqueous phase was extracted with diethyl ether and the combined organic layer was washed with brine and dried (MgSO₄). Solvent evaporation under reduced pressure gave a crude mixture of acetates 25 (68 g) as a colorless liquid which was used into the next step without further purification.

To the mixture of acetates 25 (68 g) at room temperature, was added a 10% solution of KOH (29.4 g, 525 mmol) in Me0H (294 ml)/H₂O (17 ml) over a period of 1 hour. The reaction mixture was stirred for 24 h, then diluted with water and extracted with diethyl ether. The organic layer was dried over MgSO₄ and evaporated under reduced pressure (30° C.) to leave a crude mixture of alcohols 26 (98 g) as a yellow oil which was used into the next step as such.

To a stirred solution of alcohols 26 (98 g) in diethyl ether (600 ml) at 0° C. was added, over a period of 40 min, a mixture of sodium dichromate dihydrade (137 g, 461 mmol), water (500 ml) and concentrated H₂SO₄ (53 ml). Stirring was continued at 0° C. for 1 h and at room temperature for 12 h. The reaction mixture was diluted with water, the organic phase was separated, and the aqueous layer was extracted with diethyl ether. The combined organic layer was washed with saturated NaHCO₃ solution and brine, and dried over MgSO₄. Solvent evaporation and purification by flash column chromatography on silica gel (25-35% diethyl ether in hexane) afforded 24.7 g of compound 27 (43% overall yield) as a colorless liquid.

### Optically Impure (1R, 4R, 5R)-(+)-2-pinen-4-ol ((+)-cis-Verbenol, Compound 28)

To a stirred suspension of LAH (6.5 g, 169.9 mmol) in anhydrous diethyl ether (400 ml) at 0° C. under an argon atmosphere, was added a solution of verbenone (27, 19.6 g, 130.7 mmol) in anhydrous diethyl ether (250 ml) over a period of 10 min. The reaction mixture was stirred vigorously for 1 h at the same temperature and then quenched by adding NaF (7.1 g, 170 mmol) followed by dropwise addition of 10% aqueous NaOH. The mixture was then warmed to room temperature, diluted with diethyl ether and water, and stirred for 20 min. The suspension was filtered through a short pad of Celite and the organic layer was separated. The aqueous phase was extracted with diethyl ether and the combined organic layer was washed with brine and dried over MgSO₄. Solvent evaporation and purification by flash column chromatography on silica gel (35% diethyl ether in hexane) gave the title compound in 96% yield (19.1 g) as a white solid.

¹H NMR (500 MHz, CDCl₃) δ: 5.37 (br s, 1H, 3-H), 4.46 (br s, 1H, 4-H), 2.44 (ddd as dt, 1H, J=9.0 Hz, J=5.5 Hz, 7α-H), 2.32-2.27 (m, 1H, 5-H), 1.97 (t, 1H, J=5.5 Hz, 1-H), 1.73 (s, 3H, C10-CH₃), 1.35 (s, 3H, C8-CH₃), 1.30 (d, 1H, J=9.3 Hz, 7β-H), 1.08 (s, 3H, C9-CH₃).

### 2. 3β-acetoxyetienic ester of (+)-cis-verbenol (Compound 32)

To a stirred suspension of 3β-acetoxy-5-etienic acid (29, 20 g, 55.7 mmol) in anhydrous benzene (185 ml) under an argon atmosphere at 3° C., was added oxalyl chloride (214 ml, 2.0 M solution in CH₂Cl₂) over a period of 15 min. The reaction mixture was stirred for 20 min at 3° C. and for 40 min at room temperature. The pale yellow solution was concentrated in vacuo, the residue was dissolved in anhydrous benzene and the solvent evaporated under reduced pressure to leave the crystalline acyl chloride 30 (23.3 g) which was used into the next step without further purification.

To a stirred solution of the acyl chloride 30 in anhydrous pyridine (140 ml) at 0° C. under an argon atmosphere was added a solution of 28 (7.7 g, 50.6 mmol) in anhydrous pyridine (30 ml) over a period of 3 min. The reaction mixture was warmed to room temperature, stirred for 15 min and poured into ice-5% aqueous HCl. The mixture was extracted with AcOEt, insoluble materials were filtered off and the organic layer washed with 5% HCl, saturated NaHCO₃ solution and brine, and dried over MgSO₄. Solvent evaporation and purification by flash column chromatography on silica gel (15% diethyl ether in hexane) afforded 23.3 g (93% yield) of compound 31 as a white solid, m p=161-164° C. Compound 31 (20.6 g) was repeatedly recrystallized (three times) from ethyl alcohol to give 16.7 g (81%) of compound 32. m p=168-170° C.

¹H NMR (500 MHz, CDCl₃) δ: 5.56 (br s, 1H), 5.36 (d, J=4.6 Hz, 1H), 5.29 (br s, 1H), 4.64-4.55 (m, 1H), 2.48 (dt, J=9.0 Hz, J=6.0 Hz, 1H), 2.34-2.26 (m, 4H), 2.18-2.06 (m, 2H), 2.03-1.96 (m, 5H, especially 2.02, s, 3H, CH₃C(O)—), 1.90-1.84 (m, 2H), 1.83-1.75 (m, 1H), 1.74 (s, 3H, —C(CH₃)═C<), 1.72-1.65 (m, 1H), 1.64-1.54 (m, 3H), 1.52-1.42 (m, 2H overlapping with traces of H₂O), 1.40 (d, J=9.0 Hz, 1H), 1.34 (s, 3H, —C(CH₃)₂—), 1.30-1.22 (m, 2H), 1.18-1.05 (m, 2H), 1.03-0.95 (m, 7H, especially 1.02, br s, 6H, —C(CH₃)₂—+C19-CH₃), 0.71 (s, 3H, C18-CH₃).

Optically pure (1R, 4R, 5R)-(+)-2-pinen-4-ol ((+)-cis-Verbenol, compound 7). To a stirred suspension of LAH (10.4 g, 273 mmol) in anhydrous THF (200 ml) at 0° C. under an argon atmosphere was added a solution of 32 (15 g, 30.4 mmol) in anhydrous THF (100 ml) over a period of 1 h. The reaction mixture was warmed to room temperature and stirred vigorously for 1 h. Workup of the reaction was performed in the usual manner as described for compound 28. Purification by flash column chromatography on silica gel (40% diethyl ether in hexane) gave optically pure (+)-cis-verbenol (7) as a white solid in 93% yield (4.3 g), m p=73-73.5° C., [α]_{D}²⁶=+10.4° (c=0.47%, CHCl₃). The ¹H NMR spectrum was identical to that of the optically impure 28.

Similarly, optically pure (1S, 4S, 5S)-2-pinene-4-ol ((−)-cis-verbenol, compound 10) was synthesized by a reaction sequence shown in Scheme 6 starting from commercially available (1S)-(−)-α-pinene (33, 96-97% ee).

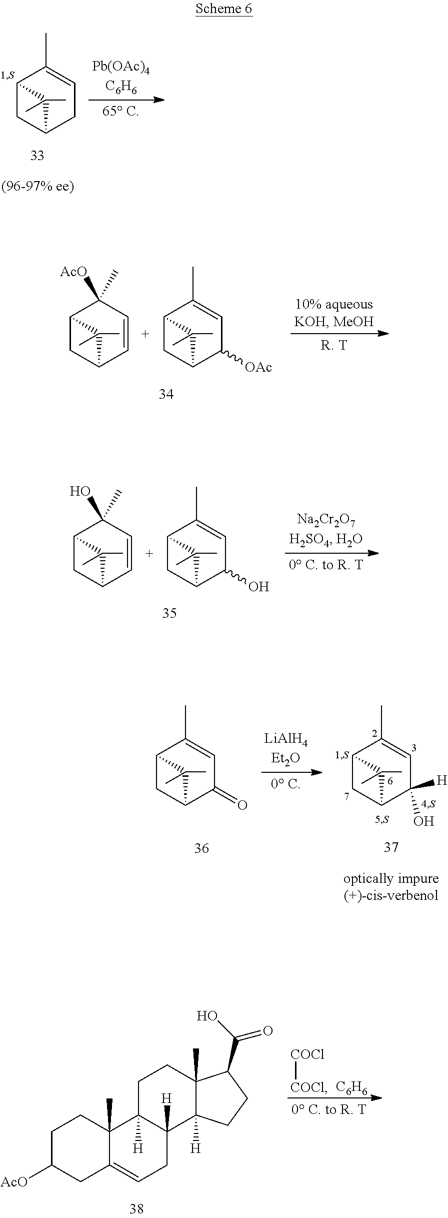

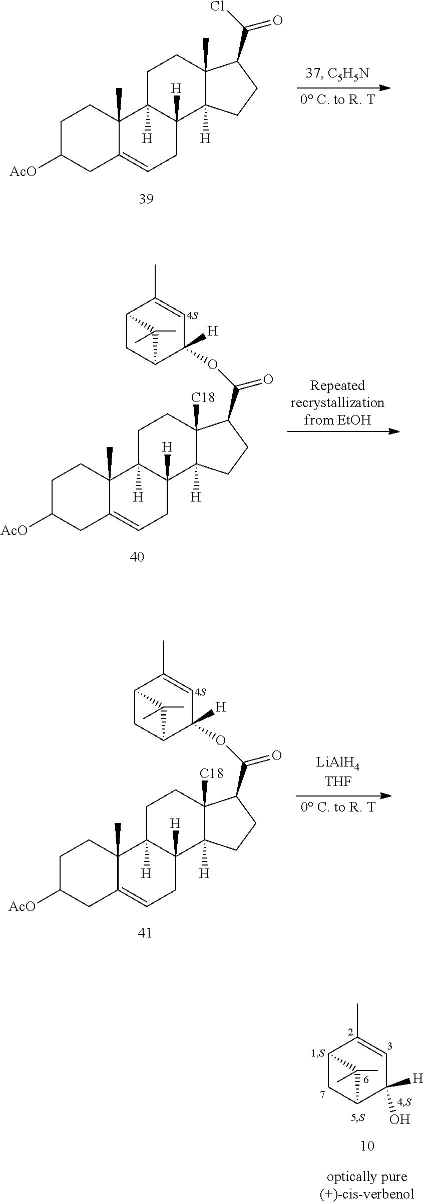

### Experimental Procedures:

The synthesis of 36, 37, 40, 41 and 10 was carried out analogous to the preparation of 27, 28, 31, 32, and 7 respectively. The ¹H NMR spectra of compounds 36, 37 and 10 were identical to those of the enantiomers 27, 28 and 7.

### 1. 313-Acetoxyetienic ester of (−)-cis-verbenol (Compound 41)

¹H NMR (500 MHz, CDCl₃) δ: 5.54 (br s, 1H), 5.37 (d, J=4.6 Hz, 1H), 5.33 (br s, 1H), 4.64-4.56 (m, 1H), 2.49 (dt, J=9.0 Hz, J=6.0 Hz, 1H), 2.36-2.25 (m, 4H), 2.14 (m as q, J=11.5 Hz, 1H), 2.09-1.96 (m, 6H, especially 2.03, s, 3H, CH₃C(O)—), 1.91-1.83 (m, 2H), 1.82-1.75 (m, 1H), 1.74-1.65 (m, 4H, especially 1.73, s, 3H, —C(CH₃)═C<), 1.64-1.53 (m, 3H overlapping with traces of H₂O), 1.52-1.39 (m, 3H), 1.35 (s, 3H, —C(CH₃)₂—), 1.31-1.21 (m, 2H), 1.19-1.05 (m, 2H), 1.05-0.95 (m, 7H, especially 1.02, br s, 6H, —C(CH₃)₂—+C19-CH₃), 0.69 (s, 3H, C18-CH₃).

### 2. Determination of the Enantiomeric Purity of Verbenols 7 and 10 by ¹H NMR

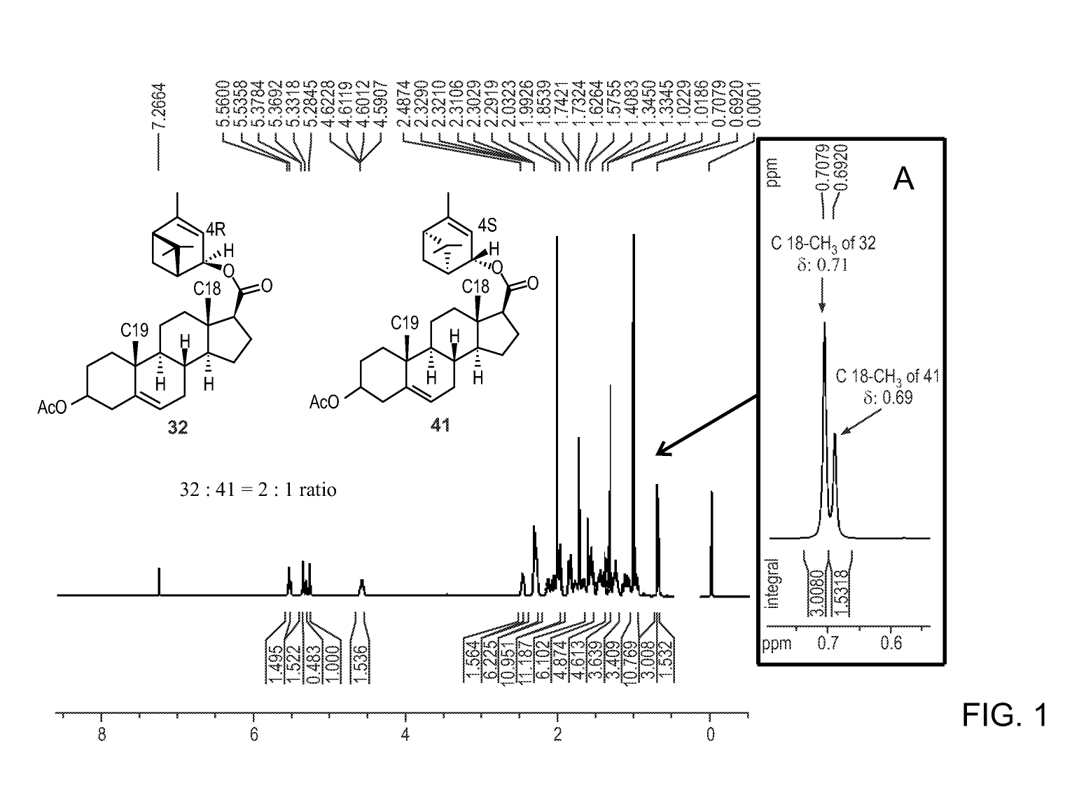

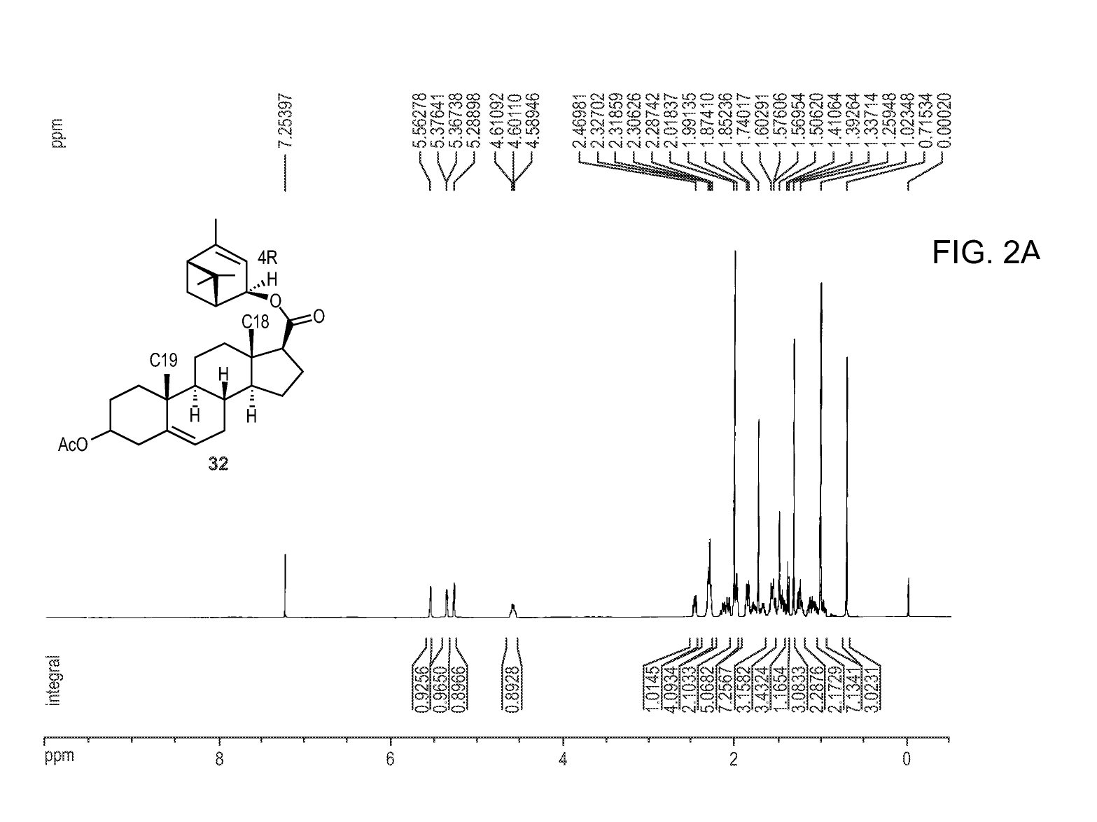

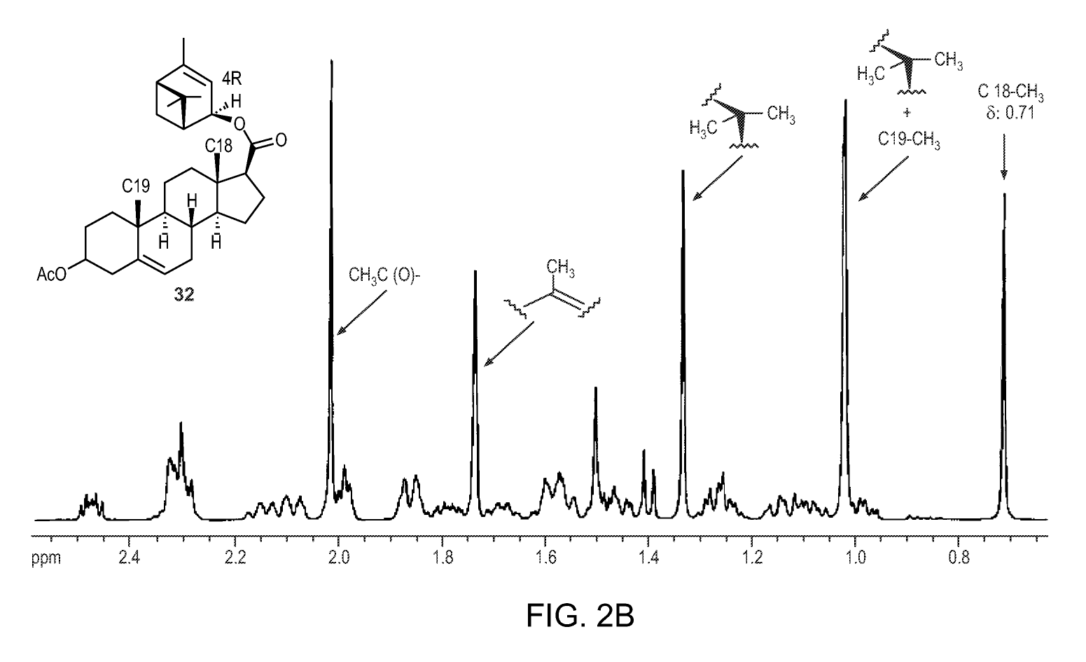

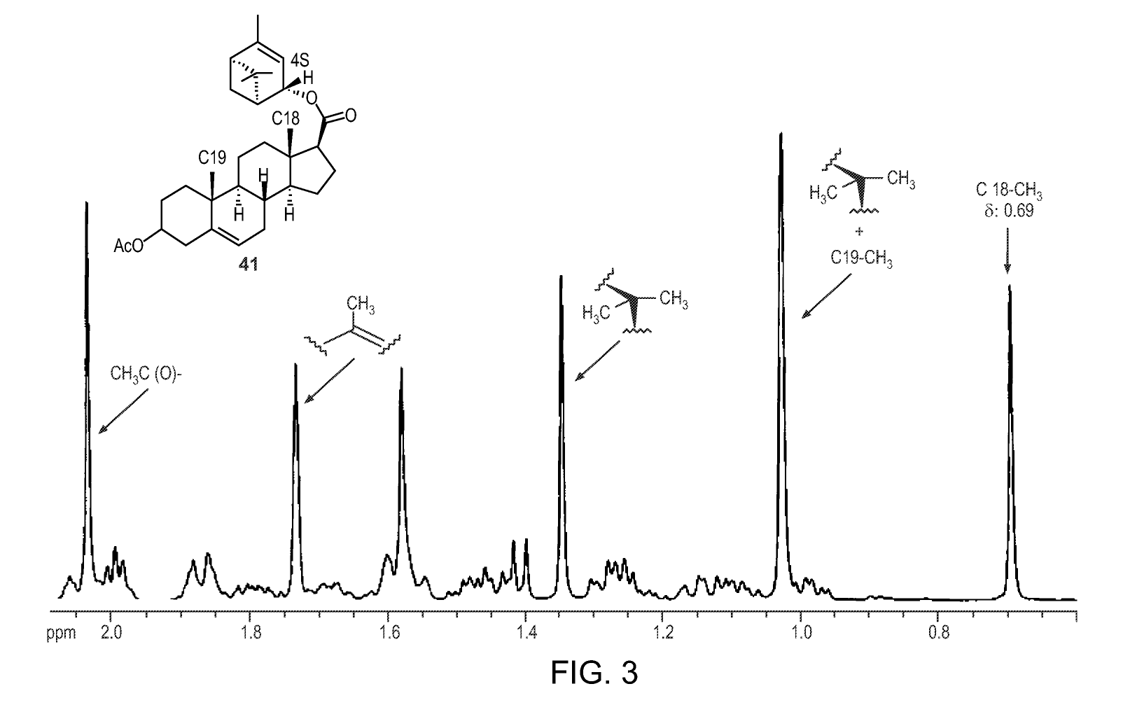

Our first approach in determining the enantiomeric purity of 7 and 10 utilizes the 1H NMR spectra of their respective diastereomers 32 and 41. The signals for the C18-CH3 protons of the steroid skeleton were differentiated (δ: 0.71 for 32 and δ: 0.69 for 41). This is shown in FIG. 1 where the two diastereomeric etianates 32 and 41 were mixed in a known ratio to display the separation of the peaks in a mixture situation. Using this method we did not observe any sign of cross-contamination of the two diastereomers (FIGS. 2 and 3). Therefore, the enantiomers 7 and 10 were synthesized in enantiomerically pure forms.

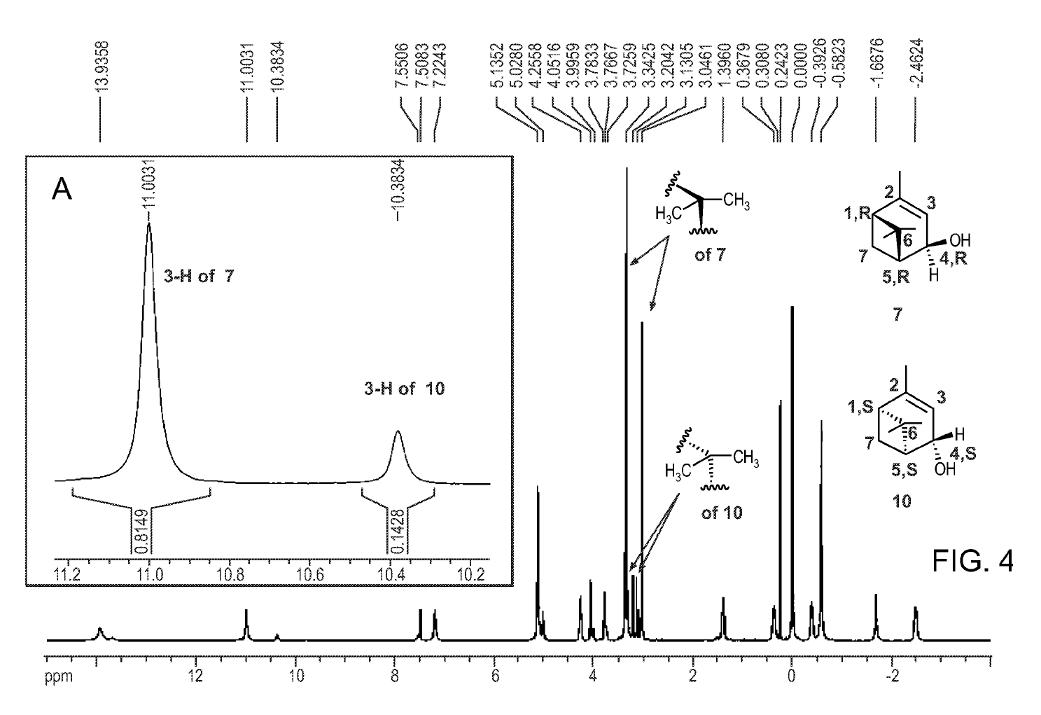

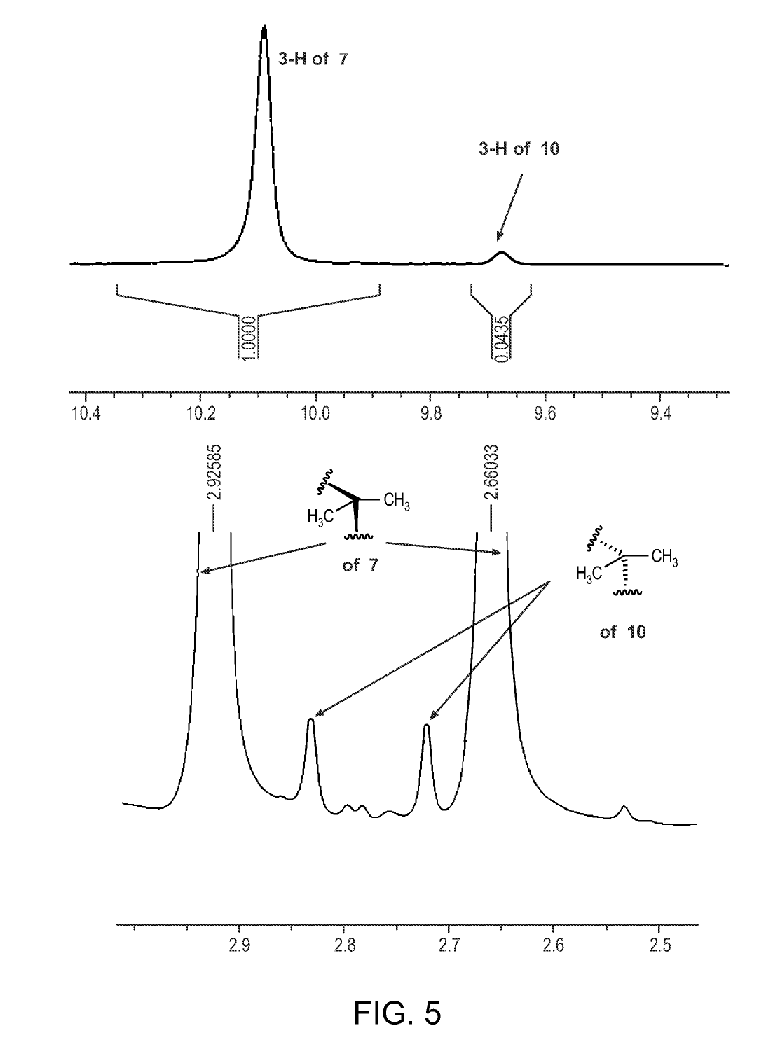

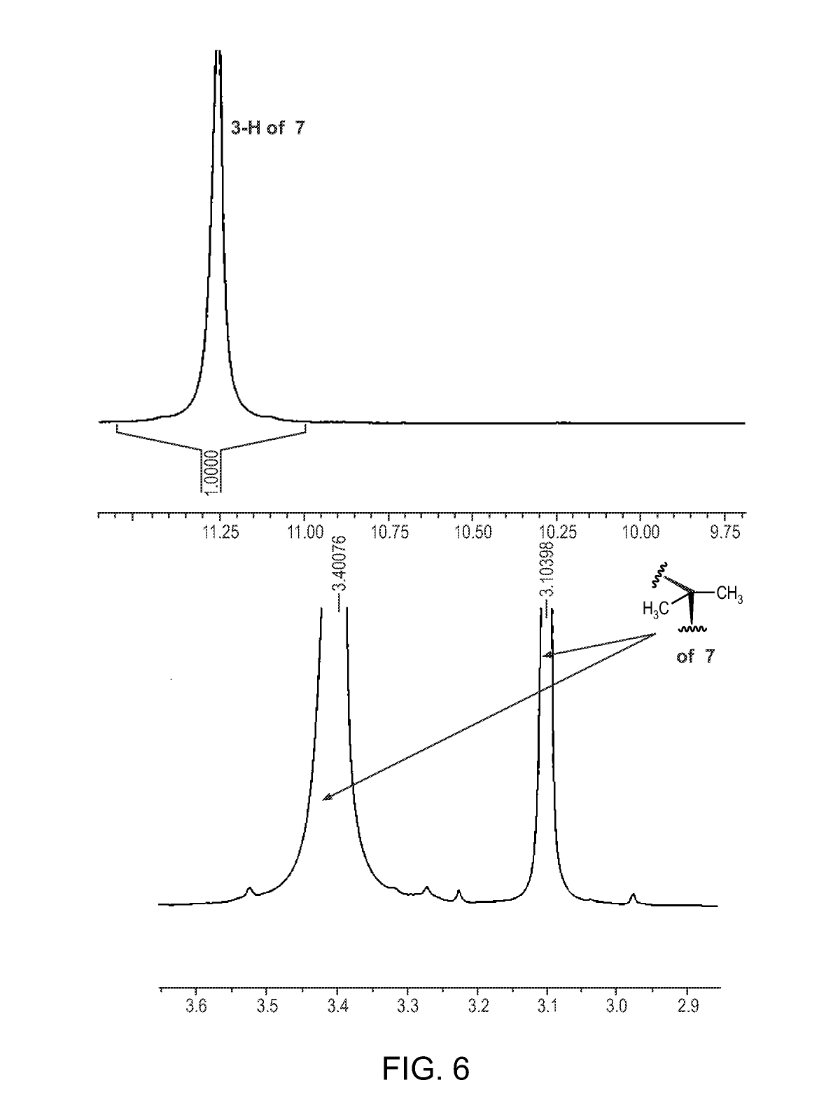

We also developed a second method for the direct determination of the enantiomeric purity of 7 and 10. This method involves ¹H NMR analysis using Eu(hfc)3 (Europium tris[3-heptafluoropropylhydroxymethylene)-(+)-camphorate]) as a chiral shift reagent. Solutions of 7, 10 and Eu(hfc)₃ were prepared in CDCl₃ in known ratios and their ¹H NMR spectra were recorded at 500 MHz. Among the ratios studied, 0.4:1 (mmol Eu(hfc)₃ : mmol 7+10) was found to be the best in the resolution of the enantiomeric protons (FIG. 4). With this method we found that 28 contains ˜4% of its enantiomer (FIG. 5) while 7 is enantiomerically pure (FIG. 6).

C. Synthesis of Compounds of the General Formula I, II and III.

Compounds 3.1-3.8, 3.11, 3.12, 3.14, 3.16, 4.1-4.6, 4.8-4.12, 4.14, 4.16, 5.3, 5.14, 6.10 and 6.11 were synthesized by a method depicted in Scheme 7.

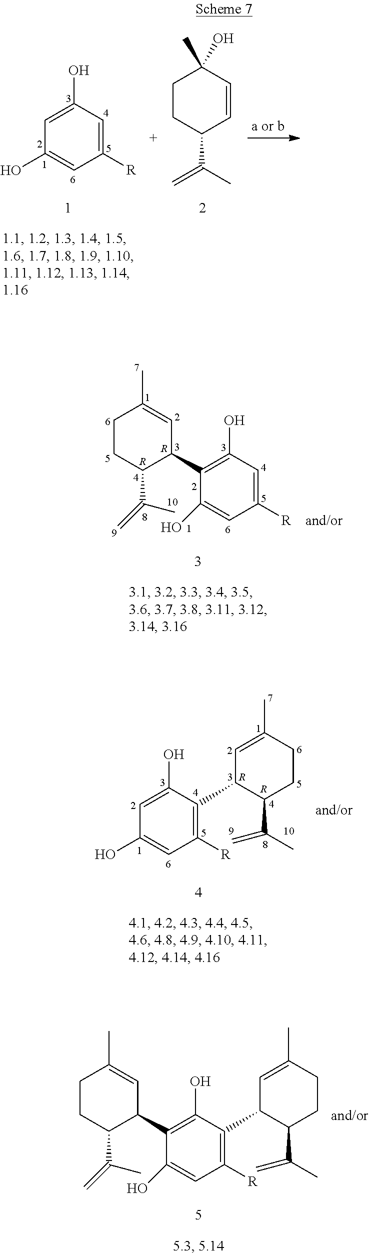

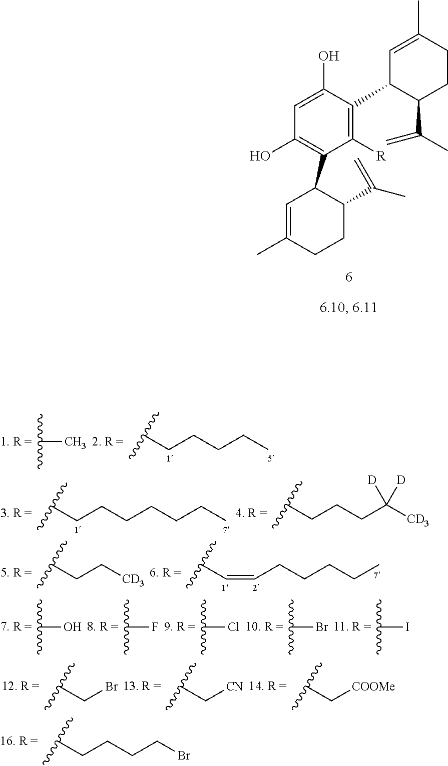

Reagents and conditions: (a) solvent (see experimental), p-TSA, 0° C. to r t; (b) benzene, molecular sieves 3 Å, Nafion-H, 5° C. to r t.

### General Experimental Procedure

To a stirred mixture of resorcinol 1 (1 equiv.) and p-TSA (approximately 0.01-0.1 equiv.) in an organic solvent (e.g. benzene, CH₂Cl₂, CH₂Cl₂/Et₂O approximately 0.03-0.5 M) at 0° C., under an argon atmosphere, was added a solution of (+)-cis/trans-p-mentha-2,8-dien-l-ol (2, approximately 1.1-1.5 equiv.) in the same organic solvent. Following the addition, the reaction temperature was raised to room temperature and stirring was continued for approximately 1-3 hours. The reaction was quenched by the addition of saturated sodium bicarbonate solution, the organic layer was separated and the aqueous phase was extracted with an organic solvent. The combined organic layer washed with brine, dried (MgSO₄) and the solvent was removed under reduced pressure. The residue was chromatographed on silica gel to afford the products 3 and/or 4 and/or 5 and/or 6.

### 1. (−)-2-[3-3,4-trans-p-Menthadien-(1,8)-yl]-5-methylresorcinol (Compound 3.1)

Yield: 11%; pale yellow viscous oil; ¹H NMR (500 MHz, CDCl₃) δ: 6.21 (br s, 2H, ArH), 5.91 (br s, 1H, OH), 5.55 (s, 1H, 2-H), 4.66 (s, 1H, 9-H), 4.62 (br s, 1H, OH), 4.57 (s, 1H, 9-H), 3.85 (m as br d, J=8.5 Hz, 1H, 3-H), 2.41 (ddd, J=10.0 Hz, J=8.5 Hz, J=2.2 Hz, 1H, 4-H), 2.28-2.17 (m, 4H, especially 2.19 s, Ar—CH₃), 2.14-2.05 (m, 1H), 1.89-1.72 (m and s overlapping, 5H, especially 1.79, s, 7-CH₃), 1.65 (s, 3H, 10-CH₃).

### 2. (−)-2-[3-3,4-trans-p-Menthadien-(1,8)-yl]-5-pentylresorcinol (Compound 3.2)

General procedure: Yield: 29%; viscous oil; ¹H NMR (500 MHz, CDCl₃) δ: 6.27 (br s, 1H, ArH), 6.17 (br s, 1H, ArH), 5.97 (br s, 1H, OH), 5.57 (s, 1H, 2-H), 4.68 (br s, 1H, OH), 4.66 (s, 1H, 9-H), 4.56 (s, 1H, 9-H), 3.85 (m as br d, J=10.3 Hz, 1H, 3-H), 2.44 (t, J=7.8 Hz, 2H, 140 -H), 2.40 (td, J=10.9 Hz, J=2.8 Hz, 1H, 4-H), 2.28-2.18 (m, 1H), 2.13-2.05 (m, 1H), 1.85-1.73 (m and s overlapping, 5H, especially 1.79 br s, 3H, 7-Me), 1.65 (s, 3H, 10-Me), 1.56 (quintet, J=7.4 Hz, 2H, 2′-H), 1.35-1.22 (m, 4H, 3′-H, 4′-H), 0.88 (t, J=6.9 Hz, 3H, 5′-H).

Alternative procedure: To a stirred mixture of olivetol (1 g, 5.55 mmol), (+)-cis/trans-p-mentha-2,8-dien-1-ol (1.35 g, 8.9 mmol) and molecular sieves 3A (0.57 g) in anhydrous benzene (11 ml) at 5° C., under an argon atmosphere, was added Nafion-H (beds, 30% w/w, 0.3 g). The reaction temperature was raised to room temperature and stirring was continued for 2 days. Solid materials were filtered off and the filtrate was evaporated under reduced pressure. The residue was chromatographed on silica gel (10-40% diethyl ether in hexane) to give 3.2 (528 mg, 30% yield) and 4.2 (682 mg, 39% yield).

### 3. (−)-2-[3-3,4-trans-p-Menthadien-(1,8)-yl]-5-heptylresorcinol (Compound 3.3)

Yield: 32%; colorless viscous oil. 1H NMR (500 MHz, CDCl₃) δ 6.26 (br s, 1H, ArH), 6.17 (br s, 1H, ArH), 5.97 (br s, 1H, OH), 5.57 (br s, 1H, 2-H), 4.66 (s, 1H, 9-H), 4.63 (br s, 1H, OH), 4.56 (s, 1H, 9-H), 3.84 (m as br d, J=8.7 Hz, 1H, 3-H), 2.44 (t, J=7.6 Hz, 2H, 1′-H), 2.39 (td, J=10.5 Hz, J=3.2 Hz, 1H, 4-H), 2.28-2.18 (m, 1H), 2.14-2.06 (m, 1H), 1.86-1.73 (m and s overlapping, 5H, especially 1.79, s, 3H, 7-CH₃), 1.65 (s, 3H, 10-CH₃), 1.58-1.51 (m, 2H, 2′-CH₂—), 1.34-1.23 (m, 8H, 3′-CH₂—, 4′-CH₂—, 5′-CH₂—, 6′-CH₂—), 0.87 (t, J=7.1 Hz, 3H, 7′-CH₃).

### 4. (—)-2-[3-3,4-trans-p-Menthadien-(1,8)-yl]-5-[4′-(²H₂)-5′-(²H₃)-pentyl]resorcinol (Compound 3.4)

Yield: 30%; viscous oil; ¹H NMR (500 MHz, CDCl₃) δ: 6.27 (br s, 1H, ArH), 6.17 (br s, 1H, ArH), 5.97 (br s, 1H, OH), 5.57 (s, 1H, 2-H), 4.66 (s, 1H, 9-H), 4.63 (br s, 1H, OH), 4.56 (s, 1H, 9-H), 3.85 (m as br d, 1H, J=9.1 Hz, 3-H), 2.44 (t, 2H, J=7.8 Hz, 1′-H), 2.40 (td, 1H, J=10.7 Hz, J=2.4 Hz, 4-H), 2.28-2.18 (m, 1H), 2.13-2.05 (m, 1H), 1.85-1.73 (m and s overlapping, 5H, especially 1.79 br s, 3H, 7-Me), 1.65 (s, 3H, 10-Me), 1.55 (quintet, 2H, J=7.7 Hz, 2′-H), 1.26 (t, 2H, J=7.5 Hz, 3′-H); mass spectrum m/z (relative intensity) 319 (M⁺, 16), 304 (8), 251 (17), 236 (100), 198 (13), 174 (23), 121 (17), 91 (26), 77 (20).

### 5. (−)-2-[3-3,4-trans-p-Menthadien-(1,8)-yl]-5-[3′-(²H₃)-propyl]resorcinol (Compound 3.5)

Yield: 32%; viscous oil; IR (neat) 3384, 2930, 2863, 2209 (w, C-D), 1627, 1586, 1518, 1436, 1311, 1240, 1147, 1050, 1024, 1004, 891, 844 cm⁻¹. ¹H NMR (500 MHz, CDCl₃) δ: 6.28 (br s, 1H, ArH), 6.17 (br s, 1H, ArH), 5.95 (br s, 1H, OH), 5.57 (s, 1H, 2-H), 4.66 (s, 1H, 9-H), 4.61 (br s, 1H, OH), 4.56 (s, 1H, 9-H), 3.84 (m as br d, J=9.3 Hz, 1H, 3-H), 2.42 (t, J=7.7 Hz, 2H, 1′-H), 2.39 (td, J=10.7 Hz, J=2.7 Hz, 1H, 4-H), 2.29-2.19 (m, 1H), 2.14-2.06 (m, 1H), 1.86-1.74 (m and s, overlapping, 5H, especially 1.79, s, 7-Me), 1.65 (s, 3H, 10-Me), 1.56 (m, 2H, 2′-H); mass spectrum m/z (relative intensity) 289 (M⁺, 23), 274 (11), 221 (24), 206 (100), 174 (31), 168 (17), 121 (14), 91 (28), 77 (27).

### 6. (−)-2-[3-3,4-trans-p-Menthadien-(1,8)-yl]-5-(1,2-cis-hepten-1-yl)resorcinol (Compound 3.6)

Yield: 31%; viscous oil; ¹H NMR (500 MHz, CDCl₃) δ: 6.41 (br s, 1H, ArH), 6.27 (br s, 1H, ArH), 6.21 (d, J=11.7 Hz, 1H, 1′-H), 6.00 (br s, 1H, OH), 5.59 (dt, J=11.7, J=7.2 Hz, 1H, 2′-H), 5.57 (s, 1H, 2-H), 4.69 (br s, 1H, OH), 4.67 (s, 1H, 9-H), 4.57 (s, 1H, 9-H), 3.88 (m as br d, J=8.7 Hz, 1H, 3-H), 2.42 (td, J=11.4 Hz, J=3.1 Hz, 1H, 4-H), 2.32 (qd, J=7.2 Hz, J=1.5 Hz, 2H, 3′-CH₂—), 2.27-2.20 (m, 1H), 2.15-2.07 (m, 1H), 1.89-1.75 (m and s overlapping, 5H, especially 1.80, s, 7-CH₃), 1.67 (s, 3H, 10-CH₃), 1.43 (quintet, J=7.0 Hz, 2H, 4′-CH₂—), 1.35-1.22 (m, 4H, 5′-CH₂—, 6′-CH₂—), 0.88 (t, J=6.9 Hz, 3H, 7′-CH₃); mass spectrum m/z (relative intensity) 340 (M⁺, 27), 272 (55), 257 (100), 219 (15), 187 (6), 149 (4), 121 (16), 77 (6). Exact mass calculated for C₂₃H₃₂O₂, 340.2402; found, 340.2406.

### 7. (−)-2-[3-3,4-trans-p-Menthadien-(1,8)-yl]-5-hydroxyresorcinol (Compound 3.7)

¹H NMR (500 MHz, CDCl₃) δ: 6.10 (br s, 1H, OH), 5.95 (br s, 1H, ArH), 5.88 (br s, 1H, ArH), 5.54 (s, 1H, 2-H), 4.92 (br s, 2H, OH), 4.67 (s, 1H, 9-H), 4.57 (s, 1H, 9-H), 3.77 (m as br d, J=8.9 Hz, 1H, 3-H), 2.34 (td, J=11.7 Hz, J=3.3 Hz, 1H, 4-H), 2.28-2.18 (m, 1H), 2.14-2.06 (m, 1H), 1.85-1.72 (m and s overlapping, 5H, especially 1.79, s, 7-CH₃), 1.66 (s, 3H, 10-CH₃); mass spectrum (FAB) m/z (relative intensity) 261 (M⁺+1, 100), 192 (55), 177 (38), 155 (22), 135 (51), 119 (61). Exact mass calculated for C₁₆H₂₁O₃ (M⁺+1): 261.1491; found, 261.1492.

### 8. (−)-2-[3-3,4-trans-p-Menthadien-(1,8)-yl]-5-flouororesorcinol (Compound 3.8)

Yield: 12%; yellow gum. IR (neat) 3410, 2926, 1608, 1510, 1448, 1222, 1120, 1030, 1001, 890, 823 cm⁻¹. ¹H NMR (500 MHz, CDCl₃) δ: 6.24 (br s, 1H, OH), 6.18 (br s, 1H, ArH), 6.06 (br s, 1H, ArH), 5.54 (s, 1H, 2-H), 5.24 (br s, 1H, OH), 4.65 (s, 1H, 9-H), 4.53 (s, 1H, 9-H), 3.83 (m as br d, J=9.8 Hz, 1H, 3-H), 2.36 (td, J=11.2 Hz, J=3.2 Hz, 1H, 4-H), 2.28-2.18 (m, 1H), 2.14-2.06 (m, 1H), 1.89-1.72 (m and s overlapping, 5H, especially 1.80, s, 7-CH₃), 1.65 (s, 3H, 10-CH₃); mass spectrum m/z (relative intensity) 262 (M⁺, 14), 247(2), 194(45), 179(100), 141(7), 121(13), 108(8).

### 9. (−)-2-[3-3,4-trans-p-Menthadien-(1,8)-yl]-5-iodoresorcinol (Compound 3.11)

Yield: 9%; white solid; m p=128-130° C.; IR (neat) 3437, 3306, 2934, 1643, 1599, 1574, 1488, 1426, 1305, 1221, 1026, 900, 808 cm⁻¹; ¹H NMR (500 MHz, CDCl₃) δ: 6.82 (br s, 1H, ArH), 6.68 (br s, 1H, ArH), 6.09 (br s, 1H, OH), 5.51 (s, 1H, 2-H), 4.87 (br s, 1H, OH), 4.65 (s, 1H, 9-H), 4.53 (s, 1H, 9-H), 3.85 (m as br d, J=8.7 Hz, 1H, 3-H), 2.37 (td, J=11.7 Hz, J=3.2 Hz, 1H, 4-H), 2.28-2.19 (m, 1H), 2.12-2.07 (m, 1H), 1.85-1.72 (m and s overlapping, 5H, especially 1.80, s, 7-CH₃), 1.66 (s, 3H, 10-CH₃).

### 10. (−)-2-[3-3,4-trans-p-Menthadien-(1,8)-yl]-5-(bromomethyl)resorcinol (Compound 3.12)

Yield: 24%; ¹H NMR (500 MHz, CDCl₃) δ: 6.48 (br s, 1H, ArH), 6.38 (br s, 1H, ArH), 6.10 (br s, 1H, OH), 5.54 (s, 1H, 2-H), 4.87 (br s, 1H, OH), 4.65 (s, 1H, 9-H), 4.53 (s, 1H, 9-H), 4.35 (s, 2H, —CH₂Br), 3.89 (m as br d, J=8.5 Hz, 1H, 3-H), 2.39 (td, J=11.0 Hz, J=3.2 Hz, 1H, 4-H), 2.29-2.19 (m, 1H), 2.15-2.07 (m, 1H), 1.87-1.73 (m and s overlapping, 5H, especially 1.80, s, 7-CH₃), 1.66 (s, 3H, 10-CH₃).

### 11. (−)-2-[3-3,4-trans-p-Menthadien-(1,8)-yl]-5-(2-methoxy-2-oxoethyl)resorcinol (Compound 3.14)

Yield: 28%; white gum; IR (neat) 3428, 2925, 1717 (s, >C═O), 1625, 1587, 1435, 1376, 1303, 1240, 1148, 1053, 1015, 1032, 887, 833 cm⁻¹; ¹H NMR (500 MHz, CDCl₃) δ: 6.33 (br s, 1H, ArH), 6.29 (br s, 1H, ArH), 6.04 (br s, 1H, OH), 5.55 (s, 1H, 2-H), 4.99 (br s, 1H, OH), 4.63 (s, 1H, 9-H), 4.53 (s, 1H, 9-H), 3.89 (m as br d, J=8.7 Hz, 1H, 3-H), 3.69 (s, 3H, —CH₂COOCH₃), 3.46 (s, 2H, —CH₂COOCH₃), 2.40 (td, J=11.2 Hz, J=3.3 Hz, 1H, 4-H), 2.29-2.19 (m, 1H), 2.14-2.05 (m, 1H), 1.86-1.74 (m and s overlapping, 5H, especially 1.79, s, 7-Me), 1.66 (s, 3H, 10-Me); mass spectrum (FAB) m/z (relative intensity) 317 (M⁺+1, 25), 233 (26), 154 (94), 136 (100). Exact mass calculated for C₁₉H₂₅O₄ (M⁺+1) 317.1753; found, 317.1749.

### 12. (−)-2-[3-3,4-trans-p-Menthadien-(1,8)-yl]-5-(4-bromobutyl)resorcinol (Compound 3.16)

Yield: 31%; yellow viscous oil; ¹H NMR (500 MHz, CDCl₃) δ: 6.27 (br s, 1H, ArH), 6.16 (br s, 1H, ArH), 6.00 (br s, 1H, OH), 5.56 (s, 1H, 2-H), 4.68 (br s, 1H, OH), 4.66 (s, 1H, 9-H), 4.55 (s, 1H, 9-H), 3.84 (m as br d, J=8.7 Hz, 1H, 3-H), 3.40 (t, J=6.7 Hz, 2H, 4′-H), 2.48 (t, J=7.5 Hz, 2H, 1′-H), 2.39 (td, J=11.0 Hz, J=3.1 Hz, 1H, 4-H), 2.27-2.20 (m, 1H), 2.13-2.07 (m,1H), 1.88-1.76 (m, 7H, especially 1.80, s, 7-CH₃), 1.75-1.69 (m, 2H), 1.66 (s, 3H, 10-CH₃); mass spectrum m/z (relative intensity) 380 (M⁺+2, 15), 378 (M⁺, 15), 312(33), 310(34), 299(100), 297(100), 215(31), 121(27). Exact mass calculated for C₂₀H₂₇BrO₂, 378.1194; found, 378.1192. Anal. Calcd: C, 63.33; H, 7.17. Found: C, 63.15; H, 6.88.

### 13. (−)-4-[3-3,4-trans-p-Menthadien-(1,8)-yl]-5-methylresorcinol (Compound 4.1)

Yield: 25%; yellow gum; ¹H NMR (200 MHz, CDCl₃) δ: 6.22 (d, J=2.5 Hz, 1H, ArH), 6.19 (d, J=2.5 Hz, 1H, ArH), 6.14 (s, 1H, OH), 5.54 (s, 1H, 2-H), 5.28 (s, 1H, OH), 4.65 (s, 1H, 9-H), 4.46 (s, 1H, 9-H), 3.55 (m as br d, J=9.1 Hz, 1H, 3-H), 2.44 (td, J=10.2 Hz, J=4.1 Hz, 1H, 4-H), 2.30-1.98 (m and s overlapping, 5H, especially 2.14, s, Ar—CH₃), 1.90-1.65 (m and s overlapping, 5H, especially 1.78, s, 7-Me), 1.56 (s, 3H, 10-Me).

### 14. (−)-4-[3-3,4-trans-p-Menthadien-(1,8)-yl]-5-pentylresorcinol (Compound 4.2)

Yield: 40%; pale yellow gum; ¹H NMR (500 MHz, CDCl₃) δ: 6.20 (d, J=2.6 Hz, 1H, ArH), 6.19 (d, J=2.6 Hz, 1H, ArH), 6.05 (s, 1H, OH), 5.52 (s, 1H, 2-H), 4.64 (s, 1H, 9-H), 4.62 (s, 1H, OH), 4.46 (s, 1H, 9-H), 3.53 (m as br d, J=8.3 Hz, 1H, 3-H), 2.59 (dt, J=14.3 Hz, J=7.2 Hz, 1H, 1′-H), 2.47 (td, J=11.1 Hz, J=3.1 Hz, 1H, 4-H), 2.30-2.17 (m and dt overlapping, 2H, especially 2.25, dt, 1′-H), 2.14-2.05 (m, 1H), 1.87-1.72 (m and s overlapping, 5H, especially 1.79, s, 7-Me), 1.53 (s, 3H, 10-Me), 1.52-1.42 (m, 2H, 2′-H), 1.37-1.24 (m, 4H, 3′-H, 4′-H), 0.89 (t, J=7.1 Hz, 3H, 5′-H)

Following the alternative procedure (see 3.2 above) the title compound was isolated in 39% yield.

### 15. (−)-4-[3-3,4-trans-p-Menthadien-(1,8)-yl]-5-heptylresorcinol (Compound 4.3)

Yield: 41%; pale yellow gum; ¹H NMR (500 MHz, CDCl₃) δ: 6.20 (d, J=2.5 Hz, 1H, ArH), 6.19 (d, J=2.5 Hz, 1H, ArH), 6.03 (s, 1H, OH), 5.52 (s, 1H, 2-H), 4.64 (s, 1H, 9-H), 4.49 (s, 1H, OH), 4.46 (s, 1H, 9-H), 3.52 (m as br d, J=9.2 Hz, 1H, 3-H), 2.59 (dt, J=14.5 Hz, J=7.1 Hz, 1H, 1′-H), 2.47 (td, J=11.1 Hz, J=2.9 Hz, 1H, 4-H), 2.29-2.16 (m and dt overlapping, 2H, especially 2.25, dt, 1′-H), 2.14-2.06 (m, 1H), 1.88-1.72 (m and s overlapping, 5H, especially 1.79, s, 7-Me), 1.55 (s, 3H, 10-Me), 1.50-1.43 (m, 2H, 2′-H), 1.34-1.23 (m, 8H, 3′-H, 4′-H, 5′-H, 6′-H), 0.88 (t, J=7.1 Hz, 3H, 7′-H).

### 16. (−)-4-[3-3,4-trans-p-Menthadien-(1,8)-yl]-5-[4′-(²H₂)-5′-(²H₃)-pentyl]resorcinol (Compound 4.4)

Yield: 39%; pale yellow gum; ¹H NMR (500 MHz, CDCl₃) δ: 6.20 (d, J=2.5 Hz, 1H, ArH), 6.19 (d, J=2.5 Hz, 1H, ArH), 6.04 (s, 1H, OH), 5.52 (s, 1H, 2-H), 4.64 (s, 1H, 9-H), 4.53 (s, 1H, OH), 4.46 (s, 1H, 9-H), 3.52 (m as br d, J=8.3 Hz, 1H, 3-H), 2.59 (dt, J=14.3 Hz, J=7.2 Hz, 1H, 1′-H), 2.47 (td, J=11.1 Hz, J=3.1 Hz, 1H, 4-H), 2.30-2.18 (m and dt overlapping, 2H, especially 2.25, dt, 1′-H), 2.14-2.05 (m, 1H), 1.87-1.72 (m and s overlapping, 5H, especially 1.79, s, 7-Me), 1.53 (s, 3H, 10-Me), 1.51-1.41 (m, 2H, 2′-H), 1.28 (t, J=7.6 Hz, 2H, 3′-H).

### 17. (−)-4-[3-3,4-trans-p-Menthadien-(1,8)-yl]-5-[3′-(²H₃)-propyl]resorcinol (Compound 4.5)

Yield: 47%; pale yellow gum; IR (neat) 3417, 2927, 2873, 2214 (w, C-D), 1619, 1592, 1446, 1148, 1133, 1062, 995, 887, 843 cm⁻¹; ¹H NMR (500 MHz, CDCl₃) δ: 6.21 (d, J=2.6 Hz, 1H, ArH), 6.19 (d, J=2.6 Hz, 1H, ArH), 6.04 (s, 1H, OH), 5.52 (s, 1H, 2-H), 4.65 (s, 1H, 9-H), 4.61 (s, 1H, OH), 4.46 (s, 1H, 9-H), 3.53 (m as br d, J=8.3 Hz, 1H, 3-H), 2.58 (m, 1H, 1′-H), 2.48 (td, J=11.1 Hz, J=3.1 Hz, 1H, 4-H), 2.28-2.18 (m, 2H), 2.14-2.05 (m, 1H), 1.87-1.72 (m and s overlapping, 5H, especially 1.79, s, 7-Me), 1.53 (s, 3H, 10-Me), 1.51-1.41 (m, 2H, 2′-H).

### 18. (−)-4-[3-3,4-trans-p-Menthadien-(1,8)-yl]-5-(1,2-cis-hepten-1-yl)resorcinol (Compound 4.6)

Yield: 43%; pale yellow gum; ¹H NMR (500 MHz, CDCl₃) δ: 6.28 (d, J=11.3 Hz, 1H, 1′-H), 6.26 (d, J=2.0 Hz, 1H, ArH), 6.14 (d, J=2.0 Hz, 1H, ArH), 6.01 (s, 1H, OH), 5.60 (dt, J=11.3, J=7.3 Hz, 1H, 2′-H), 5.53 (s, 1H, 2-H), 4.62 (s, 1H, 9-H), 4.56 (s, 1H, OH), 4.38 (s, 1H, 9-H), 3.57 (m as br d, J=8.6 Hz, 1H, 3-H), 2.46 (td, J=10.1 Hz, J=2.6 Hz, 1H, 4-H), 2.28-2.18 (m, 1H), 2.12-1.92 (m, 3H), 1.90-1.64 (m and s overlapping, 5H, especially 1.78, s, 7-CH₃), 1.57 (s, 3H, 10-CH₃), 1.36 (quintet, J=7.1 Hz, 2H, 4′-CH₂—), 1.30-1.23 (m, 4H, 5′-CH₂—, 6′-CH₂—), 0.87 (t, J=6.9 Hz, 3H, 7-CH₃); mass spectrum m/z (relative intensity) 340 (M⁻, 44), 272 (73), 257 (33), 231 (45), 215 (27), 201 (100), 187 (34), 174 (72), 110 (21), 82 (33). Exact mass calculated for C₂₃H₃₂O₂, 340.2402; found, 340.2398.

### 19. (−)-4-[3-3,4-trans-p-Menthadien-(1,8)-yl]-5-flouororesorcinol (Compound 4.8)

Yield: 27%; white solid; m p=105-106° C.; IR (neat) 3315, 2926, 1632, 1516, 1467, 1334, 1259, 1145, 1132, 1024, 1003, 879, 830 cm⁻¹; ¹H NMR (500 MHz, CDCl₃) δ: 6.17-6.06 (m, 3H, ArH and OH overlapping), 5.53 (s, 1H, 2-H), 5.07 (s, 1H, OH), 4.59 (s, 1H, 9-H), 4.41 (s, 1H, 9-H), 3.74 (m as br d, J=9.2 Hz, 1H, 3-H), 2.36 (td, J =4.1 Hz, 1H, 4-H), 2.28-2.18 (m, 1H), 2.14-2.06 (m, 1H), 1.85-1.71 (m and s overlapping, 5H, especially 1.79, s, 7-Me), 1.65 (s, 3H, 10-Me); Mass spectrum m/z (relative intensity) 262 (M⁺, 15), 247(3), 194(81), 179(100), 141(9), 121(14), 108(12). Exact mass calculated for C₁₆H₁₉FO₂, 262.1369; found, 262.1370.

### 20. (−)-4-[3-3,4-trans-p-Menthadien-(1,8)-yl]-5-chlororesorcinol (Compound 4.9)

Yield: 39%; white solid; m p=101-103° C.; IR (neat) 3339, 2934, 1644, 1608, 1498, 1447, 1374, 1309, 1147, 1123, 1000, 870, 840 cm⁻¹; ¹H NMR (500 MHz, CDCl₃) δ: 6.42 (d, J=2.2 Hz, 1H, ArH), 6.26 (d, J=2.2 Hz, 1H, ArH), 6.24 (s, 1H, OH), 5.54 (s, 1H, 2-H), 4.78 (s, 1H, OH), 4.60 (s, 1H, 9-H), 4.37 (s, 1H, 9-H), 3.97 (m as br d, J=9.0 Hz, 1H, 3-H), 2.47 (td, J=10.4 Hz, J=4.2 Hz, 1H, 4-H), 2.27-2.17 (m, 1H), 2.12-2.08 (m, 1H), 1.84-1.74 (m and s overlapping, 5H, especially 1.80, s, 7-Me), 1.68 (s, 3H, 10-Me); mass spectrum m/z (relative intensity) 280 (M⁺2, 5), 278 (M⁺, 15), 263 (2), 243 (4), 212 (20), 210 (60), 197 (22), 195 (68), 175 (100), 160 (8), 157 (11), 121 (24). Exact mass calculated for C₁₆H₁₉ClO₂, 278.1074; found, 278.1075.

### 21. (−)-4-[3-3,4-trans-p-Menthadien-(1,8)-yl]-5-bromoresorcinol (Compound 4.10)

Yield: 46%; white solid; m p=91-93° C.; IR (neat) 3341, 2927, 1603, 1494, 1444, 1373, 1306, 1148, 1121, 997, 880, 825 cm⁻¹; ¹H NMR (500 MHz, CDCl₃) δ: 6.62 (d, J=1.9 Hz, 1H, ArH), 6.31 (d, J=1.9 Hz, 1H, ArH), 6.27 (s, 1H, OH), 5.55 (s, 1H, 2-H), 4.91 (br s, 1H, OH), 4.62 (s, 1H, 9-H), 4.37 (s, 1H, 9-H), 3.96 (m as br d, J=9.0 Hz, 1H, 3-H), 2.49 (td, J=10.4 Hz, J=3.7 Hz, 1H, 4-H), 2.26-2.17 (m, 1H), 2.12-2.07 (m, 1H), 1.82-1.75 (m and s overlapping, 5H, especially 1.79, s, 7-Me), 1.70 (s, 3H, 10-Me); mass spectrum m/z (relative intensity) 324 (M⁺+2, 8), 322 (M⁺, 8), 256 (21), 254 (21), 241 (22), 239 (22), 175 (100), 160 (14), 121 (19). Exact mass calculated for C₁₆H₁₉BrO₂, 322.0568; found, 322.0571.

### 22. (−)-4-[3-3,4-trans-p-Menthadien-(1,8)-yl]-5-iodoresorcinol (Compound 4.11)

Yield: 52%; yellow gum; IR (neat) 3395, 2924, 1643, 1610, 1577, 1481, 1432, 1375, 1279, 1169, 1146, 1119, 1048, 998, 890, 839, 805 737 cm⁻¹; ¹H NMR (500 MHz, CDCl₃) δ: 6.91 (d, J=2.3 Hz, 1H, ArH), 6.34 (d, J=2.3 Hz, 1H, ArH), 6.25 (s, 1H, OH), 5.55 (s, 1H, 2-H), 4.75 (s, 1H, OH), 4.65 (s, 1H, 9-H), 4.39 (s, 1H, 9-H), 3.80 (m as br d, J=8.9 Hz, 1H, 3-H), 2.51 (td, J=10.3 Hz, J=4.2 Hz, 1H, 4-H), 2.27-2.16 (m, 1H), 2.11-2.08 (m, 1H), 1.85-1.75 (m and s overlapping, 5H, especially 1.80, s, 7-Me), 1.71 (s, 3H, 10-Me); mass spectrum m/z (relative intensity) 370 (M⁺, 20), 355 (2), 302 (27), 287 (18), 264 (11), 249 (7), 243 (5), 175 (100), 160 (22), 121 (25). Exact mass calculated for C₁₆H₁₉IO₂, 370.0430; found, 370.0431.

### 23. (−)-4-[3-3,4-trans-p-Menthadien-(1,8)-yl]-5-(bromomethyl)resorcinol (Compound 4.12)

Yield: 26%; ¹H NMR (500 MHz, CDCl₃) δ: 6.35 (d, J=2.5 Hz, 1H, ArH), 6.33 (d, J=2.5 Hz, 1H, ArH), 6.18 (s, 1H, OH), 5.58 (s, 1H, 2-H), 4.77 (s, 1H, OH), 4.70 (s, 1H, 9-H), 4.51 (s, 1H, 9-H), 4.48 (d, J=10.3 Hz, 1H, —CH₂Br), 4.16 (d, J=10.3 Hz, 1H, CH₂Br), 3.66 (m as br d, J=9.1 Hz, 1H, 3-H), 2.46 (td, J=11.8 Hz, J=3.4 Hz, 1H, 4-H), 2.29-2.19 (m, 1H), 2.17-2.07 (m, 1H), 1.90-1.71 (m and s overlapping, 5H, especially 1.81, s, 7-Me), 1.53 (s, 3H, 10-Me).

### 24. (−)-4-[3-3,4-trans-p-Menthadien-(1,8)-yl]-5-(2-methoxy-2-oxoethyl)resorcinol (Compound 4.14)

Yield: 19%; white foam; IR (neat) 3419, 2926, 1709 (s, >C═O), 1615, 1595, 1436, 1375, 1242, 1149, 1135, 1049, 1015, 889, 843 cm⁻¹; ¹H NMR (500 MHz, CDCl₃) δ: 6.30 (d, J=2.5 Hz, 1H, ArH), 6.22 (d, J=2.5 Hz, 1H, ArH), 6.14 (s, 1H, OH), 5.54 (s, 1H, 2-H), 4.90 (br s, 1H, OH), 4.67 (s, 1H, 9-H), 4.49 (s, 1H, 9-H), 3.71 (d, J=16.0 Hz, 1H, —CH₂COOCH₃), 3.68 (s, 3H, —CH₂COOCH₃), 3.48 (m as br d, J=9.9 Hz, 1H, 3-H), 3.31 (d, J=16.0 Hz, 1H, —CH₂COOCH₃), 2.44 (td, J=10.7 Hz, J=3.1 Hz, 1H, 4-H), 2.28-2.17 (m, 1H), 2.15-2.05 (m, 1H), 1.86-1.72 (m and s overlapping, 5H, especially 1.79, s, 7-Me), 1.52 (s, 3H, 10-Me); mass spectrum (FAB) m/z (relative intensity) 317 (M⁺+1, 32), 233 (38), 154 (100), 136 (95). Exact mass calculated for C₁₉H₂₅O₄ (M⁺+1) 317.1753; found, 317.1749.

### 25. (−)-4-[3-3,4-trans-p-Menthadien-(1,8)-yl1-5-(4-bromobutyl)resorcinol (Compound 4.16)

Yield: 36%; yellow viscous oil; ¹H NMR (500 MHz, CDCl₃) δ: 6.22 (d, J=2.6 Hz, 1H, ArH), 6.19 (d, J=2.6 Hz, 1H, ArH), 6.08 (s, 1H, OH), 5.52 (s, 1H, 2-H), 4.77 (br s, 1H, OH), 4.65 (s, 1H, 9-H), 4.47 (s, 1H, 9-H), 3.52 (m as br d, J=8.3 Hz, 1H, 3-H), 3.41 (t, J=6.8 Hz, 2H, 4′-H), 2.68-2.62 (m, 1H), 2.49-2.44 (m, 1H), 2.31-2.19 (m, 2H), 2.12-2.05 (m, 1H), 2.89-2.73 (m, 7H, especially 1.79, s, 7-CH₃), 1.67-1.57 (m, 2H), 1.52 (s, 3H, 10-CH₃); mass spectrum m/z (relative intensity) 380 (M⁺+2, 33), 378 (M⁺, 33), 312(78), 310(78), 297(83), 295(83), 269 (17), 215 (29), 189 (64), 175 (100), 147 (20), 91 (25). Exact mass calculated for C₂₀H₂₇BrO₂, 378.1194; found, 378.1193.

### 26. 2,4-bis-[3-3,4-trans-p-Menthadien-(1,8)-yl]-5-heptylresorcinol (Compound 5.3)

Yield: 18%; pale yellow gum; ¹H NMR (500 MHz, CDCl₃) δ: 6.20 (s, 1H, ArH), 5.91 (s, 1H, OH), 5.77 (br s, 1H, OH), 5.58 (s, 1H, —(CH₃)C═C(H)—), 5.49 (s, 1H, —(CH₃)C═C(H)—), 4.60 (s, 1H, —(CH₃)C═CH₂), 4.50 (s, 1H, —(CH₃)C═CH₂), 4.45 (s, 1H, —(CH₃)C═CH₂), 4.43 (s, 1H, —(CH₃)C═CH₂), 4.00 (m as br d, J=8.7 Hz, 1H, ═C—CH<), 3.48 (m as br d, J=8.7 Hz, 1H, ═CH—CH<), 2.53 (dt, J=14.4 Hz, J=7.0 Hz, 1H, 1′-H), 2.50-2.41 (m, 2H, 2 x >CH—C(CH₃)═CH₂), 2.29-2.15 (m, 3H), 2.13-2.04 (m, 2H), 1.87-1.72 (m, s and s overlapping, 10H, especially 1.78, s and 1.76 s, 2 x —(CH₃)C═C(H)—), 1.70 (s, 3H, —(CH₃)C═CH₂), 1.49 (s, 3H, —(CH₃)C═CH₂), 1.48-1.42 (m, 2H, 2′-H), 1.34-1.22 (m, 8H, 3′-H, 4′-H, 5′-H, 6′-H), 0.88 (t, J=7.0 Hz, 3H, 7′-H).

### 27. 2,4-bis-[3-3,4-trans-p-Menthadien-(1,8)-yl]-5-(2-methoxy-2-oxoethyl)resorcinol (Compound 5.14)

Yield: 19%; gum; IR (neat) 3425, 2925, 1737 (s, >C═O) , 1644, 1616, 1583, 1432, 1376, 1257, 1151, 1013, 886 cm⁻¹; ¹H NMR (500 MHz, CDCl₃) δ: 6.22 (s, 1H, ArH), 6.03 (s, 1H, OH), 5.83 (s, 1H, OH), 5.57 (s, 1H, —(CH₃)C═C(H)—), 5.51 (s, 1H, —(CH₃)C═C(H)—), 4.63 (s, 1H, —(CH₃)C═CH₂), 4.49 (s, 1H, —(CH₃)C═CH₂), 4.47 (s, 1H, —(CH₃)C═CH₂), 4.43 (s, 1H, —(CH₃)C═CH₂), 4.02 (m as br d, J=8.9 Hz, 1H, ═CH—CH<), 3.67 (d, J=16.0 Hz, 1H, —CH₂COOCH₃), 3.66 (s, 3H, —CH₂COOCH₃), 3.44 (m as br d, J=8.9 Hz, 1H, ═CH—CH<), 2.26 (d, J=16.0 Hz, 1H, —CH₂COOCH₃), 2.50-2.35 (m, 2H, 2 x >CH—C(CH₃)═CH₂), 2.28-2.17 (m, 2H), 2.12-2.03 (m, 2H), 1.84-1.72 (m, s and s overlapping, 10H, especially 1.78, s and 1.77 s, 2 x —(CH₃)C═C(H)—), 1.70 (s, 3H, —(CH₃)C═CH₂), 1.49 (s, 3H, —(CH₃)C═CH₂).

### 28. 2,6-bis-[3-3,4-trans-p-Menthadien-(1,8)-yl]-5-bromoresorcinol (Compound 6.10)

Yield: 20%; gum; 1H NMR (500 MHz, CDCl₃) δ: 6.33 (s, 1H, ArH), 6.24 (s, 2H, OH), 5.57 (s, 2H, 2-H), 4.60 (s, 2H, 9-H), 4.37 (s, 2H, 9-H), 4.12 (m as br d, J=7.9 Hz, 2H, 3-H), 2.52 (td, J=10.0 Hz, J=3.8 Hz, 2H, 4-H), 2.25-2.15 (m, 2H), 2.13-2.03 (m, 2H), 1.85-1.75 (m and s overlapping, 10H, especially 1.78, s, 6H, 7-Me), 1.66 (s, 6H, 10-Me).

### 29. 2,6-bis-[3-3,4-trans-p-Menthadien-(1,8)-yl]-5-iodoresorcinol (Compound 6.11)

Yield: 19%; gum; 1H NMR (500 MHz, CDCl₃) δ: 6.36 (s, 1H, ArH), 6.26 (s, 2H, OH), 5.57 (s, 2H, 2-H), 4.63 (m as t, J=1.5 Hz, 2H, 9-H), 4.38 (m, J=0.7 Hz, 2H, 9-H), 4.11 (m as br d, J=8.0 Hz, 2H, 3-H), 2.56 (td, J=10.0 Hz, J=4.4 Hz, 2H, 4-H), 2.22-2.12 (m, 2H), 2.11-2.02 (m, 2H), 1.84-1.75 (m and s overlapping, 10H, especially 1.77, s, 6H, 7-Me), 1.67 (s, 6H, 10-Me).

Compounds 3.13, 3.15, 4.13 and 4.15 were synthesized by a method depicted in Scheme 8.

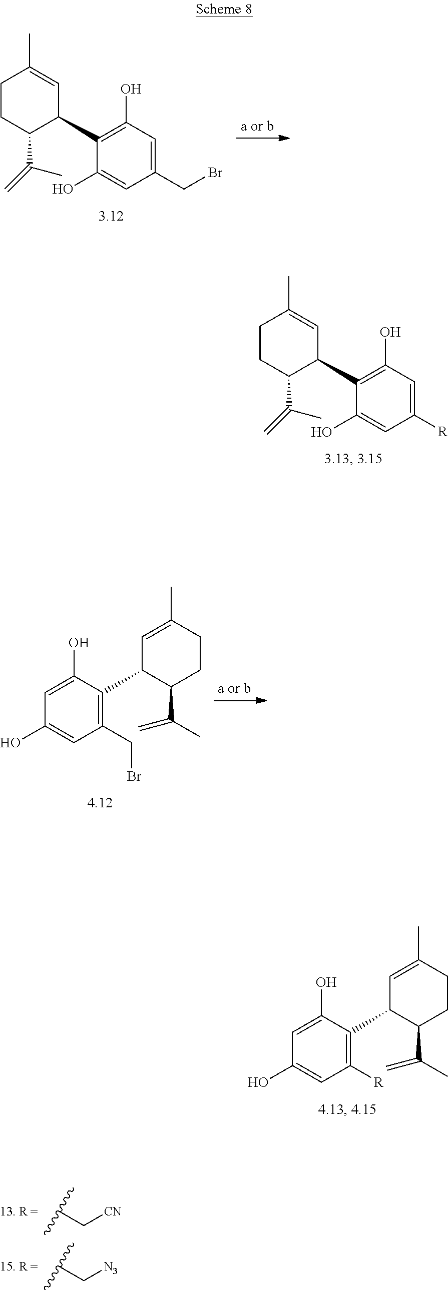

Reagents and conditions: (a) NaCN, DMSO, r t; (b) TMG-N₃, CH₂Cl₂/CH₃NO₂, reflux.

### General Experimental Procedure:

### 1. (−)-2-[3-3,4-trans-p-Menthadien-(1,8)-yl]-5-(cyanomethyl)resorcinol (Compound 3.13)

To a stirred solution of 3.12 (57 mg, 0.15 mmol) in DMSO (3 ml), at room temperature, under an argon atmosphere, was added NaCN (37 mg, 0.75 mmol). After stirring at the same temperature for 20 h, the reaction mixture was cooled to 0° C. and diluted with water. The mixture was extracted with diethyl ether and the organic layer was washed with brine, dried over MgSO₄ and the solvent was removed in vacuo. The residue was purified by flash column chromatography on silica gel (EtOAc in hexane) to give 3.13 in 54% yield (23 mg). IR (neat) 3346, 3277, 2927, 2262 (w, —C≡N), 1682, 1619, 1589, 1443, 1376, 1246, 1150, 1054, 1031, 891, 828 cm⁻¹; ¹H NMR (500 MHz, CDCl₃) δ: 6.36 (br s, 2H, ArH), 6.14 (br s, 1H, OH), 5.53 (s, 1H, 2-H), 5.14 (br s, 1H, OH), 4.63 (s, 1H, 9-H), 4.51 (s, 1H, 9-H), 3.90 (m as br d, J=8.4 Hz, 1H, 3-H), 3.61 (s, 2H, —CH₂CN), 2.39 (td, J=11.0 Hz, J=3.5 Hz, 1H, 4-H), 2.30-2.20 (m, 1H), 2.14-2.06 (m, 1H), 1.87-1.72 (m and s overlapping, 5H, especially 1.80, s, 7-CH₃), 1.67 (s, 3H, 10-CH₃); mass spectrum m/z (relative intensity) 283 (M⁺, 17), 268 (7), 240 (10), 215 (24), 200 (100), 177 (6), 150 (8), 121 (9), 91 (6), 77 (8). Exact mass calculated for C₁₈H₂₁NO₂, 283.1572; found, 283.1572.

### 2. (−)-2-[3-3,4-trans-p-Menthadien-(1,8)-yl]-5-(azidomethyl)resorcinol (Compound 3.15)

A stirred mixture of 3.12 (53 mg, 0.14 mmol) and tetramethylquanidinium azide (111 mg, 0.7 mmol) in anhydrous CH₂Cl₂/CH₃NO₂ (3:1 mixture, 3 mL) was refluxed for 12 hours under argon. The reaction mixture was cooled to room temperature and the solvent evaporated under reduced pressure. Purification by flash column chromatography on silica gel gave 3.15 in 71% yield (30 mg). IR (neat) 3420, 2972, 2925, 2097 (s, —N₃), 1627, 1587, 1515, 1443, 1349, 1237, 1215, 1053, 1032, 889, 830 cm ⁻¹; ¹H NMR (500 MHz, CDCl₃) δ: 6.40 (br s, 1H, ArH), 6.30 (br s, 1H, ArH), 6.12 (br s, 1H, OH), 5.56 (s, 1H, 2-H), 4.87 (br s, 1H, OH), 4.64 (s, 1H, 9-H), 4.53 (s, 1H, 9-H), 4.18 (s, 2H, —CH₂N₃), 3.90 (m as br d, J=8.4 Hz, 1H, 3-H), 2.40 (td, J=11.1 Hz, J=3.5 Hz, 1H, 4-H), 2.29-2.20 (m, 1H), 2.15-2.07 (m, 1H), 1.88-1.73 (m and s overlapping, 5H, especially 1.81, s, 7-CH₃), 1.66 (s, 3H, 10-CH₃); mass spectrum m/z (relative intensity) 299 (M⁺, 37), 256 (7), 231 (54), 216 (73), 202 (6), 188 (100), 174 (13), 162 (12), 150 (11), 121 (28), 91 (10), 77 (11). Exact mass calculated for C₁₇H₂₁N₃O₂, 299.1634; found, 299.1631.

### 3. (−)-4-[3-3,4-trans-p-Menthadien-(1,8)-yl]-5-(cyanomethyl)resorcinol (Compound 4.13)

The synthesis was carried out analogous to the preparation of 3.13; yield: 55%; white solid, m p 131-133° C.; IR (neat) 3379, 2931, 2270 (w, —C≡N), 1626, 1603, 1451, 1377, 1337, 1268, 1146, 1050, 1018, 925, 896, 857, 826 cm⁻¹; ¹H NMR (500 MHz, CDCl₃) δ: 6.44 (d, J=2.1 Hz, 1H, ArH), 6.35 (d, J=2.1 Hz, 1H, ArH), 6.20 (s, 1H, OH), 5.54 (s, 1H, 2-H), 4.94 (s, 1H, OH), 4.74 (s, 1H, 9-H), 4.53 (s, 1H, 9-H), 3.69 (d, J=18.1 Hz, 1H, —CH₂CN), 3.50 (d, J=18.1 Hz, 1H, —CH₂CN), 3.40 (m as br d, J=9.6 Hz, 1H, 3-H), 2.42 (td, J=10.8 Hz, J=2.9 Hz, 1H, 4-H), 2.30-2.19 (m, 1H), 2.16-2.08 (m, 1H), 1.90-1.72 (m and s overlapping, 5H, especially 1.81, s, 7-Me), 1.52 (s, 3H, 10-Me); mass spectrum (FAB) m/z (relative intensity) 284 (M⁺1, 22), 258 (15), 154 (87), 136 (100). Exact mass calculated for C₁₈H₂₂NO₂ (M⁻+1) 284.1651; found, 284.1651.

### 4. (−)-4-[3-3,4-trans-p-Menthadien-(1,8)-yl]-5-(azidomethyl)resorcinol (Compound 4.15)

The synthesis was carried out analogous to the preparation of 3.15; yield: 69%; IR (neat) 3409, 2972, 2926, 2096 (s, —N₃), 1621, 1594, 1449, 1243, 1136, 1051, 892, 843, 732 cm⁻¹; ¹H NMR (500 MHz, CDCl₃) δ: 6.37 (d, J=2.5 Hz, 1H, ArH), 6.33 (d, J=2.5 Hz, 1H, ArH), 6.17 (s, 1H, OH), 5.54 (s, 1H, 2-H), 5.15 (s, 1H, OH), 4.67 (s, 1H, 9-H), 4.47 (s, 1H, 9-H), 4.24 (d, J=13.7 Hz, 1H, —CH₂N₃), 4.06 (d, J=13.7 Hz, —CH₂N₃), 3.51 (m as br d, J=9.4 Hz, 1H, 3-H), 2.45 (td, J=10.9 Hz, J=3.2 Hz, 1H, 4-H), 2.29-2.18 (m, 1H), 2.16-2.07 (m, 1H), 1.88-1.72 (m and s overlapping, 5H, especially 1.80, s, 7-Me), 1.52 (s, 3H, 10-Me); mass spectrum m/z (relative intensity) 299 (M⁺, 4), 256 (14), 202 (95), 188 (100), 175 (24), 162 (81), 136 (38), 109 (18), 91 (15), 77 (18). Exact mass calculated for C₁₇H₂₁N₃O₂, 299.1634; found, 299.1637.

Compounds 8.2, 9.2, 11.2 and 12.2 were synthesized by a method depicted in Scheme 9.

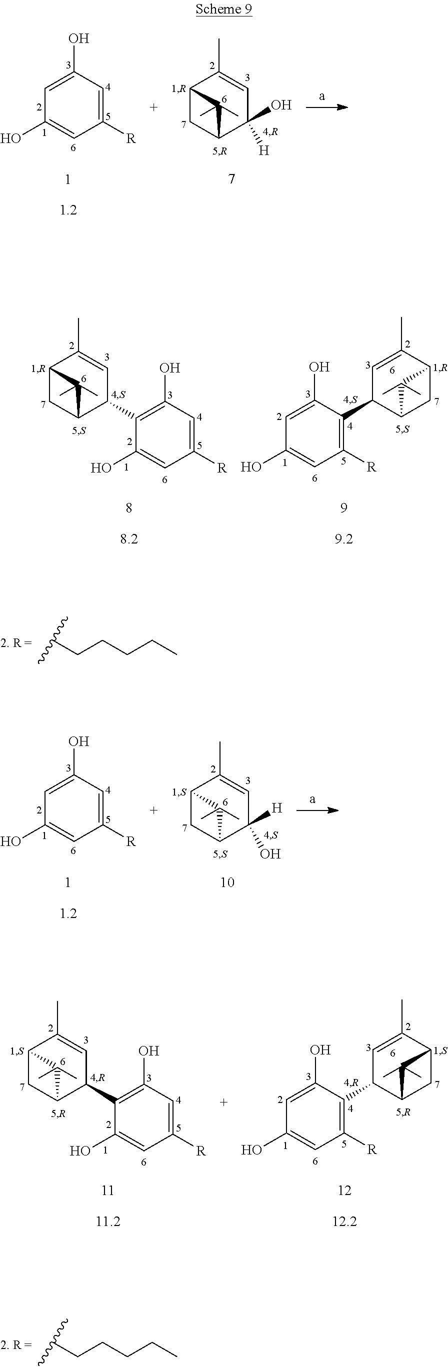

Reagents and conditions: (a) CHCl₃, p-TSA, 0° C. to r t.

### Experimental Procedures:

To a stirred solution of olivetol (368 mg, 2.04 mmol) and p-TSA (39 mg, 0.225 mmol) in anhydrous CHCl₃ (70 ml) at 0° C. under an argon atmosphere, was added a solution of 7 (342 mg, 2.25 mmol) in anhydrous CHCl₃ (12 ml) over a period of 30 min. Following the addition, the reaction temperature was raised to room temperature and stirring was continued for 1.5 hours. The reaction was quenched by the addition of saturated sodium bicarbonate solution, the organic layer was separated and the aqueous phase was extracted with CHCl₃. The combined organic layer was washed with brine, dried (MgSO₄) and the solvent was evaporated under reduced pressure. Purification by flash column chromatography on silica gel (15-45% diethyl ether in hexane) gave compound 8.2 (398 mg, 62% yield) and compound 9.2 (148 mg, 23% yield).

### 1. (+)-2-[(1R, 4S, 5S)-2,6,6-trimethylbicyclo[3.1.1]hept-2-en-4-yl]-5-pentyl-resorcinol (Compound 8.2)

Yellow gum; IR (neat) 3409, 2926, 2859, 1627, 1579, 1516, 1443, 1284, 1229, 1071, 1021, 872, 829 cm⁻¹; ¹H NMR (500 MHz, CDCl₃) δ: 6.20 (br s, 2H, ArH), 5.70 (m, 1H, 3-H), 3.91 (m, 1H, 4-H), 2.44 (dd as t, J=7.7 Hz, 1H, 1′-H), 2.33-2.29 (m, 1H, 7α-H), 2.27-2.24 (m, 1H, 5-H), 2.18 (td, J=5.2 Hz, J=1.3 Hz, 1H, 1-H), 1.85 (dd, J=2.3 Hz, J=1.6 Hz, 3H, C₂—CH₃), 1.60-1.53 (m, 2H, 2′-H), 1.49 (d, J=9.7 Hz, 1H, 7β-H), 1.34-1.28 (m, 7H, 3′-H, 4′-H, especially 1.32, s, CH₃), 0.96 (s, 3H, CH₃), 0.89 (t, J=7.0Hz, 3H, 5′-H).

### 2. (+)-4-[(1R, 4S, 5S)-2,6,6-trimethylbicyclo[3.1.1]hept-2-en-4-yl]-5-pentyl-resorcinol (Compound 9.2)

Yellow gum; ¹H NMR (500 MHz, CDCl₃) δ: 7.28 (s, 1H, OH), 6.24 (d, J=2.7 Hz, 1H, ArH), 6.16 (d, J=2.7 Hz, 1H, ArH), 5.72 (m, 1H, 3-H), 4.55 (s, 1H, OH), 3,71 (m, 1H, 4-H), 2.55-2.49 (m, 1H), 2.39-2.34 (m, 1H), 2.33-2.29 (m, 1H), 2.19 (td, J=5.6 Hz, J=1.2 Hz, 1H), 2.12-2.09 (m, 1H), 1.86 (dd, J=2.3 Hz, J=1.6 Hz, 3H, C₂—CH₃), 1.60-1.51 (m, 3H), 1.38-1.31 (m, 7H, 3′-H, 4′-H, especially 1.32, s, CH₃), 0.59 (s, 3H, CH₃), 0.89 (t, J=7.0 Hz, 3H, 5′-H).

### 3. (−)-2-[(1S, 4R, 5R)-2,6,6-trimethylbicyclo[3.1.1]hept-2-en-4-yl]-5-pentyl-resorcinol (Compound 11.2)

Yield: 61%; yellow gum. The ¹H NMR (500 MHz, CDCl₃) spectrum was identical to that of the enantiomer 8.2.

### 4. (−)-4-[(1S, 4R, 5R)-2,6,6-trimethylbicyclo[3.1.1]hept-2-en-4-yl1-5-pentyl-resorcinol (Compound 12.2)

Yield: 26%; yellow gum. IR (neat) 3402, 2925, 2870, 1620, 1592, 1447, 1328, 1247, 1135, 1008, 843 cm⁻¹. The ¹H NMR (500 MHz, CDCl₃) spectrum was identical to that of the enantiomer 9.2.

Synthetic intermediates 14.1-14-6 as well as compound 15.2 were synthesized by a method depicted in Scheme 10. (3-Methoxyphenyl)methanol (13.1), (3,5-dimethoxyphenyl)methanol (13.2) and 2-(3-methoxyphenyl)ethanol (13.4) were commercially available. 1,3-Dimethoxy-5-(1′-hydroxy[3-(²H₃)]propyl)benzene (13.3) and 3-(3,5-dimethoxyphenyl)-1-propanol (13.5) were synthesized by methods disclosed in Nikas, et al. J. Chem. Soc., Perkin Trans, 1 (2002) 2544-2548 and in Nikas, et al. Synth. Commun. (2002) 1751-1756, the content of which is hereby incorporated by reference. 5-(3,5-Dimethoxyphenyl)pentan-1-ol (13.6) was synthesized by a method depicted in Scheme 11 starting from commercially available 3,5-dimethoxybenzaldehyde (42).

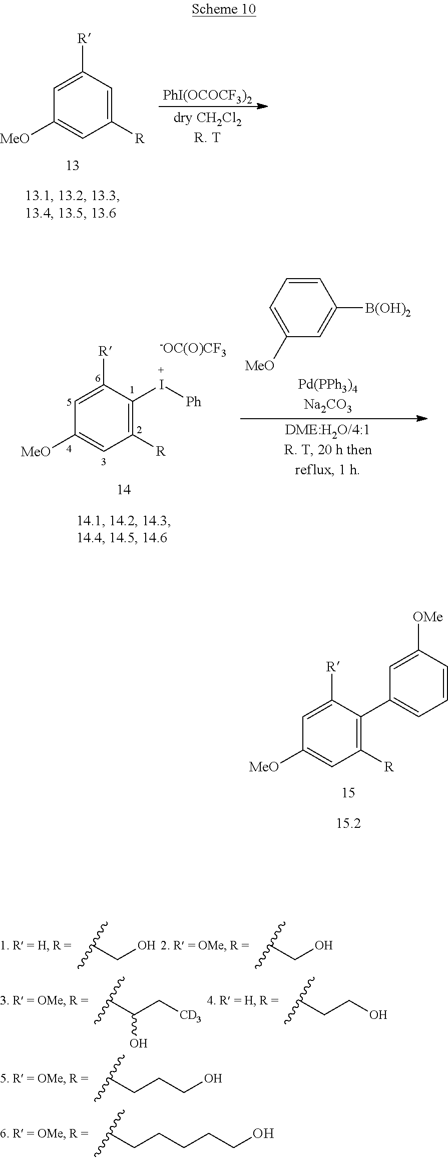

### General Experimental Procedure

To a stirred solution of 13 (1 equiv.) in dry methylene chloride (0.1 M) at room temperature, under an argon atmosphere was added [bis(trifluoroacetoxy)iodo]benzene (1.1 to 1.7 equiv.) Stirring was continued until the TLC analysis indicated total consumption of the starting material (2 hours to 2 days). The solvent was removed in vacuo and the oily residue was treated with CH₂Cl₂ and/or Et₂O to give the product as a white solid which was isolated by filtration (53-78% yields).

### 1. [4-Methoxy-2-(hydroxymethyl)-phenyl](phenyl)iodonium trifluoroacetate (Compound 14.1)

M p=146-147° C.; IR (AgCl) 1657 cm⁻¹; ¹H NMR (500 MHz, CDCl₃) δ: 7.98 (d, J=7.9 Hz, 2H, 2′-H, 6′-H, PhH), 7.73 (t, J=7.9 Hz, 1H, 4′-H, PhH), 7.54 (t, J=7.9 Hz, 2H, 3′-H, 5′-H, PhH), 6.88 (d, J=2.7 Hz, 1H, 3-H, ArH), 6.83 (d, J=9.1 Hz, 1H, 6-H, ArH), 6.72 (dd, J=9.1 Hz, J=2.7 Hz, 1H, 5-H, ArH), 4.98 (s, 2H, —CH₂OH), 3.79 (s, 3H, OMe), 3.45 (br s, 1H, OH); mass spectrum (FAB) m/z (relative intensity) 341 (M⁺-CF₃COO, 82), 309 (44), 155 (100). Exact mass calculated for C₁₄H₁₄IO₂ (M⁺-CF₃COO) 341.0039; found, 341.0036.

### 2. [4,6-Dimethoxy-2-(hydroxymethyl)-phenyl](phenyl)iodonium trifluoroacetate (Compound 14.2)

IR (AgCl) 1672 cm⁻¹; ¹H NMR (500 MHz, CDCl₃) δ: 7.88 (d, J=8.0 Hz, 2H, 2′-H, 6′-H, PhH), 7.50 (t, J=8.0 Hz, 1H, 4′-H, PhH), 7.35 (t, J=8.0 Hz, 2H, 3+-H, 5′-H, PhH), 6.74 (d, J=2.4 Hz, 1H, ArH), 6.41 (d, J=2.4 Hz, 1H, ArH), 4.87 (s, 2H, —CH₂OH), 3.84 (s, 3H, OMe), 3.78 (s, 3H, OMe), 3.30 (br s, 1H, OH).

### 3. [4,6-Dimethoxy-2-(1-hydroxy-3,3,3-(²H₃)-propyl)-phenyl](phenyl)iodonium trifluoroacetate (Compound 14.3)

IR (AgCl) 2224, 1666 cm⁻¹; ¹H NMR (500 MHz, CDCl₃) δ: 7.83 (d, J=7.8 Hz, 2H, 2′-H, 6′-H, PhH), 7.51 (t, J=7.8 Hz, 1H, 4′-H, PhH), 7.37 (t, J=7.8 Hz, 2H, 3′-H, 5′-H, PhH), 6.73 (d, J=2.6 Hz, 1H, ArH), 6.41 (d, J=2.6 Hz, 1H, ArH), 4.97 (dd, J=7.8 Hz, J=5.6 Hz, 1H, —CH(OH)—), 3.85 (s, 3H, OMe), 3.77 (s, 3H, OMe), 2.78 (br s, 1H, OH), 1.88 (dd, J=13.7 Hz, J=7.8 Hz, 1H, —CH₂CD₃), 1.70 (dd, J=13.7 Hz, J=5.6 Hz, 1H, —CH₂CD₃).

### 4. [4-Methoxy-2-(2-hydroxy-ethyl)-phenyl](phenyl)iodonium trifluoroacetate (Compound 14.4)

IR (AgCl) 1671 cm⁻¹; ¹H NMR (500 MHz, CDCl₃) δ: 7.90 (d, J=7.8 Hz, 2H, 2′-H, 6′-H, PhH), 7.58 (t, J=7.8 Hz, 1H, 4′-H, PhH), 7.48 (d, J=9.0 Hz, 1H, 6-H, ArH), 7.43 (t, J=7.8 Hz, 2H, 3′-H, 5′-H, PhH), 6.93 (d, J=2.8 Hz, 1H, 3-H, ArH), 6.73 (dd, J=9.0 Hz, J=2.8 Hz, 1H, 5-H, ArH), 3.89 (t, J=5.3 Hz, 2H, —CH₂CH₂OH), 3.81 (s, 3H, OMe), 3.25 (br s, 1H, OH), 3.08 (t, J=5.3 Hz, 2H, —CH₂CH₂OH).

### 5. [4,6-Dimethoxy-2-(3-hydroxy-propyl)-phenyl](phenyl)iodonium trifluoroacetate (Compound 14.5)

IR (AgCl) 1647, 1579 cm⁻¹; ¹H NMR (500 MHz, CDCl₃) δ: 7.89 (d, J=7.6 Hz, 2H, 2′-H, 6′-H, PhH), 7.40 (t, J=7.6 Hz, 1H, 4′-H, PhH), 7.29 (t, J=7.6 Hz, 2H, 3′-H, 5′-H, PhH), 6.53 (d, J=2.5 Hz, 1H, ArH), 6.34 (d, J=2.5 Hz, 1H, ArH), 3.84 (s, 3H, OMe), 3.82 (s, 3H, OMe), 3.64 (t, J=5.7 Hz, 2H, —CH₂OH), 3.14 (t, J=7.3 Hz, 2H, —CH₂CH₂CH₂OH), 1.99 (qt, J=6.8 Hz, 2H, —CH₂CH₂CH₂OH).

### 6. [4,6-Dimethoxy-2-(5-hydroxy-pentyl)-phenyl](phenyl)iodonium trifluoroacetate (Compound 14.6)

M p=119-121° C.; ¹H NMR (500 MHz, CDCl₃) δ: 7.82 (d, J=8.1 Hz, 2H, 2′-H, 6′-H, PhH), 7.49 (t, J=8.1 Hz, 1H, 4′-H, PhH), 7.36 (t, J=8.1 Hz, 2H, 3′-H, 5′-H, PhH), 6.54 (d, J=2.5 Hz, 1H, ArH), 6.39 (d, J=2.5 Hz, 1H, ArH), 3.87 (s, 3H, OMe), 3.86 (s, 3H, OMe), 3.64 (t, J=6.2 Hz, 2H, —CH₂OH), 2.90 (m as t, J=7.7 Hz, 2H, Ar—CH₂—), 2.12 (br s, 1H, OH), 1.66 (qt, J=7.7 Hz, 2H), 1.60 (qt, J=7.5 Hz, 2H), 1.48 (qt, J=7.1 Hz, 2H); mass spectrum (FAB) m/z (relative intensity) 427 (M⁺-CF₃COO, 100), 350 (8), 225 (13). Exact mass calculated for C₁₉H₂₄IO₃ (M⁺-CF₃COO) 427.0770; found, 427.0772.

### 7. 4,6,3′-Trimethoxy-[1,1′-biphenyl]-2-methanol (Compound 15.2)

A degassed mixture of iodonium salt 14.2 (484 mg, 1 mmol), 3-methoxyphenyl boronic acid (182 mg, 1.2 mmol), Pd(PPh₃)₄ (115 mg, 0.1 mmol) and Na₂CO₃ (212 mg, 2 mmol) in DME (4 ml)/water (1 ml) was flushed with argon, and stirred at room temperature for 20 hours and under reflux for 1 hour. The reaction mixture was cooled to room temperature, diluted with saturated NH₄Cl solution and diethyl ether, and filtered through a short pad of Celite. The organic layer was separated and the aqueous phase was acidified with 5% HCl and extracted with diethyl ether. The combined organic layer was washed with brine, dried over MgSO₄ and concentrated in vacuo. Purification by flash column chromatography on silica gel (40% ethyl acetate in hexane) gave 90 mg (33% yield) of the title compound.

¹H NMR (500 MHz, CDCl₃) δ: 7.32 (dd as t, J=8.1 Hz, 1H, 5’-H), 6.89 (ddd, J=8.1 Hz, J=2.6 Hz, J=0.8 Hz, 1H, 6′-H), 6.81 (ddd, J=8.1 Hz, J=1.5 Hz, J=0.8 Hz, 1H, 4′-H), 6.79 (dd, J=2.6 Hz, J=1.5 Hz, 1H, 2′-H), 6.73 (d, J=2.4 Hz, 1H, (MeO)₂PhH—), 6.50 (d, J=2.4 Hz, 1H, (MeO)₂PhH—), 4.43 (s, 2H, —CH₂OH), 3.87 (s, 3H, OMe), 3.81 (s, 3H, OMe), 3.71 (s, 3H, OMe).

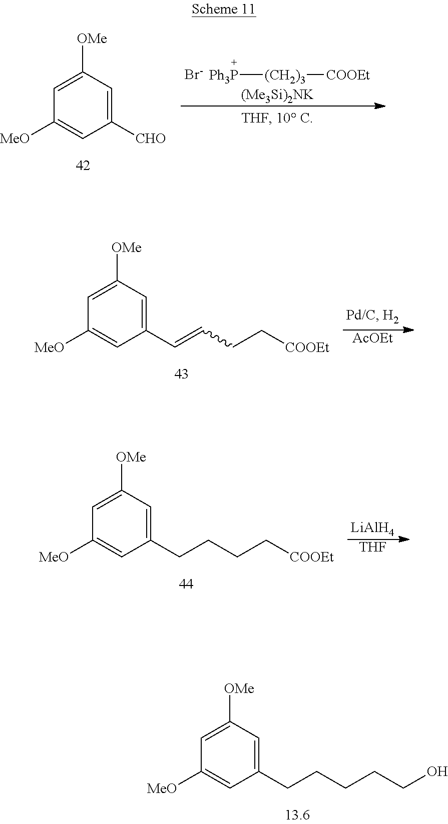

### Experimental Procedures:

### 1. Ethyl 5-(3,5-dimethoxyphenyl)pent-4-enoate (Compound 43)

To a suspension of [3-(ethoxycarbonyl)propyl]triphenylphosphonium bromide (24.7 g, 54 mmol) in anhydrous THF (270 ml) at 0° C. under an argon atmosphere, was added potassium bis(trimethylsilyl)amide (10.4 g, 52.2 mmol). The mixture was warmed to 10° C. and stirred for 40 min to ensure complete formation of the orange glide. A solution of 3,5-dimethoxybenzaldehyde (3 g, 18 mmol) in anhydrous THF (20 ml) was added dropwise at the same temperature. The reaction was stirred for 1.5 h and upon completion was quenched by the addition of saturated aqueous NH₄Cl solution. The organic layer was separated and the aqueous phase was extracted with diethyl ether. The combined organic layer was washed with brine and dried over MgSO₄, and the solvent was evaporated under reduced pressure. Purification by flash column chromatography on silica gel (25% diethyl ether in hexane) gave compound 43 (4.58 g, colorless viscous oil, 96% yield) as a mixture of cis and trans isomers in 97:3 ratio, respectively.

¹H NMR (500 MHz, CDCl₃) δ: 6.43 (d, J=2.1 Hz, 2H, 2-H, 6-H of the cis isomer), 6.40 (d, J=11.7 Hz, 1H, —CH═CH—CH₂—CH₂— of the cis isomer), 6.36 (t, J=2.1 Hz, 1H, 4-H of the cis isomer), 6.20 (dt, J=15.7 Hz, J=6.5 Hz, 1H, —CH═CH—CH₂—CH₂— of the trans isomer), 5.62 (dt, J=11.7 Hz, J=7.0 Hz, 1H, —CH═CH—CH₂—CH₂— of the cis isomer), 4.13 (q, J=7.2 Hz, 2H, —OCH₂CH₃ of the cis isomer), 3.79 (s, 6H, OMe of the cis isomer), 2.66 (td as q, J=7.1 Hz, 2H, —CH═CH—CH₂—CH₂— of the cis isomer), 2.43 (t, J=7.3 Hz, 2H, —CH═CH—CH₂—CH₂— of the cis isomer), 1.24 (t, J=7.2 Hz, 3H, —OCH₂CH₃ of the cis isomer).

### 2. Ethyl 5-(3,5-dimethoxyphenyl)pentanoate (Compound 44)

To a solution of 43 (2.7 g, 10.22 mmol) in ethyl acetate (96 ml) was added 10% Pd/C (0.46 g) and the suspension was stirred vigorously under a hydrogen atmosphere for 3 h at room temperature. The catalyst was removed by filtration through a short pad of Celite and the filtrate was evaporated under reduced pressure to give pure ester 44 as oil in 97% yield (2.65 g).

¹H NMR (200 MHz, CDCl₃) δ: 6.34 (d, J=2.1 Hz, 2H, 2-H, 6-H), 6.30 (t, J=2.1 Hz, 1H, 4-H), 4.12 (q, J=7.1 Hz, 2H, —OCH₂CH₃), 3.78 (s, 6H, OMe), 2.57 (t, J=7.1 Hz, 2H), 2.32 (t, J=7.0 Hz, 2H), 1.77-1.54 (m, 4H), 1.25 (t, J=7.1 Hz, 3H, —OCH₂CH₃).

### 3. 5-(3,5-Dimethoxyphenyl)pentan-1-ol (Compound 13.6)

To a stirred suspension of LAH (0.844 g, 22.2 mmol) in anhydrous THF (14 ml) at 0° C. under argon atmosphere, was added a solution of 44 (1.97, 7.4 mmol) in anhydrous THF (8 ml) over a period of 10 min. The reaction mixture was stirred vigorously for 45 min at the same temperature and then quenched by adding NaF (1 g, 24 mmol) followed by dropwise addition of 10% aqueous NaOH and water. The mixture was then warmed to room temperature, diluted with ethyl acetate and stirred for an additional 20 min. The suspension was filtered through a short pad of Celite and the organic layer was separated. The aqueous phase was extracted with ethyl acetate and the combined organic layer was washed with brine, dried over MgSO₄, and the solvent was evaporated under reduced pressure. Purification by flash column chromatography on silica gel (50% ethyl acetate in hexane) gave compound 13.6 (1.56 g, 94% yield) as colorless oil.

¹H NMR (200 MHz, CDCl₃) δ: 6.34 (d, J=2.1 Hz, 2H, 2-H, 6-H), 6.30 (t, J=2.1 Hz, 1H, 4-H), 3.78 (s, 6H, OMe), 3.64 (t, J=6.4 Hz, 2H, —CH₂OH), 2.57 (t, J=7.8 Hz, 2H, Ar—CH₂—), 1.75-1.51 (m, 4H), 1.49-1.30 (m, 2H).

### Biological Testing Results

TABLE 4
Angiogenesis data of some synthesized compounds of the general formulas I, II and III.

| | Compound | Cord formation assay | | Sprouting assay |
|---|---|---|---|---|
| | number | % Control | | Angiogeneic score |
| Structure | (concentration) | Length | Junction # | (arbitrary unit) |
| 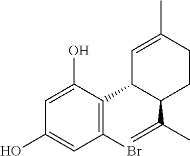 | 4.10 (1 × 10⁻⁸ M) (1 × 10⁻⁷ M) (1 × 10⁻⁶ M) | 14 21 33 | 6 9 14 | 0.25 0.5 1.0 |
| 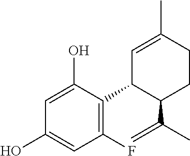 | 4.8 (1 × 10⁻⁸ M) (1 × 10⁻⁷ M) (1 × 10⁻⁶ M) | 12 27 37 | 9 14 20 | 0.5 1.0 1.5 |
| 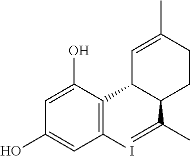 | 4.11 (1 × 10⁻⁸ M) (1 × 10⁻⁷ M) (1 × 10⁻⁶ M) | 10 19 39 | 7 11 19 | 0.25 1.0 1.0 |
| 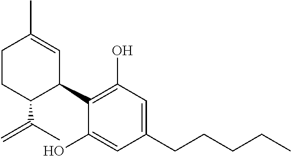 | 3.2 (1 × 10⁻⁸ M) (1 × 10⁻⁷ M) (1 × 10⁻⁶ M) | 10 15 20 | 5 7 15 | 0.0 0.25 0.5 |
| 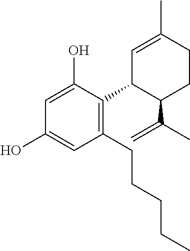 | 4.2 (1 × 10⁻⁸ M) (1 × 10⁻⁷ M) (1 × 10⁻⁶ M) | 11 15 29 | 6 9 15 | 0.0 0.5 1.0 |
| 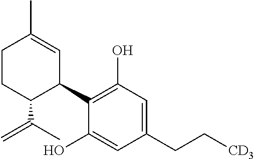 | 3.5 (1 × 10⁻⁸ M) (1 × 10⁻⁷ M) (1 × 10⁻⁶ M) | 7 11 23 | NC 6 11 | 0.0 0.25 0.5 |
| 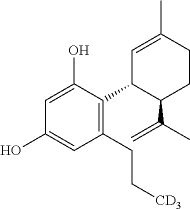 | 4.5 (1 × 10⁻⁸ M) (1 × 10⁻⁷ M) (1 × 10⁻⁶ M) | 7 14 32 | 4 9 13 | 0.25 0.5 1.0 |
| 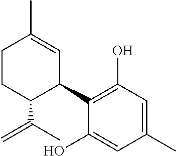 | 3.1 (1 × 10⁻⁸ M) (1 × 10⁻⁷ M) (1 × 10⁻⁶ M) | NC 14 26 | NC 6 15 | 0.25 0.5 1.5 |
| 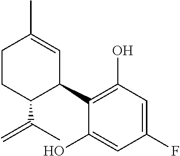 | 3.8 (1 × 10⁻⁸ M) (1 × 10⁻⁷ M) (1 × 10⁻⁶ M) | 9 17 31 | 3 11 13 | 0.25 0.5 >1 |
| 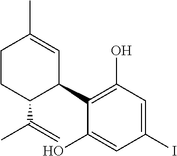 | 3.11 (1 × 10⁻⁸ M) (1 × 10⁻⁷ M) (1 × 10⁻⁶ M) | 8 18 28 | 5 7 8 | 0.25 0.25 0.5 |
| 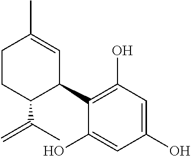 | 3.7 (1 × 10⁻⁸ M) (1 × 10⁻⁷ M) (1 × 10⁻⁶ M) | 8 12 18 | 3 6 10 | 0.0 0.25 0.25 |
| 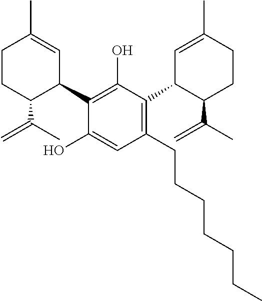 | 5.3 (1 × 10⁻⁸ M) (1 × 10⁻⁷ M) (1 × 10⁻⁶ M) | 20 32 43 | 8 12 18 | 0.5 1.0 1.5 |
| 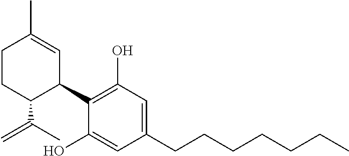 | 3.3 (1 × 10⁻⁸ M) (1 × 10⁻⁷ M) (1 × 10⁻⁶ M) | 10 20 28 | 7 13 19 | 0.25 0.5 0.5-1.0 |
| 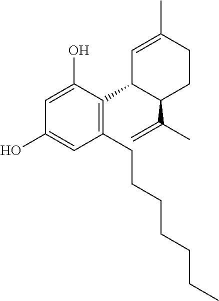 | 4.3 (1 × 10⁻⁸ M) (1 × 10⁻⁷ M) (1 × 10⁻⁶ M) | 12 21 31 | 7 14 20 | 0.0 0.5 0.5-1.0 |
| 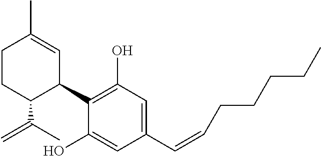 | 3.6 (1 × 10⁻⁸ M) (1 × 10⁻⁷ M) (1 × 10⁻⁶ M) | 7 13 26 | NC 7 11 | 0.0 0.25 0.5 |
| 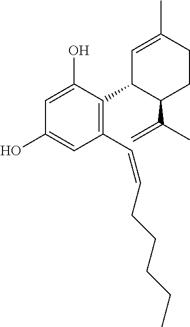 | 4.6 (1 × 10⁻⁸ M) (1 × 10⁻⁷ M) (1 × 10⁻⁶ M) | 7 17 32 | 4 7 16 | 0.0 0.5 0.5-1.0 |
| 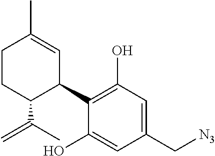 | 3.15 (1 × 10⁻⁸ M) (1 × 10⁻⁷ M) (1 × 10⁻⁶ M) | 15 27 47 | 7 10 14 | 0.25 0.5 2.0 |
| 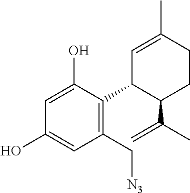 | 4.15 (1 × 10⁻⁸ M) (1 × 10⁻⁷ M) (1 × 10⁻⁶ M) | 9 14 29 | 3 8 17 | 0.25 0.5-1.0 >1 |
| 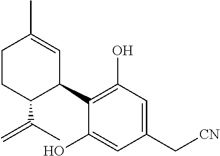 | 3.13 (1 × 10⁻⁸ M) (1 × 10⁻⁷ M) (1 × 10⁻⁶ M) | 12 18 21 | 5 5 7 | 0.25 0.5 0.5 |
| 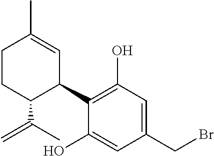 | 3.12 (1 × 10⁻⁸ M) (1 × 10⁻⁷ M) (1 × 10⁻⁶ M) | 6 17 31 | NC 5 12 | NC 0.5 0.5-1.0 |

TABLE 5
Affinities (Ki) of some synthesized compounds for CB1 and CB2 cannabinoid receptors.

| | | CB1 | CB2 |
|---|---|---|---|
| Compound | | Receptor Ki | Receptor Ki |
| number | Structure | (nM) | (nM) |
| 3.1 | 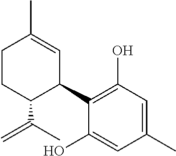 | 16,690 | 47,740 |
| 3.2 | 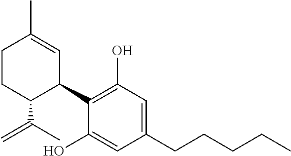 | 1,496 | 286 |
| 3.3 | 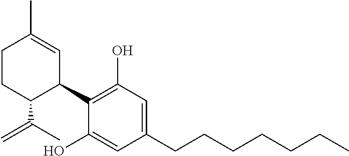 | 651 | 1416 |
| 3.4 | 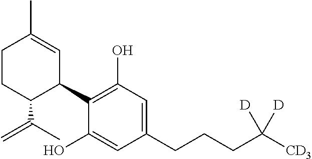 | NT | NT |
| 3.5 | 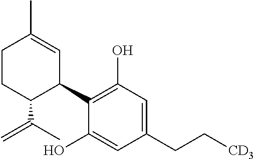 | 8,390 | 2,581 |
| 3.6 | 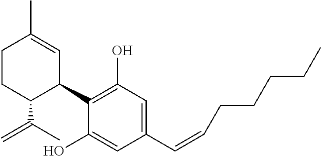 | NT | NT |
| 3.7 | 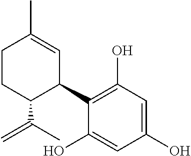 | 21,545 | 41,712 |
| 3.8 | 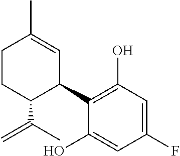 | 14,590 | 43,380 |
| 3.11 | 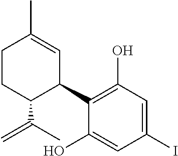 | 5,131 | 2,191 |
| 3.12 | 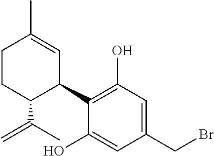 | 2,199 | 10,848 |
| 3.13 | 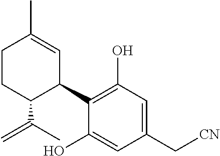 | 1,440 | 4,202 |
| 3.14 | 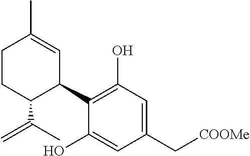 | 2,366 | 620 |
| 3.15 | 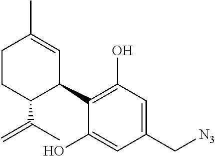 | 3,559 | 2,035 |
| 3.16 | 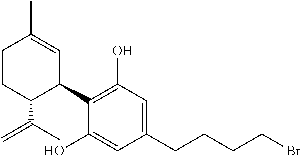 | 192 | 59 |
| 8.2 | 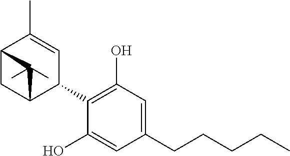 | NT | NT |
| 11.2 | 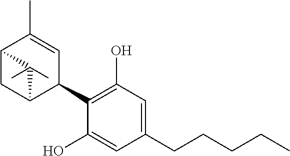 | 837 | 565 |
| 4.1 | 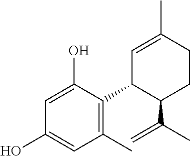 | 10,040 | 31,120 |
| 4.2 | 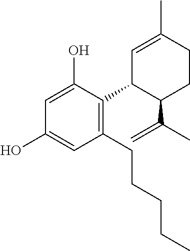 | 4,778 | 9,643 |
| 4.3 | 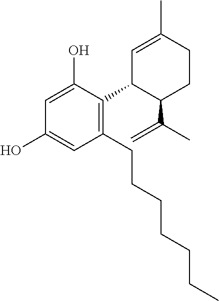 | 250 | 398 |
| 4.4 | 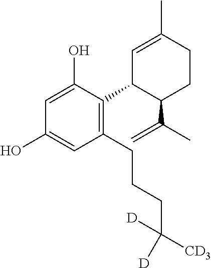 | NT | NT |
| 4.5 | 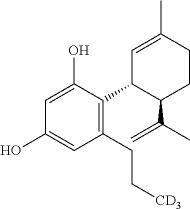 | >20,000 | 19,000 |
| 4.6 | 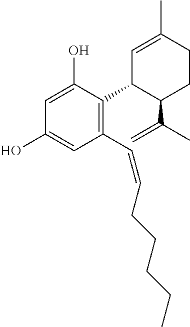 | NT | NT |
| 4.8 | 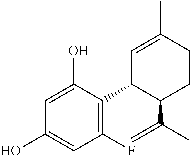 | 4,154 | 7,210 |
| 4.9 | 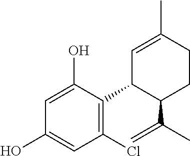 | 3,177 | 4,974 |
| 4.10 | 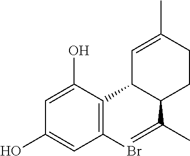 | 7,460 | 13,260 |
| 4.11 | 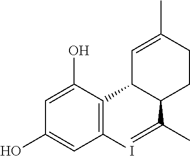 | 13,110 | 10,500 |
| 4.12 | 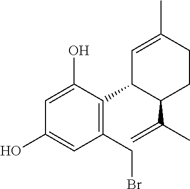 | NT | NT |
| 4.13 | 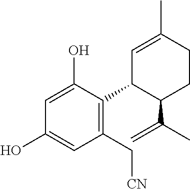 | NT | NT |
| 4.14 | 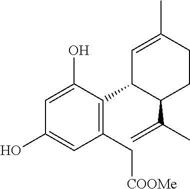 | NT | NT |
| 4.15 | 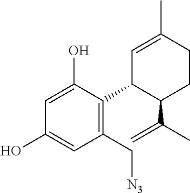 | NT | NT |
| 4.16 | 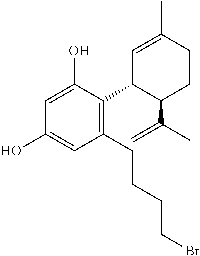 | 12,520 | 5,732 |
| 9.2 | 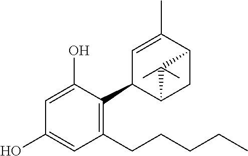 | 9,250 | 4,939 |
| 12.2 | 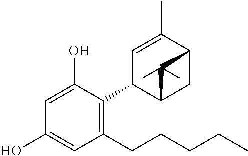 | 10,080 | 19,010 |
| 14.2 | 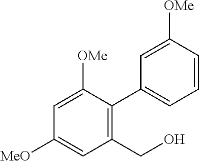 | NT | NT |
| 5.3 | 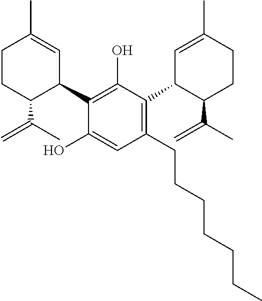 | >10,000 | >10,000 |
| 5.14 | 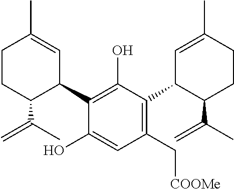 | NT | NT |
| 6.10 | 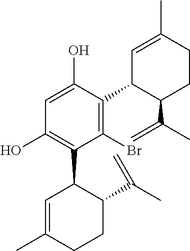 | NT | NT |
| 6.11 | 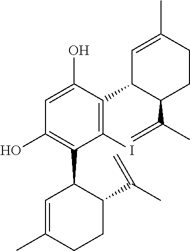 | NT | NT |

### Equivalents

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of formula I below, and any pharmaceutically acceptable salt thereof including all stereoisomers. 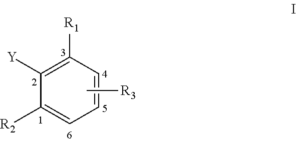
wherein:
R₁ and R₂ are each independently selected from —H, —F, —Cl, —Br, —I, —OH, —SH, —NH₂, —NO₂, —CN, —CF₃, —OC(O)CH₃, —C(O)CH₃, —C(O)CF₃, —C(O)CH═CHCOOH, —O-alkyl, —S-alkyl, —NH-alkyl, —NH(alkyl)₂, —O—P(O)(OR)₂ or —O—P(O)(OH)(OR) (where R is selected from H or alkyl), —P(O)(OR)₂ or —P(O)(OH)(OR) (where R is selected from H or alkyl), —O-alkyl-COOR (where R is selected from H or alkyl), —O-alkyl-NR₄R₅, —O-alkyl-CONR₄R₅, —OC(O)—CH(NH₂)—R₆ (where R₆ is selected from H, CH(OH)CH₃ or alkyl-X₁ and X₁ is selected from: H, —NH—C(═NH)NH₂, C(O)NH₂, COOH, SH, SCH₃, OH, NH₂, a substituted or unsubstituted aromatic ring, a substituted or unsubstituted heteroaromatic ring, a substituted or unsubstituted heterocyclic ring).
R₄ and R₅ are each independently selected from H, alkyl, hydroxyalkyl or R₄ and R₅ together comprise part of a 3 to 7 membered saturated heterocyclic ring containing up to one additional heteroatom selected from N, O and S.
R₃ is selected from —H, —F, —Cl, —Br, —I, —OH, —SH, —NH₂, —CN, —N₃, —NCS, —NCO, —SO₂Cl, —SO₂F, —CONH₂, —SO₂NH₂, —COOH, —NO₂, —CHO, —CF₃, —SO₃H, —O—P(O)(OH)₂, —Sn(alkyl)₃, —Si(alkyl)₃, —C≡CH, —CH₂—C≡CH, —CH═CH₂, -fluoroalkyl, -alkyl-R₆, —Z-alkyl-R₆, -alkyl-Z-alkyl-R₆, and R₃ can occupy any position selected from 4, 5 and 6 in formula I.
R₆ is selected from —H, —F, —Cl, —Br, —I, —OH, —SH, —NH₂, —CN, —N₃, —NCS, —NCO, —SO₂Cl, —SO₂F, —CONH₂, —SO₂NH₂, —COOH, —NO₂, —CHO, —CF₃, —SO₃H, —O—P(O)(OH)₂, —Sn(alkyl)₃, —Si(alkyl)₃, —C≡CH, —CH═CH₂.
Z is selected from —C≡C—, —CH═CH—, —O—, —S—, —NH—, —C(O)—, —C(O)O—, —OC(O)—, —C(O)NH—, —NHC(O)—, —S(O)—, —SO₂—, —SO₂NH—, —NHSO₂—, —SO₂O— and —OSO₂—.
Y is selected from the following structures: 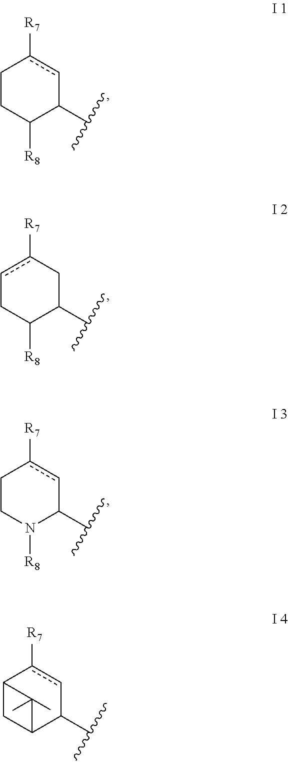
wherein:
The dashed lines independently represent either a single or a double bond.
R₇ is selected from —H, -alkyl, -alkyl-R₉, -alkyl-O-alkyl, -alkyl-O-alkyl-R₉, —C(O)O-alkyl.
R₈ is selected from —H, -alkyl, -alkyl-R₉.
R₉ is selected from —F, —Cl, —Br, —I, —OH, —SH, —NH₂, —NH-alkyl, —N(alkyl)₂, —CN, —N₃, —NCS, —CONH₂, —OC(O)CH₃, —C(O)OCH₃, —SO₂NH₂, —COOH, —NO₂, —CHO, —CF₃, —Sn(alkyl)₃, —Si(alkyl)₃, —C≡CH, —CH═CH₂.
With the following provisos:
If Y is I 1 where the dashed line represents a double bond, R₇ is -Me and R₈ is isopropenyl, R₁ is —O—C₁₋₅alkyl or —O—C₁₋₅alkyl-NR₄R₅, and R₂ is —O—C₁₋₅alkyl or —O—Cᵢ₋₅alkyl-NR₄R₅; then R₃ can not be —H, —F, —Cl, —Br, —I, —C₁₋₃alkyl and —C₁₋₃alkyl-R₆.
If Y is I 1 where the dashed line represents a double bond, R₇ is -Me and R₈ is isopropenyl, R₁ is —OH, and R₂ is —OH; then R₃ can not be —H, and —(CH₂)nCH₃ where n=0-9.

2. The compound of claim 1, wherein R₁ and R₂ are each independently selected from —OH and —SH.

3. The compound of claim 1, wherein R₁ and R₂ are each independently selected from —O-alkyl and —S-alkyl.

4. The compound of claim 1, wherein R₃ is selected from —H, —F, —Cl, —Br, —I, —OH, —SH, and —NH₂.

5. (canceled)

6. The compound of claim 1, wherein R₃ is selected from —OH, —SH, —NH₂, —CN, —N₃, —NCS, and —NCO.

7. (canceled)

8. The compound of claim 1, wherein R₃ is selected from —SO₂Cl, —SO₂F, —CONH₂, —SO₂NH₂, —COOH, —COOH, —NO₂, —CHO, —CF₃, and SO₃H.

9. (canceled)

10. The compound of claim 1, wherein R₃ is selected from —C≡CH, —CH₂—C≡CH, —CH═CH₂, and -alkyl-R₆.

11-12. (canceled)

13. The compound of claim 1, wherein R₆ is selected from —H, —F, —Cl, —Br, and —I.

14. The compound of claim 1, wherein R₆ is selected from —OH, —SH, —NH₂, —CN, —N₃, —NCS, and —NCO.

15. The compound of claim 1, wherein R₆ is selected from —SO₂Cl, —SO₂F, —CONH₂, —SO₂NH₂, —COOH, —NO₂, —CHO, —CF₃, and —SO₃H.

16. The compound of claim 1, wherein R₇ is selected from -alkyl and -alkyl-R₉.

17. (canceled)

18. The compound of claim 16, wherein R₉ is selected from —F, —Cl, —Br, and —I.

19. The compound of claim 16, wherein R₉ is selected from —OH, —SH, —NH₂, and —NH-alkyl.

20. The compound of claim 16, wherein R₉ is selected from —N(alkyl)₂, —CN, —N₃, —NCS, and —CONH₂.

21. The compound of claim 16, wherein R₉ is selected from —OC(O)CH₃, —C(O)OCH₃, —SO₂NH₂, —COOH, —NO₂, —CHO, and —CF₃.

22. The compound of claim 1, wherein R₈ is selected from —alkyl.

23. A pharmaceutical composition comprising a physiologically acceptable excipient and a therapeutically effective amount of a compound of claim 1.

24-26. (canceled)

27. A method of treating a condition selected from high blood pressure disease or hypertension; peripheral vascular disease; coronary artery disease; abnormal heart rate; pulmonary hypertension; ocular hypertension or glaucoma; diseases where hypotension is the result of the action of endogenous cannabinoids and drug-induced vasoconstriction is desirable, for example in hypotensive states, such as shock; vasodilatory shock (caused by vascular dilation, as seen for example in cerebral trauma, drug intoxication, heat exposure or septic shock accompanying a gram negative bacterial infection); cardiogenic shock (for example from arrhythmia or heart failure); to achieve selective hemostasis to stop bleeding induced by trauma or surgery; to treat angiogenesis-dependent events involved both in physiological and pathological conditions such as wound healing, placental development, stroke related blockage of blood capillaries, rheumatoid arthritis, diabetic retinopathy and tumor growth; in an individual or animal having the condition, comprising administering the pharmaceutical composition of claim 23 to the individual or animal.

28. (canceled)

29. A method of modulating the non-CB1/non-CB2 cannabinoid receptors in an individual or animal comprising administering to the individual or animal a therapeutically effective amount of a compound of claim 1.

30-86. (canceled)
